(19) United States
(12) Patent Application Publication
(10) Pub. No.: US 2011/0195083 A1
(43) Pub. Date: Aug. 11, 2011

(54) CYCLIC V3 PEPTIDES FOR ANTI HIV-1 VACCINE

(75) Inventors: Jacob Anglister, Rehovot (IL); Osnat Rosen, Rehovot (IL); Adi Moseri, Rehovot (IL)

(73) Assignee: YEDA RESEARCH AND DEVELOPMENT CO. LTD., REHOVOT (IL)

(21) Appl. No.: 13/122,972

(22) PCT Filed: Sep. 30, 2009
(86) PCT No.: PCT/IL09/00938

§ 371 Date: Apr. 7, 2011

(51) Int. Cl. A61K 39/00; C07K 14/00; C07H 21/00; C12N 15/63; A61P 37/04; A61P 31/18

(52) U.S. Cl. 424/185.1; 530/317; 536/23.1; 435/320.1

(57) ABSTRACT

Isolated cyclic polypeptides useful as vaccinations for the treatment/prevention of HIV are disclosed. An exemplary peptide comprises at least 18 consecutive amino acids of a V3 domain of gp120, starting at position 303 and ending at position 322, the positioning being according to a numbering of the V3 domain of gp120 in a HXB2 strain, wherein amino acids at position 303 and 322 are bonded. Vaccines comprising same and methods of treating AIDS using same are also disclosed.

### FIELD AND BACKGROUND OF THE INVENTION

The present invention, in some embodiments thereof, relates to cyclic peptides and uses thereof for the treatment and/or prevention of AIDS.

In the last two decades since researchers identified HIV as the cause of AIDS, more money has been spent on the search for a vaccine against the virus than on any vaccine effort in history. The U.S. National Institutes of Health alone invests nearly $500 million each year, and more than 50 different preparations have entered clinical trials. Yet an effective AIDS vaccine, which potentially could thwart millions of new HIV infections each year, remains a distant dream.

It is well known that a significant fraction of strain-specific virus-neutralizing antibodies in the serum of HIV-1-infected individuals recognize the third hypervariable loop (V3) domain of the surface subunit of the envelope glycoprotein (gp120) of HIV-1. This region is involved in gp120 binding to the chemokine receptors CCR5 and CXCR4, which serve as co-receptors in HIV-1 infection. The sequence of V3 determines whether the virus binds to CCR5 and infects predominantly macrophages (“R5 virus”) or to CXCR4 and infects mostly T-cells (“X4 virus”). Antibodies targeted against V3 prevent the binding of gp120 to the chemokine receptors, thus blocking events leading to viral fusion (2, 3).

V3 peptides have been investigated as a potential anti-HIV-1 vaccine and a few studies using HIV-1 and SHIV V3 peptides have demonstrated the induction of HIV-1 neutralizing antibodies that neutralize homologous primary isolates (4-8).

A number of additional scientific reports attempted neutralization of HIV by generation of anti-HIV-1 vaccine using HIV-1 V3 peptides, some are summarized in the following section.

Haynes et al (9) teaches an immunogen of 22 residues based on the V3 segment (in the form of a C4-V3 peptide, where C4 stands for the fourth constant region of gp120), that resembles the consensus sequence of Glade-B R5 viruses. This immunogen was found to induce antibodies that neutralize 31% of the subtype-B HIV-1 isolates that were evaluated

In another approach, tandem copies of V3 loops derived from various strains of HIV-1 were fused together at the gene level to produce a multi-strain V3 loop antigen [Aguilar A., et al., Biomol. Eng. 2001; 18:117-124].

WO2004075850 teaches linear peptide immunogens capable of eliciting a broad neutralizing response.

Cyclic peptides have also been used for immunization in an attempt to mimic the probable V3 conformation. Tolman, R. L. et al., [Int. J. Pept. Protein Res. 1993; 41:455-466] and Conley, A. et al., [Vaccine. 1994; 12:445-451] teach cyclic peptides whose sequences are derived from X4 viruses.

Richalet-Secordel, P. et al., [FEMS Immunol. Med. Microbiol. 1994; 9:77-87; J. Immunol. Methods. 1994; 176:221-234] teach a 35 amino acid cyclic peptide whose sequences are derived from JR-FL viruses. The peptide is constrained at the original V3 base.

WO2004069863 teaches constrained HIV V3 loop peptides as immunogens and receptor antagonists.

447-52D is a monoclonal antibody that recognizes the conserved tip of the V3 loop in a β-turn conformation. This antibody has previously been shown to neutralize diverse strains of the virus. In an attempt to generate an immunogen competent to generate 447-52D-like antibodies, Varadarajan and co-workers (2l) inserted the known epitope of 447-52D at different surface loop locations in the small, stable protein Escherichia coli Trx (thioredoxin). The epitope was constrained using a disulfide bond. The constrained V3-thioredoxin molecule bound 447-52D with affinity comparable to that of gp120.

### SUMMARY OF THE INVENTION

According to an aspect of some embodiments of the present invention there is provided an isolated cyclic polypeptide comprising a single internal constraint, comprising at least eighteen consecutive amino acids of a V3 domain of gp120, starting at position 303 and ending at position 322, said positioning being according to a numbering of said V3 domain of gp120 in a HXB2 strain, wherein amino acids at position 303 and 322 are bonded.

According to an aspect of some embodiments of the present invention there is provided an isolated cyclic polypeptide comprising at least nineteen consecutive amino acids of a V3 domain of gp120, starting at position 303 and ending at position 323, said positioning being according to a numbering of said V3 domain of gp120 in a HXB2 strain, wherein amino acids at position 303 and 323 are bonded.

According to an aspect of some embodiments of the present invention there is provided an isolated cyclic polypeptide comprising at least 23 consecutive amino acids of a V3 domain of gp120, starting at position 298 and ending at position 322, said positioning being according to a numbering of said V3 domain of gp120 in a HXB2 strain, wherein amino acids at position 303 and 322 are bonded.

According to an aspect of some embodiments of the present invention there is provided an isolated cyclic polypeptide comprising at least 22 consecutive amino acids of a V3 domain of gp120, starting at position 301 and ending at position 324/325, said positioning being according to a numbering of said V3 domain of gp120 in a HXB2 strain, wherein amino acids at position 301 and 324/325 are bonded.

According to an aspect of some embodiments of the present invention there is provided an isolated cyclic polypeptide comprising an amino acid consensus sequence X₁X₂X₃X₄X₅X₆X₇X₈X₉X₁₀X₁₁X₁₂X₁₃X₁₄X₁₅X₁₆X₁₇X₁₈X₁₉, where X₁ and X₁₉ are bonded, X₈ is glycine, X₉ is proline and X₁₀ is glycine.

According to an aspect of some embodiments of the present invention there is provided an isolated cyclic polypeptide comprising an amino acid consensus sequence X₁X₂X₃X₄X₅X₆X₇X₈X₉X₁₀X₁₁X₁₂X₁₃X₁₄X₁₅X₁₆X₁₇X₁₈, where X₁ and X₁₈ are bonded, X₈ is glycine, X₉ is proline and X₁₀ is glycine, the polypeptide comprising a single internal constraint.

According to an aspect of some embodiments of the present invention there is provided an isolated cyclic polypeptide comprising an amino acid consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃ where X₁ and X₂₃ are bonded, X₁₀ is glycine, X₁₁ is proline and X₁₂ is glycine.

According to an aspect of some embodiments of the present invention there is provided an isolated cyclic polypeptide comprising an amino acid consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂ where X₁ and X₂₂ are bonded, X₁₀ is glycine, X₁₁ is proline and X₁₂ is glycine.

According to some embodiments of the present invention, the amino acids at position 303 and 323 are cysteines.

According to some embodiments of the present invention, the amino acids at position 303 and 322 are cysteines.

According to some embodiments of the present invention, the amino acids at position 301 and 324/325 are cysteines.

According to some embodiments of the present invention, an amino acid at position 312 is glycine, an amino acid at position 313 is proline and an amino acid at position 314 is glycine.

According to some embodiments of the present invention, the cyclic polypeptides comprise a single internal disulfide bond.

According to some embodiments of the present invention, the amino acid at position 315 is arginine lysine or glutamine.

According to some embodiments of the present invention, an amino acid at position 305 is lysine or arginine, and an amino acid at position 307 is isoleucine, leucine or valine and an amino acid at position 309 is isoleucine, leucine, methionine or valine.

According to some embodiments of the present invention, an amino acid at positions 319 and 320 are threonine or alanine.

According to some embodiments of the present invention, the isolated cyclic polypeptide is as set forth in SEQ ID NO: 2 or SEQ ID NO: 27.

According to some embodiments of the present invention, the isolated cyclic polypeptide is as set forth in SEQ ID NO: 31 or SEQ ID NO: 33.

According to some embodiments of the present invention, the isolated cyclic polypeptide is as set forth in SEQ ID NO: 31, 33 or 39.

According to some embodiments of the present invention, is as set forth in SEQ ID NOs: SEQ ID NO: 32, 34, 35 or 36.

According to some embodiments of the present invention, the isolated cyclic polypeptide consists of naturally occurring amino acids.

According to some embodiments of the present invention, the isolated cyclic polypeptides further comprise amino acids of an antigen presenting polypeptide.

According to some embodiments of the present invention, X₁₁ is arginine lysine or glutamine.

According to some embodiments of the present invention, the X₃ is lysine or arginine, and wherein X₅ is isoleucine, leucine or valine and X₇ is isoleucine, leucine, methionine or valine.

According to some embodiments of the present invention, the X₁₅ and X₁₆ are threonine or alanine.

According to some embodiments of the present invention, the X₁ and X₁₉ are cysteines.

According to some embodiments of the present invention, the X₁ and X₁₈ are cysteines.

According to some embodiments of the present invention, the isolated cyclic polypeptides comprise an amino acid sequence of a T-helper epitope.

According to some embodiments of the present invention, the T-helper epitope is a human immunodeficiency virus (HIV) T helper epitope.

According to some embodiments of the present invention, the HIV T-helper epitope comprise amino acids of a C4 domain of HIV gp120.

According to some embodiments of the present invention, the amino acids of a C4 domain of HIV gp120 comprise at least 16 consecutive amino acids of said C4 domain of HIV gp120.

According to some embodiments of the present invention, the at least 16 consecutive amino acids of said C4 domain of HIV gp120 comprise amino acids 421 to 436 of the C4 domain of HIV gp120 according to a numbering in an HXB2 strain.

According to some embodiments of the present invention, the T-helper epitope comprises amino acids of HIV p24 gag.

According to some embodiments of the present invention, the T-helper epitope comprises an amino acid sequence as set forth in SEQ ID NOs: 25 or 26.

According to some embodiments of the present invention, the T-helper epitope is a non-HIV T helper epitope.

According to some embodiments of the present invention, the amino acids of the cyclic peptide are linked C terminal to said T-helper epitope.

According to some embodiments of the present invention, the cyclic peptide is linked via a linker to said T-helper epitope.

According to some embodiments of the present invention, the isolated cyclic polypeptides are no more than 50 amino acids.

According to an aspect of some embodiments of the present invention there is provided a vaccine comprising the polypeptides of the present invention as an active agent and an immunologically acceptable carrier.

According to some embodiments of the present invention the vaccine further comprises an adjuvant.

According to an aspect of some embodiments of the present invention there is provided an article of manufacture comprising the vaccine of the present invention and a CD4 mimic compound.

According to some embodiments of the present invention, the CD4 mimic compound comprises a peptide compound.

According to some embodiments of the present invention, the CD4 mimic compound comprises a small molecule.

According to an aspect of some embodiments of the present invention there is provided a method of generating an immune response against HIV in an individual, the method comprising administering to the individual an effective amount of the vaccine of the present invention, thereby generating the immune response against HIV.

According to some embodiments of the present invention, the method further comprises administering to said individual an effective amount of a CD4 mimic compound.

According to an aspect of some embodiments of the present invention there is provided a method of generating an immune response against HIV in a individual, the method comprising administering to the individual an effective amount of a V3 peptide-based vaccine and further comprising administering to the individual an effective amount of a CD4 mimic compound.

According to some embodiments of the present invention, the administering said CD4 mimic compound is effected following said administering said vaccine.

According to some embodiments of the present invention, the individual is HIV positive.

According to some embodiments of the present invention, the individual is HIV negative.

According to an aspect of some embodiments of the present invention there is provided an isolated polynucleotide comprising a nucleic acid sequence encoding the polypeptides of the present invention.

According to an aspect of some embodiments of the present invention there is provided a nucleic acid construct comprising the isolated polynucleotide of the present invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F are NOESY spectra presenting the aromatic regions of the peptides of the present invention. (FIG. 1A) unconstrained V3_{JRFL}—SEQ ID NO: 8. (FIG. 1B) V3_{T303C,I323C}—SEQ ID NO: 13. (FIG. 1C) V3_{T303C, E322C}—SEQ ID NO: 12. (FIG. 1D) V3_{R304C,G321C}—SEQ ID NO: 11. (FIG. 1E) V3_{K305C,T320C}—SEQ ID NO: 10. (FIG. 1F) V3_{I307C,T319C}—SEQ ID NO: 9 Assignment of cross-peaks representing interactions used in the analysis is shown in color.

FIGS. 2A-B are NOESY spectra presenting amide-amide interactions within the peptides of the present invention: (FIG. 2A) V3_{T303C,I323C}—SEQ ID NO: 13. (FIG. 2B) V3_{I307C,T319C}—SEQ ID NO: 9. Cross-peaks are marked by circles.

FIGS. 3A-C are models of NMR-derived solution structures of the peptides of the present invention: (FIG. 3A) V3_{T303C, E322C}—SEQ ID NO: 12. (FIG. 3B) V3_{R304C,G321C}—SEQ ID NO: 11 and (FIG. 3C) V3_{I307C,T319C}—SEQ ID NO: 9. The disulfide bond is marked in yellow. Side chains are colored accordingly: I307 (only in FIGS. 3A and B) light blue, H308 purple, I309 Green, F317 red and Y318 blue.

FIGS. 4A-C are models of backbone superposition of NMR-derived solution structures of three constrained peptides on the structure of V3_{JRFL} in complex with Fv of 447-52D Ab (blue). Sequence encompassing residues H310-Y318 in each peptide was used for the comparison of: (FIG. 4A) V3_{T303C,E322C}—SEQ ID NO: 12 (green), (FIG. 4B) V3_{R304C,G321C}—SEQ ID NO: 11 (purple) and (FIG. 4C) V3_{I307C,T319C}—SEQ ID NO: 9 (red).

FIG. 5 is a map illustrating the distribution of half-max values for all post-immune sera after the third immunization (post3) with the homologous V3 peptide(black) and gp120(blue) as determined by ELISA. Sera A-D are represented by diamond, square triangle and sphere respectively for each group. Each of the four sera were obtained from rabbits immunized by specific V3 peptides (P1—SEQ ID NO: 1; P2—SEQ ID NO: 2; and P3—SEQ ID NO: 3).

FIGS. 6A-L are binding curves of antibody response of post3 sera for rabbits immunized with linear (P1 A-D)—SEQ ID NO: 1 or constrained C4-V3 peptides (P2 A-D—SEQ ID NO: 2 and P3 A-D—SEQ ID NO: 3). Four rabbits (Rabbits A-D) were tested for each peptide. Binding to the homologues V3 peptide (Black) and gp120 (Blue) are shown; post-immune (triangle), pre-immune (square). Y-axis represents OD-650 nm; X-axis represents the reciprocal of serum dilution.

FIGS. 7A-E are chromatographic and Mass-Spectra (MS) evaluation of synthetic constrained immunogens. Analytical HPLC profile of crude linear (7A), purified linear (7B) and purified cyclic (7C) C4-V3 N301C-G325C (SEQ ID NO: SEQ ID NO: 32). ESI-MS spectrum of purified linear (7D) and purified cyclic (7E) C4-V3 N301C-G325C (SEQ ID NO: 32). The HPLC was run using a 10-60% acetonitrile/water gradient (containing 0.1% TFA) over 20 minutes; Column: Zorbax-Eclipse XDB-C8, 150×4.6 mm; Flow-rate: 1.0 mL/min; Detection at 220 nm; Product Rₜ 11.799 min; The average molecular weight of the linear was 4798.1 while the average molecular weight of the cyclic was 4796.0.

FIGS. 8A-D are graphs illustrating the binding of antibodies elicited by C4-V3 peptides or gp120 to the corresponding V3 peptide and gp120. A) Rabbit B707 serum, immunized with C4-V3L (SEQ ID NO: 1), B) Rabbit B963 serum, immunized with C4-V3_{T303C-E322C} (SEQ ID NO: 31), C) Rabbit B892 immunized with C4-V3_{N301C-G325C} (SEQ ID NO: 32) and D) Rabbit B959 serum, immunized with gp120. Binding to the homologous peptide is shown in triangles (▴ post-immune, Δ pre-immune). Binding to gp120 in is shown in squares (▪ post-immune, □ pre-immune). Y-axis represents OD at 650 nm; X-axis represents the reciprocal of serum dilution. Standard deviation is for duplicates on plate.

FIG. 9 is a graph illustrating the relative cross reactivity of immune sera with gp120 and V3 peptide immunogens. The ratio between the immune-sera binding to gp120 and binding to the homologous peptide used for immunization is represented. The ratio is obtained by dividing the half maximal titer for gp120 by the half maximal titer to the V3 peptide used as immunogen. Shown is the average and standard deviation for each of the four rabbits immunized with each of the peptides. Peptide immunogens are listed by the positions replaced by cysteine according to Table 7. P-value for one sample T-test for a hypothetical mean of 1 is shown above the histograms.

FIG. 10 is a bar graph illustrating the influence of peptide conformation on the binding of immune sera to a cyclic V3 peptide. The binding of the immune-sera to cyclic vs. reduced V3_{T303C/I323C} (SEQ ID NO: 2) is compared to binding to V3L for the C4V3L (SEQ ID NO: 1) and C4-V3_{T303C/I323C} (SEQ ID NO: 2) induced sera. Half-maximal binding titer to cyclic and reduced V3_{T303C/I323C} (SEQ ID NO: 2) and to V3L (SEQ ID NO: 2) with and without DTT was determined by ELISA for each V3_{T303C/I323C} (SEQ ID NO: 2) and V3L (SEQ ID NO: 1) immune serum. In each experiment the binding of the serum to V3_{T303C/I323C} (SEQ ID NO: 2) in the oxidized (dark squares) or reduced state was divided by the binding to V3L (SEQ ID NO: 1). Shown is the average and standard deviation for each of the four rabbits immune sera. P-value for two samples T-test for reduced vs. cyclic is shown.

FIGS. 11A-H are isobolograms plots illustrating synergism between sera of rabbits immunized with C4-V3_{T303C-I323C} (SEQ ID NO: 2) and CD4M33. The concentration of CD4M33 and the serum dilution are plotted on the X-axis and the Y-axis respectively. A line is drawn for the individual serum dilution and CD4M33 concentration when used separately needed for 50% inhibition (black +) and 75% inhibition (gray x). A point representing the serum dilution and CD4M33 concentration in combination needed to achieve the same inhibition is shown. A point below the lines indicates synergy. Serum number and the strain used are indicated on the plot.

### DESCRIPTION OF SPECIFIC EMBODIMENTS OF THE INVENTION

The present invention, in some embodiments thereof, relates to cyclic polypeptides and, uses thereof for the treatment and/or prevention of AIDS.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The third variable region, V3, C296-C331 of the envelope glycoprotein, gp120 of HIV-1 is a target for virus neutralizing antibodies. One such antibody, the 447-52D is a monoclonal antibody that neutralizes diverse strains of the virus. This antibody recognizes the conserved tip of the V3 loop in a β-turn conformation. The present inventors postulated that establishment of the peptide requirements for mimicking the β-hairpin structure of the V3 loop, would allow for the generation of optimal peptide immunogens. Accordingly, the present inventors analyzed various constrained and non-constrained peptides using NMR and showed that the closer the disulfide bond is to the GPGR, the greater the resemblance to a β-hairpin conformation.

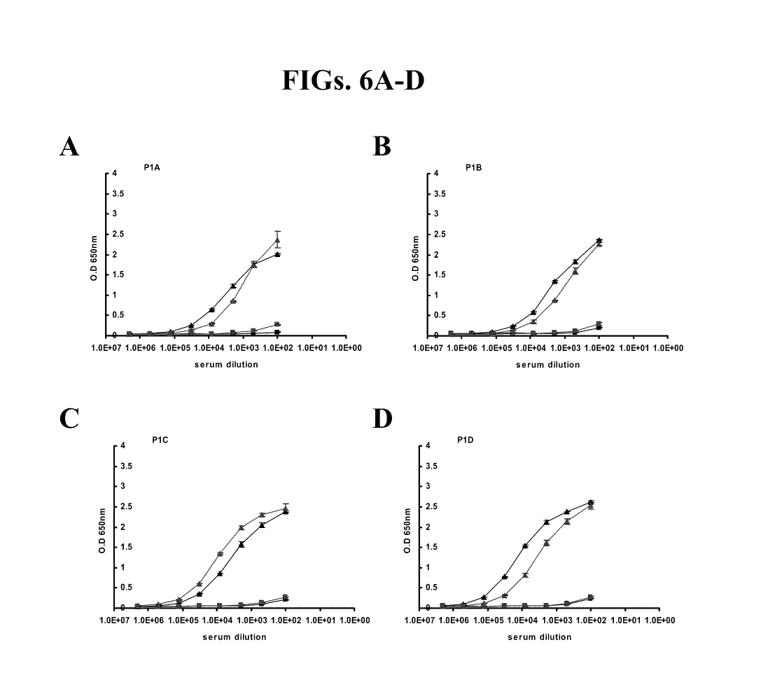

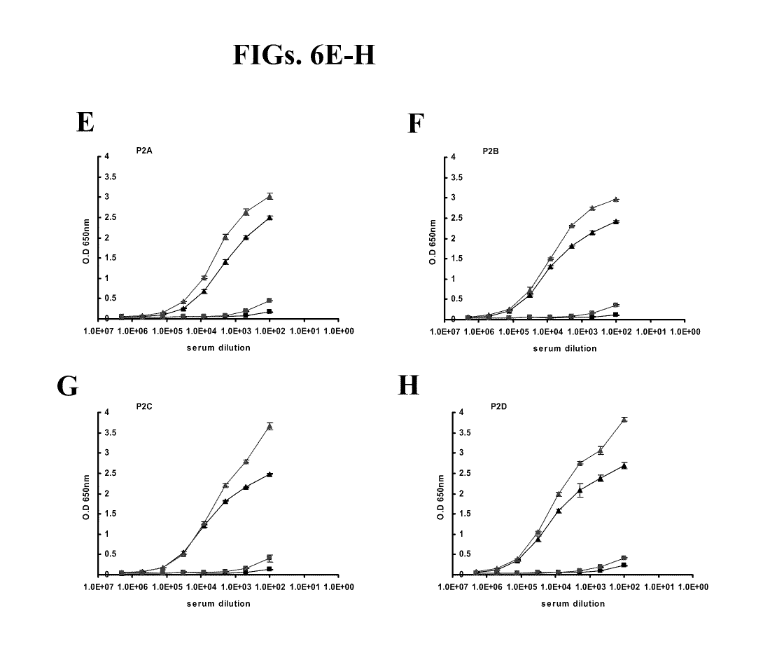

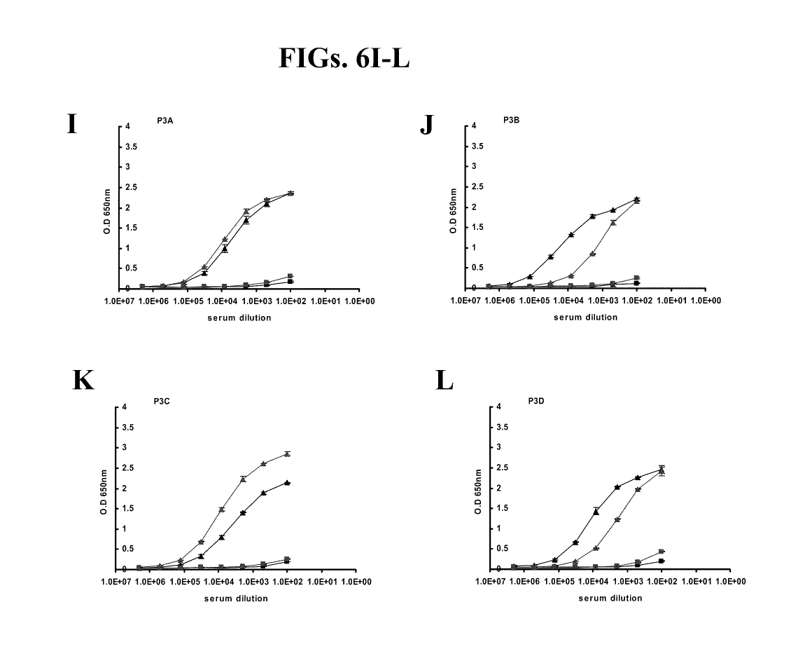

Whilst reducing the present invention to practice, the present inventors initially generated three V3 peptides: a linear peptide (P1, also referred to herein as C4-V3 linear), a peptide constrained by a disulfide bond between residues 303 and 323 (P2, also referred to herein as C4-V3_{T303C-I323C}) and a peptide constrained by a disulfide bond between residues 305 and 320 (P3, also referred to herein as C4-V3_{K303C-T320C}). The present inventors showed that all animals immunized with P2 had high binding to gp120 while for the two other groups the binding was variable and generally lower (FIG. 6A-L). This indicated that P2 immunogen presents the native V3 epitope in a form that is a better mimic of the native V3 conformation.

The sera obtained from rabbits immunized with P2 were able to neutralize five out of seven tested HIV-1 strains (see Table 6, in the Examples section herein below). Much poorer HIV-1 neutralization was obtained by the sera of rabbits immunized with P1 and even worse HIV-1 neutralization was achieved by rabbits immunized with the P3 peptide.

The present inventors thus showed that an optimal immunogen should include the intact V3 epitope recognized by the 447-52D antibody and that cyclization should flank the recognized epitope.

Whilst further reducing the present invention to practice, the present inventors noted that the P2 immunogen comprised an R5B conformation—a particular type of constrained structure postulated to bind to the R5 co-receptor. The present inventors proceeded to synthesize additional cyclized peptides the comprised the intact V3 epitope recognized by the 447-52D antibody ensuring that the cyclization flanked the recognized epitope. Accordingly, the present inventors synthesized a peptide that was constrained at position 303 (as in the P2 immunogen), but this time was constrained to assume an R5A conformation (V3 T303C-E322C). Such a peptide was also able to neutralize an abundance of HIV-1 strains (see Table 9 and Table 10 of the Examples section herein below). By comparing these two peptides with other candidate peptides constrained at positions 301 or 305, the present inventors proved that peptides constrained by a disulfide bond involving residue 303 were more effective immunogens for eliciting sera with gp120 cross-reactivity and neutralization of HIV-1 clade-B isolates.

Thus, according to one aspect of the present invention there is provided an isolated cyclic polypeptide comprising at least nineteen consecutive amino acid residues of a V3 domain of gp120, starting at position 303 and ending at position 323, said positioning being according to a numbering of the V3 domain of gp120 in a HXB2 strain, wherein amino acids at position 303 and 323 are bonded.

The term “polypeptide” as used herein refers to a polymer of natural or synthetic amino acids, encompassing native peptides (either degradation products, synthetically synthesized polypeptides or recombinant polypeptides) and peptidomimetics (typically, synthetically synthesized peptides), as well as peptoids and semipeptoids which are polypeptide analogs, which may have, for example, modifications rendering the peptides even more immunogenic. Such modifications are further described herein below. According to one embodiment, the full length polypeptide is no more than 50 amino acids. According to another embodiment, the full length polypeptide is no more than 100 amino acids. According to another embodiment, the full length polypeptide is no more than 200 amino acids. According to another embodiment, the full length polypeptide is no more than 300 amino acids. According to another embodiment, polypeptide comprises the full length sequence of the gp120. According to yet another embodiment, the polypeptide comprises amino acids of an antigen presenting polypeptide, as further described herein below.

The term “cyclic polypeptide,” as used herein, refers to a polypeptide that comprises an intramolecular covalent bond (e.g. at positions 303 and 323 of the V3 domain of gp120 or at positions 303 and 322 of the V3 domain of gp120).

Cyclization may take place by any means known in the art. The cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the polypeptide may also take place through non-amino acid organic moieties comprised in the polypeptide.

For example, a peptide according to the teachings of the present invention can include at least two cysteine residues flanking the core peptide sequence. In this case, cyclization can be generated via formation of S—S bonds between the two Cys residues. Side chain to side chain cyclization can also be generated via formation of an interaction bond of the formula —(—CH₂—)n-S—CH₂—C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)n-—COOH)—C(R)H—COOH or H—N((CH₂)ₙ—COOH)—C(R)H—NH₂, wherein n=1-4, and further wherein R is any natural or non-natural side chain of an amino acid.

According to one embodiment of this aspect of the present invention the intramolecular covalent bond occurs between two substituted cysteines of the V3 domain of gp120 at positions 303 and 323, such that an internal disulfide bond is generated.

Thus, according to one embodiment, an amino acid sequence of a peptide of this aspect of the present invention is set forth in SEQ ID NO: 27.

As used herein, the term “gp120” refers to an immunodeficiency virus glycoprotein that is typically about 120 kDa in size and corresponding to the 5′ half of the viral Env protein, and containing binding sites for CD4 and chemokine receptors. The third hypervariable domain (V3 domain) of gp120 refers to the 35-37 amino acids of the gp120 which begin at positions 296 and end at positions 331 (numbering according to the HXB2 strain). Exemplary amino acid sequences of V3 domains are set forth in SEQ ID NO: 17 (for the HXB2 strain) and SEQ ID NO: 18 for the JR-FL strain.

According to this aspect of the present invention, the polypeptides of the present invention comprise at least nineteen consecutive amino acid residues of a V3 domain of gp120, starting at position 303 and ending at position 323, (positioning being according to a numbering of the V3 domain of gp120 in a HXB2 strain, as suggested by Ratner et al (22), incorporated herein by reference). It will be appreciated that since some Glade B V3 domains comprise insertion of two amino acids at position 310-311 (e.g. the HXB2 strain), the peptides of the present invention may include 21 consecutive amino acid residues, starting at position 303 and ending at position 323. According to one embodiment, the at least nineteen consecutive residues which start at position 303 and end at position 323 are set forth in SEQ ID NO: 19 or 20.

It will be further appreciated that the polypeptides of this aspect of the present invention may comprise other amino acids from the V3 domain of gp120 apart from those starting at position 303 and ending at position 323. Thus, for example, the polypeptides may comprise amino acids 298-302 from the V3 domain (e.g. as set forth in SEQ ID NO: 21).

As mentioned, the present inventor synthesized an additional peptide, constrained at position 303, but conforming to the R5A conformation. This peptide showed enhanced immunogenicity, neutralizing all 5 tested R5-sensitive strains as well as two X4 strains.

Thus, according to another aspect of the present invention there is provided an isolated cyclic polypeptide comprising at least 18 consecutive amino acids of a V3 domain of gp120, starting at position 303 and ending at position 322, said positioning being according to a numbering of said V3 domain of gp120 in a HXB2 strain, wherein amino acids at position 303 and 322 are bonded. According to this aspect the polypeptide comprises a single internal constraint—i.e. no non-consecutive amino acids (besides 303 and 322) are bonded.

According to one embodiment of this aspect of the present invention the intramolecular covalent bond occurs between two substituted cysteines of the V3 domain of gp120 at positions 303 and 322, such that an internal disulfide bond is generated. Other methods of cyclizing the polypeptide of this aspect of the present invention are described herein above.

Thus, according to one embodiment, an amino acid sequence of a peptide of the present invention is set forth in SEQ ID NO: 33 or SEQ ID NO: 39.

In order to test whether T303 is the optimal position for the disulfide constraint, the present inventors synthesized an additional peptide constrained to assume the same R5B conformation as C4-V3_{T303C-I323C} (SEQ ID NO: 2). The peptide was constrained at positions 301 and 325 and assumed the identical R5B conformation as C4-V3_{T303C-I323C} (SEQ ID NO: 2). In this peptide, the disulfide bond was removed further away from the GPGR loop and the ring size enclosed by the disulfide bond was therefore four-residues larger. In a similar fashion, peptides constrained at positions 301 and 324 would assume an identical R5A conformation as C4-V3_{T303C-E322C} (SEQ ID NO: 31).

Although not as effective as peptides which are constrained at position 303, peptides constrained at position 301 also showed immunogenicity and were capable of neutralizing several R5-sensitive strains.

Thus, according to yet an additional aspect of the present invention there is provided an isolated cyclic polypeptide comprising at least 22 consecutive amino acids of a V3 domain of gp120, starting at position 301 and ending at position 324, said positioning being according to a numbering of said V3 domain of gp120 in a HXB2 strain, wherein amino acids at position 301 and 324 are bonded.

According to still an additional aspect of the present invention there is provided an isolated cyclic polypeptide comprising at least 23 consecutive amino acids of a V3 domain of gp120, starting at position 301 and ending at position 325, said positioning being according to a numbering of said V3 domain of gp120 in a HXB2 strain, wherein amino acids at position 301 and 325 are bonded.

According to this aspect of the present invention, the polypeptide comprises a at least one intramolecular covalent bond (at positions 301 and 324 of the V3 domain of gp120 or at positions 301 and 325 of the V3 domain of gp120.

According to one embodiment of this aspect of the present invention the at least one intramolecular covalent bond occurs between two substituted cysteins of the V3 domain of gp120 at positions 301 and 324, or alternatively at positions 301 and 325, such that an internal disulfide bond is generated. Other methods of cyclizing the polypeptide of this aspect of the present invention are described herein above.

Thus, according to one embodiment, amino acid sequences of a peptide of the present invention is set forth in SEQ ID NOs: 34 and 36.

It will be further appreciated that the polypeptides of this aspect of the present invention may comprise other amino acids from the V3 domain of gp120 apart from those starting at position 301 and ending at position 324/325. Thus, for example, the polypeptides may comprise amino acids 298-300 from the V3 domain (e.g. as set forth in SEQ ID NO: 37).

The polypeptides of the present invention may be fused to or chemically linked with an appropriate carrier molecule, such as tetanus toxin, MLv gp70, cholera toxin, keyhole limpet haemocyanin or gp120. Alternatively, the polypeptides of the present invention may be inserted by genetic engineering techniques into permissible exposed loops of antigenic proteins.

Alternatively the polypeptides of the present invention may be linked to amino acids derived from a T-helper epitope to enhance their immunogenicity.

Exemplary sequences of polypeptides which comprises V3 domain amino acids and T-helper epitope amino acids, according to the present invention are set forth in SEQ ID NO: 2, SEQ ID NO: 31, 32 and 35.

As used herein, the phrase “T-helper epitope” refers to a peptide capable of activating a T helper cell.

The T-helper epitope may be a human immunodeficiency virus (HIV) T helper epitope e.g. from the C4 domain of HIV gp120. According to one embodiment, the T helper epitope comprises about 16 consecutive residues from the C4 domain (about residues 421 to 436—e.g. as set forth in SEQ ID NO: 24. According to another embodiment, the T-helper sequence is a variation of the above, such as that set forth in SEQ ID NO: 25.

Contemplated T helper epitopes from the C4 domain are described in U.S. Pat. Appl. No. 20030147888, incorporated herein by reference. Other T helper determinants from HIV or from non-HIV proteins can also be used. For example, a further T helper epitope suitable for use in the invention is from HIV gag (e.g., residues 262-278). One such sequence, designated GTH1, is as set forth in SEQ ID NO: 22. Variants of this sequence can also be used.

Another contemplated T helper epitope is derived from murine HSP60 458-474 e.g. as set forth in SEQ ID NO: 26.

Alternatively, a carbohydrate such as the outer membrane protein of pneumococcus, or another carbohydrate or protein with immunogenic, T helper activity can be used.

The T-helper epitope amino acids may be linked to the V3 portion of the peptides of the present invention using any method known in the art so long as it does not decrease the immunogenic properties of the peptide.

The amino acids of the V3 domain of gp120 are preferably linked C terminal to the amino acids of the T-helper epitope.

According to one embodiment, the V3 portion of the polypeptide is linked to the T helper epitope via a covalent bond (e.g. a peptide bond). According to another embodiment, the V3 portion of the polypeptide is linked to the T helper epitope via a non-covalent linker. The linkage may be direct or via bonding to an intervening linker element, such as a linker peptide or other chemical moiety, such as an organic polymer.

Exemplary chemical crosslinking methods for conjugating the V3 portion with the T helper epitope portion are described herein below:

### Thiol-Amine Crosslinking:

In this scheme, an amine group of the V3 portion is indirectly conjugated to a thiol group on the T helper portion or vica versa, usually by a two- or three-step reaction sequence. The high reactivity of thiols and their relative rarity in most polypeptides make thiol groups ideal targets for controlled chemical crosslinking. Thiol groups may be introduced into one of the two polypeptides using one of several thiolation methods including SPDP. The thiol-containing biomolecule is then reacted with an amine-containing biomolecule using a heterobifunctional crosslinking reagent.

Amine-Amine Crosslinking:

Conjugation of the V3 portion with the T helper epitope portion can be accomplished by methods known to those skilled in the art using amine-amine crosslinkers including, but not limited to glutaraldehyde, bis(imido esters), bis(succinimidyl esters), diisocyanates and diacid chlorides.

Carbodiimide Conjugation:

Conjugation of the V3 portion with the T helper epitope portion can be accomplished by methods known to those skilled in the art using a dehydrating agent such as a carbodiimide. Most preferably the carbodiimide is used in the presence of 4-dimethyl aminopyridine. As is well known to those skilled in the art, carbodiimide conjugation can be used to form a covalent bond between a carboxyl group of one polypeptide and an hydroxyl group of a second polypeptide (resulting in the formation of an ester bond), or an amino group of a second polypeptide (resulting in the formation of an amide bond) or a sulfhydryl group of a second polypeptide (resulting in the formation of a thioester bond).

Likewise, carbodiimide coupling can be used to form analogous covalent bonds between a carbon group of a first polypeptide and an hydroxyl, amino or sulfhydryl group of a second polypeptide. See, generally, J. March, Advanced Organic Chemistry: Reaction's, Mechanism, and Structure, pp. 349-50 & 372-74 (3d ed.), 1985.

As mentioned herein above, the polypeptides of the present invention may be cloned so that they are expressed in the context of well-characterized fusion proteins. Thus, for example, the polypeptides of the present invention may comprise amino acids of an antigen presenting polypeptide. Exemplary antigen presenting polypeptides include, but are not limited to a thioredoxin polypeptide (see Chakraborty et al., 2006, incorporated herein by reference) and phage polypeptides such as a MuLV polypeptide (see Zolla-Pazner et al., Virology 372, 233-46 (2008), incorporated herein by reference.

As mentioned, the polypeptides of the present invention may comprise modifications. Such modifications include, but are not limited to N terminus modification, C terminus modification, polypeptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α ethylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α thylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α ethylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α ethylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| thylamino)cyclopropane | | | |

It will be appreciated that certain amino acids from the V3 domain described herein above or the T helper epitope domain described herein above may be substituted either conservatively or non-conservatively as further described herein below as long as the substitution does not have a detrimental effect on the immunogenic properties of the polypeptide. Furthermore, the sequence of the V3 can be modified to represent sequences of other clades of HIV-1 such as Glade-A, Glade-C etc.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH₂)₅—COOH]—CO— for aspartic acid. Those non-conservative substitutions which fall under the scope of the present invention are those which still constitute a peptide having immunogenic properties capable of generating antibodies that neutralize the HIV virus.

The present inventors have determined which amino acids in the V3 portion of the polypeptide are the most important for generating such antibodies and as such which amino acids may be replaced by conservative or non-conservative substitutions.

Consider that the V3 portion of the polypeptide of the present invention comprises a consensus sequence as follows:

X₁X₂X₃X₄X₅X₆X₇X₈X₉X₁₀X₁₁X₁₂X₁₃X₁₄X₁₅X₁₆, wherein amino acids on either side of this sequence are bonded (e.g. via an internal disulfide bond). The present inventors regard that a glycine at position X₇ (corresponding to position 312 according to the present HXB2 strain numbering), a proline at position X₈ (corresponding to position 313 according to the present HXB2 strain numbering) and a glycine at position X₉ (corresponding to position 314 according to the present HXB2 strain numbering) are absolutely necessary and cannot be substituted, since these amino acids were found to interact extensively with neutralizing antibodies and to be conserved throughout V3 domains of gp120.

The present inventors also regard the amino acids at positions X2, X₄, X₆, X₁₀, X₁₄ and X₁₅ are also very important since they were also found to interact extensively with neutralizing antibodies. The present inventors therefore believe that these amino acids may only be replaced by conservative amino acid changes.

Therefore, according to another embodiment of this aspect of the invention, X₂ (corresponding to position 305 according to the present HXB2 strain numbering) is lysine or arginine, X₄ (corresponding to position 307 according to the present HXB2 strain numbering) is isoleucine, leucine or valine, X₆ (corresponding to position 309 according to the present HXB2 strain numbering) is isoleucine, leucine, methionine or valine, X₁₄ and X₁₅ (corresponding to position 319 and 320 according to the present HXB2 strain numbering) are threonine or alanine and X₁₀ (corresponding to position 315 according to the present HXB2 strain numbering) is arginine lysine or glutamine.

According to one embodiment of this aspect of the present invention, the polypeptides of the invention comprise the core sequence as set forth in SEQ ID NO: 38.

The following describes the amino acid sequences for individual peptides of the present invention:

1. Consider that the V3 portion of the polypeptide of the present invention comprises a consensus sequence as follows: X₁X₂X₃X₄X₅X₆X₇X₈X₉X₁₀X₁₁X₁₂X₁₃X₁₄X₁₅X₁₆X₁₇X₁₈X₁₉, wherein X₁ are X₁₉ are bonded (e.g. via an internal disulfide bond). The present inventors regard that a glycine at position X₈ (corresponding to position 312 according to the present HXB2 strain numbering), a proline at position X₉ (corresponding to position 313 according to the present HXB2 strain numbering) and a glycine at position X₁₀ (corresponding to position 314 according to the present HXB2 strain numbering) are absolutely necessary and cannot be substituted, since these amino acids were found to interact extensively with neutralizing antibodies and to be conserved throughout V3 domains of gp120.

The present inventors also regard the amino acids at positions X₃, X₅, X₇, X₁₁, X₁₅ and X₁₆ are also very important since they were also found to interact extensively with neutralizing antibodies. The present inventors therefore believe that these amino acids may only be replaced by conservative amino acid changes.

Therefore, according to another embodiment of this aspect of the invention, X₃ (corresponding to position 305 according to the present HXB2 strain numbering) is lysine or arginine, X₅ (corresponding to position 307 according to the present HXB2 strain numbering) is isoleucine, leucine or valine, X₇ (corresponding to position 309 according to the present HXB2 strain numbering) is isoleucine, leucine, methionine or valine, X₁₅ and X₁₆ (corresponding to position 319 and 320 according to the present HXB2 strain numbering) are threonine or alanine and X₁₁ (corresponding to position 315 according to the present HXB2 strain numbering) is arginine lysine or glutamine.

An example of this sequence is set forth in SEQ ID NO: 27.

2. Consider that the V3 portion of the polypeptide of the present invention comprises a consensus sequence as follows: X₁X₂X₃X₄X₅X₆X₇X₈X₉X₁₀X₁₁X₁₂X₁₃X₁₄X₁₅X₁₆X₁₇X₁₈, wherein X₁ are X₁₈ are bonded (e.g. via an internal disulfide bond). The present inventors regard that a glycine at position X₈ (corresponding to position 312 according to the present HXB2 strain numbering), a proline at position X₉ (corresponding to position 313 according to the present HXB2 strain numbering) and a glycine at position X₁₀ (corresponding to position 314 according to the present HXB2 strain numbering) are absolutely necessary and cannot be substituted, since these amino acids were found to interact extensively with neutralizing antibodies and to be conserved throughout V3 domains of gp120.

The present inventors also regard the amino acids at positions X₃, X₅, X₇, X₁₁, X₁₅ and X₁₆ are also very important since they were also found to interact extensively with neutralizing antibodies. The present inventors therefore believe that these amino acids may only be replaced by conservative amino acid changes.

Therefore, according to another embodiment of this aspect of the invention, X₃ (corresponding to position 305 according to the present HXB2 strain numbering) is lysine or arginine, X₅ (corresponding to position 307 according to the present HXB2 strain numbering) is isoleucine, leucine or valine, X₇ (corresponding to position 309 according to the present HXB2 strain numbering) is isoleucine, leucine, methionine or valine, X₁₅ and X₁₆ (corresponding to position 319 and 320 according to the present HXB2 strain numbering) are threonine or alanine and X₁₁ (corresponding to position 315 according to the present HXB2 strain numbering) is arginine lysine or glutamine.

An example of this sequence is set forth in SEQ ID NO: 39.

3. Consider that the V3 portion of the polypeptide of the present invention comprises a consensus sequence as follows: X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃, wherein X₁ are X₂₃ are bonded (e.g. via an internal disulfide bond). The present inventors regard that a glycine at position X₁(O)(corresponding to position 312 according to the present HXB2 strain numbering), a proline at position X₁₁ (corresponding to position 313 according to the present HXB2 strain numbering) and a glycine at position X₁₂ (corresponding to position 314 according to the present HXB2 strain numbering) are absolutely necessary and cannot be substituted, since these amino acids were found to interact extensively with neutralizing antibodies and to be conserved throughout V3 domains of gp120.

The present inventors also regard the amino acids at positions X₅, X₇, X₉, X₁₃, X₁₇ and X₁₈ are also very important since they were also found to interact extensively with neutralizing antibodies. The present inventors therefore believe that these amino acids may only be replaced by conservative amino acid changes.

Therefore, according to another embodiment of this aspect of the invention, X₅ (corresponding to position 305 according to the present HXB2 strain numbering) is lysine or arginine, X₇ (corresponding to position 307 according to the present HXB2 strain numbering) is isoleucine, leucine or valine, X₉ (corresponding to position 309 according to the present HXB2 strain numbering) is isoleucine, leucine, methionine or valine, X₁₇ and X₁₈ (corresponding to position 319 and 320 according to the present HXB2 strain numbering) are threonine or alanine and X₁₃ (corresponding to position 315 according to the present HXB2 strain numbering) is arginine lysine or glutamine.

An example of this sequence is set forth in SEQ ID NO:34.

4. Consider that the V3 portion of the polypeptide of the present invention comprises a consensus sequence as follows: X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂, wherein X₁ are X₂₂ are bonded (e.g. via an internal disulfide bond). The present inventors regard that a glycine at position X₁₀ (corresponding to position 312 according to the present HXB2 strain numbering), a proline at position X₁₁ (corresponding to position 313 according to the present HXB2 strain numbering) and a glycine at position X₁₂ (corresponding to position 314 according to the present HXB2 strain numbering) are absolutely necessary and cannot be substituted, since these amino acids were found to interact extensively with neutralizing antibodies and to be conserved throughout V3 domains of gp120.

The present inventors also regard the amino acids at positions X₅, X₇, X₉, X₁₃, X₁₇ and X₁₈ are also very important since they were also found to interact extensively with neutralizing antibodies. The present inventors therefore believe that these amino acids may only be replaced by conservative amino acid changes.

Therefore, according to another embodiment of this aspect of the invention, X₅ (corresponding to position 305 according to the present HXB2 strain numbering) is lysine or arginine, X₇ (corresponding to position 307 according to the present HXB2 strain numbering) is isoleucine, leucine or valine, X₉ (corresponding to position 309 according to the present HXB2 strain numbering) is isoleucine, leucine, methionine or valine, X₁₇ and X₁₈ (corresponding to position 319 and 320 according to the present HXB2 strain numbering) are threonine or alanine and X₁₃ (corresponding to position 315 according to the present HXB2 strain numbering) is arginine lysine or glutamine.

An example of this sequence is set forth in SEQ ID NO:36.

The polypeptides of the present invention may be protected by functional groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that enhance immunogenicity of the peptides.

These moieties can be cleaved in vivo, either by hydrolysis or enzymatically, inside the cell. Hydroxylprotecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH₃—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

The peptides according to the present invention can further include salts and chemical derivatives of the peptides. As used herein, the phrase “chemical derivative” describes a polypeptide of the invention having one or more residues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. Also included as chemical derivatives are those peptides that contain one or more naturally occurring amino acid derivatives of the twenty standard amino acids. The chemical derivatization does not comprehend changes in functional groups which change one amino acid to another.

It will be appreciated that since one of the main obstacles in using short peptide fragments in therapy is their proteolytic degradation by stereospecific cellular proteases, the peptides of the present invention preferably comprise at least one D-isomer of natural amino acids [i.e., inverso peptide analogues, Tjernberg (1997) J. Biol. Chem. 272:12601-5].

The polypeptides of the present invention can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Solid-phase peptide synthesis may be initiated from the C-terminus of the peptide by coupling a protected alpha-amino acid to a suitable resin. Such a starting material can be prepared by attaching an .alpha.-amino-protected amino acid by an ester linkage to a chloromethylated resin or to a hydroxymethyl resin, or by an amide bond to a BHA resin or MBHA resin. The preparation of the hydroxymethyl resin is described by Bodansky et al., Chem. Ind., 38:1597-1598 (1966). Chloromethylated resins are commercially available. The preparation of such a resin is described by Stewart et al. (Solid Phase Peptide Synthesis, Freeman & Co., San Francisco 1969, chapter 1, 1-6). BHA and MBHA resin supports are commercially available and are generally used only when the desired peptide being synthesized has an unsubstituted amide at the C-terminus. Coupling methods involving the use of a coupling agents such as N,N′ dicyclohexylcarbodiimide or N,N′-diisopropylcarbodiimide and others are well-known in the art. See, for example, Gross et al., The Peptides: Analysis, Structure, Biology, Vol. I, Academic Press, 1979, the disclosure of which is hereby incorporated by reference.

The alpha-amino group of each amino acid employed in the peptide synthesis must be protected during the coupling reaction to prevent side reactions involving their active .alpha.-amino function. Certain amino acids have reactive side-chain functional groups (e.g., sulfhydryl, amino, carboxyl, and hydroxyl) that must also be protected with suitable protecting groups to prevent a chemical reaction from occurring during the initial and subsequent coupling steps. In selecting a particular protecting group, the following general rules are typically followed. An alpha-amino protecting group should render the alpha-amino function inert under the conditions of the coupling reaction, should be readily removable after the coupling reaction under conditions that do not remove side-chain protecting groups nor alter the structure of the peptide, and should substantially reduce the possibility of racemization upon activation, immediately prior to coupling.

Side-chain protecting groups should render the side chain functional group inert under the conditions of the coupling reaction, should be stable under the conditions employed to remove the alpha-amino protecting group, and should be readily removable from the fully-assembled peptide under conditions that do not alter the peptide chain's structure.

Conventional protecting groups include 2-(p-biphenyl)isopropyloxycarbonyl; t-butyloxycarbonyl (BOC), fluorenylmethyloxycarbonyl (FMOC), t-amyloxycarbonyl, adamantyl-oxycarbonyl, and p-methoxybenzyloxycarbonyl, benzyloxycarbonyl (CBZ), substituted CBZ, such as, e.g., p-chlorobenzyloxycarbonyl, p-nitrobenzyloxycarbonyl, p-bromobenzyloxycarbonyl, and p-methoxybenzyloxycarbonyl, o-chlorobenzyloxycarbonyl, 2,4-dichlorobenzyloxycarbonyl, 2,6-dichlorobenzyloxycarbonyl, and the like; cycloalkyloxycarbonyl, and isopropyloxycarbonyl. It is known that such groups vary in reactivity with the agents employed for their removal. See, for example, Gross et al., The Peptides: Analysis, Structure, Biology, Vol. 3, Academic Press, 1981 (incorporated by reference in its entirety). The preferred .alpha.-amino protecting groups are tBOC and FMOC. Other standard .alpha.-amino group de-protecting reagents, such as HCl in dioxane, and conditions for the removal of specific .alpha.-amino protecting groups are well-known in the art, e.g., Lubke et al., Chemie und Biochemie der Aminosauren, Peptide und Proteine I, Chapter II-1, 102-117 (Georg Thieme Verlag Stuttgart 1975. incorporated by reference in its entirety).

An alternative to the stepwise approach is the fragment condensation method in which pre-formed peptides of shorter length, each representing part of the desired sequence, are coupled to a growing chain of amino acids bound to a solid phase support. For this stepwise approach, a particularly suitable coupling reagent is N,N′-dicyclohexyl-carbodiimide or diisopropylcarbodiimide. The selection of the coupling reagent, as well as the choice of the fragmentation pattern needed to couple fragments of the desired nature and size are important for success and are known to those skilled in the art.

In appropriate circumstances and when certain structural requirements of the polypeptide are met, when it is desired to cleave the polypeptide without removing protecting groups, the protected peptide-resin can be subjected to methanolysis, thus yielding a protected peptide with a methylated C-terminal carboxyl group. This methyl ester can be hydrolyzed under mild alkaline conditions to give the free carboxyl group. Protecting groups on the peptide chain can then be removed by treatment with a strong acid, such as liquid hydrogen fluoride. See, for example, Moore et al., In Peptides, Proc. Fifth Amer. Pept. Symp., 518-521 (Goodman et al., eds., 1977).

Purification of the cyclic polypeptides of the invention is typically achieved using chromatographic techniques, such as preparative HPLC including reverse phase TALC, or gel permeation, ion exchange, partition and/or affinity chromatography.

Recombinant techniques may also be used to generate the polypeptides of the present invention. To produce a polypeptide of the present invention using recombinant technology, a polynucleotide (e.g. as set forth in SEQ ID NO: 23 or SEQ ID NO: 28) encoding the polypeptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter (Zabala M, et al., Cancer Res. 2004, 64(8): 2799-804).

The nucleic acid construct (also referred to herein as an “expression vector”) of the present invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase RNA stability [Soreq et al., 1974; J. Mol. Biol. 88: 233-45).

Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMT010/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

It will be appreciated that the polypeptides of the present invention may be expressed directly in the subject (i.e. DNA vaccination in vivo gene therapy) or may be expressed ex vivo in a cell system, as described herein above (autologous or non-autologous) and then administered to the subject.

Recombinant viral vectors are useful for in vivo expression of the polypeptides of the present invention since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. Optionally, the construct may also include a signal that directs polyadenylation, as well as one or more restriction sites. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription of the inserted coding sequence, the expression construct of the present invention can also include sequences engineered to enhance stability, production, purification, yield or toxicity of the expressed RNA.

In addition to being synthesizable in host cells, the polypeptide of the present invention can also be synthesized using in vitro expression systems. These methods are well known in the art and the components of the system are commercially available.

Following synthesis, the polypeptides of the present invention may optionally be tested for their immunogenicity and ability to neutralize HIV.

To determine the immunogenicity of the polypeptides of the present invention, the antibody response of the recipient (e.g. test animal) is typically measured by obtaining a serum sample at appropriate intervals in the immunization schedule and testing it for antibodies that (a) bind the polypeptide, gp120, HIV-1 virions and/or infected cells, and/or (2) neutralize the virus. Binding assays for anti-HIV-1 antibodies are conventional and are described in detail in many of the references cited herein. HIV-1 neutralization assays are also well known in the art, and exemplary description may be found in Mascola J R et al. (2002) J. Virol. 76:4810-21; Montefiori D C et al. (1988) J Clin Microbiol 26:231-235; and D'Souza M P et al. (1997) J. Infec. Dis. 175:1056-62.

The polypeptides of the present invention may be used generate an immune response against HIV in an individual (humans or other animals).

Although the immunogenic polypeptides of the present invention is typically administered to individuals that are not infected with HIV (e.g. those at risk of infection), HIV-negative, they also may be administered to individuals who are infected with the virus, HIV-positive (e.g. AIDS patients) in an effort to alter the immune response to the virus e.g. by inducing a neutralizing antibody response or any other accompanying protective form of immune reactivity. Also provided is a method for inhibiting viral infection or spread of virus by exploiting the co-receptor specificity of the peptide of the present invention. Accordingly, the polypeptides of the present invention may be used to treat individuals who are HIV positive.

While further reducing the present invention to practice, the present inventors noted that soluble CD4 is capable of causing a conformational change in gp120 that exposes the V3, thereby broadening the neutralization profile of anti-V3 antibodies to include neutralization of neutralization-resistant viruses in which the V3 is occluded. The present inventors showed that CD4 mimic compounds are capable of broadening the neutralization profile of antibodies elicited by the peptide immunogens of the present invention in a synergistic fashion.

The present inventors suggest the use of pre-exposure prophylactic administration of CD4 mimic compounds to vaccinated individuals or post-exposure administration of CD4-mimic compounds to vaccinated individuals. Preferably, the CD4 mimic compounds should be given in proximity to the time of infection in order to broaden the potency of the V3-based vaccine (e.g. up to 48-hours post exposure).

The present inventors postulate that upon interaction with the virus the CD4-mimic compound will bind to the viral gp120 and induce conformational changes in this protein that will expose the V3 before the virus can attach to target cells. The existing V3-directed antibodies could then bind to the virus, unimpeded by the steric hindrance manifested when HIV-1 binds to cell-surface CD4, thus preventing potential infection of cells carrying the CCR5 and the CXCR4 co-receptors.

Thus, according to another aspect of the present invention, there is provided q method of generating an immune response against HIV in an individual, the method comprising administering to the individual an effective amount of a V3 peptide-based vaccine and further comprising administering to the individual an effective amount of a CD4 mimic compound.

As used herein, the phrase “V3 peptide based vaccine” refers to a vaccine which comprises a peptide which comprises at least 5 amino acids (and more preferably at least 10 amino acids) from the third hypervariable loop (V3) domain of the surface subunit of the envelope glycoprotein (gp120) of HIV-1.

According to one embodiment, the V3 peptide based vaccine comprises a cyclized peptide.

According to another embodiment, the V3 peptide based vaccine comprises the peptides of the present invention.

As used herein, the phrase “CD4 mimic compound” refers to a compound that is capable of causing a conformational change in the gp120 polypeptide that exposes the V3 domain.

According to one embodiment, the CD4 mimic compound is a peptide. Examples of peptide CD4 mimic compounds are described in Arthos J, Cicala C, Steenbeke T D, Chun T W, Dela Cruz C, et al. (2002) J Biol Chem 277: 11456-11464; Allaway G P, Davis-Bruno K L, Beaudry G A, Garcia E B, Wong E L, et al. (1995) AIDS Res Hum Retroviruses 11: 533-539. Trkola A, Pomales A B, Yuan H, Korber B, Maddon P J, et al. (1995) J Virol 69: 6609-6617; Martin L, Stricher F, Misse D, Sironi F, Pugniere M, et al. (2003) Nat Biotechnol 21: 71-76, all of which are incorporated herein by reference.

According to another embodiment, the CD4 mimic compound is a small molecule compound. Examples of small-molecule CD4 mimics include the N-phenyl-N′-(2,2,6,6-tetramethyl-piperidin-4-yl)-oxalamide analogs NBD-556 and NBD-557 and their derivatives, and others disclosed in Scholl A, Madani N, Klein J C, Hubicki A, Ng D, et al. (2006) Biochemistry 45: 10973-10980; Zhao Q, Ma L, Jiang S, Lu H, Liu S, et al. (2005) Virology 339: 213-225, incorporated herein by reference. Although the CD4 mimic compounds are typically administered at a time very close to known exposure to the HIV virus—e.g. no more than 48 hours following exposure and preferably no more than 24 hours, the present invention also contemplates administration of the CD4 mimic compounds at other time periods.

The CD4 mimic compounds may be available as an article of manufacture together with the peptide vaccines. The article of manufacture may also comprise instructions for use. Preferably the CD4 mimic compounds are packaged separately the peptide vaccines.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. It will be appreciated that the treating may be performed alone or in conjunction with other therapies.

The polypeptides of the present invention may be administered per se, or alternatively, as part of a composition i.e. vaccine, which comprises an immunologically acceptable carrier. It will be appreciated that the polypeptides of the present invention may be active per se, or may act as “pro-drugs” that are converted in vivo to the active form, e.g., proteolytic cleavage. It will be appreciated that the polypeptides may be administered in the form an expression construct which comprises the corresponding nucleic acid sequence to the polypeptide. The expression construct may be administered instead of the polypeptides themselves (e.g. in a prime boost protocol) or in addition to the polypeptides of the present invention.

General methods to prepare immunogenic or vaccine compositions are described in Remington's Pharmaceutical Science; Mack Publishing Company Easton, Pa. (latest edition). To increase immunogenicity, the polypeptides of the present invention may be adsorbed to or conjugated to beads such as latex or gold beads, ISCOMs, and the like. Immunogenic compositions may comprise adjuvants, which are substance that can be added to an immunogen or to a vaccine formulation to enhance the immune-stimulating properties of the immunogenic moiety. Liposomes are also considered to be adjuvants (Gregoriades, G. et al., Immunological Adjuvants and Vaccines, Plenum Press, New York, 1989) Examples of adjuvants or agents that may add to the effectiveness of proteinaceous immunogens include aluminum hydroxide, aluminum phosphate, aluminum potassium sulfate (alum), beryllium sulfate, silica, kaolin, carbon, water-in-oil emulsions, and oil-in-water emulsions. A preferred type of adjuvant is muramyl dipeptide (MDP) and various MDP derivatives and formulations, e.g., N-acetyl-D-glucosaminyl-(.beta.1-4)-N-acetylmuramyl-L-alanyl-D-isoglutami-ne (GMDP) (Hornung, R L et al. Ther Immunol 1995 2:7-14) or ISAF-1 (5% squalene, 2.5% pluronic L121, 0.2% Tween 80 in phosphate-buffered solution with 0.4 mg of threonyl-muramyl dipeptide; see Kwak, L W et al. (1992) N. Engl. J. Med., 327:1209-1238). Other useful adjuvants are, or are based on, cholera toxin, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives such as QS21 (White, A. C. et al. (1991) Adv. Exp. Med. Biol., 303:207-210) which is now in use in the clinic (Helling, F et al. (1995) Cancer Res., 55:2783-2788; Davis, T A et al. (1997) Blood, 90: 509), levamisole, DEAE-dextran, blocked copolymers or other synthetic adjuvants. A number of adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.), Amphigen (oil-in-water), Alhydrogel (aluminum hydroxide), or a mixture of Amphigen and Alhydrogel. Aluminum is approved for human use.

The present invention also contemplates therapeutic compositions and methods comprise antibodies or an antiserum induced in one subject using the peptides of the present invention, removed from that subject and used to treat another subject by passive immunization or transfer of the antibodies. This is particularly useful for treating neonates exposed to maternal virus, healthcare workers immediately after acute exposure to HIV-1 through patient contact or material handling, or shortly after primary exposure to HIV-1 through sexual contact. For disclosure of such passive immunization with patient sera, neutralizing antisera or mAbs, see Nishimura Y et al. (2003) Proc Natl Acad Sci USA 100:15131-36; Mascola J R (2003) Curr Mol. Med. 3:209-16; Ferrantelli F et al. (2003) AIDS 17:301-9; Ferrantelli F et al (2002) Curr Opin Immunol. 14:495-502; Xu W et al. (2002) Vaccine 20:1956-60; Nichols C N et al. (2002) AIDS Res Hum Retrovir. 8:49-56; Cho M W et al. (2000) J. Virol. 74:9749-54; Mascola J R et al. (2000) Nat. Med. 6:207-10; Andrus. L et al. (1998) J. Inf. Dis. 77: 889-897; Parren P W (1995) AIDS 9:F1-6; Hinkula J et al. (1994) J Acquir Immune Defic Syndr. 7:940-51; Prince A M et al. (1991) AIDS Res Hum Retrovir 7:971-73; Emini E A et al. (1990) J. Virol. 64:3674-84, all incorporated by reference.

The amount of active agent to be administered depends on the precise peptide selected, the health and weight of the recipient, the route of administration, the existence of other concurrent treatment, if any, the frequency of treatment, the nature of the effect desired, and the judgment of the skilled practitioner.

An exemplary dose for treating a subject is an amount of up to about 100 milligrams of active polypeptide per kilogram of body weight. A typical single dosage of the polypeptide or chimeric protein is between about 1 ng and about 100 mg/kg body weight, and preferably from about 10 μg to about 50 mg/kg body weight. A total daily dosage in the range of about 0.1 milligrams to about 7 grams is preferred for intravenous administration. A useful dose of an antibody for passive immunization is between 10-100 mg/kg. It has been suggested (see references cited above for passive immunity) that an effective in vivo dose of an antibody/antiserum is between about 10- and 100-fold more than an effective neutralizing concentration or dose in vitro. These dosages can be determined empirically in conjunction with the present disclosure and state-of-the-art. The polypeptides of the present invention may be administered alone or in conjunction with other therapeutics directed to the treatment of the disease or condition.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized.

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of vaccine to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Pharmaceutically acceptable acid addition salts of certain compounds of the invention containing a basic group are formed where appropriate with strong or moderately strong, non-toxic, organic or inorganic acids by methods known to the art. Exemplary of the acid addition salts that are included in this invention are maleate, fumarate, lactate, oxalate, methanesulfonate, ethanesulfonate, benzenesulfonate, tartrate, citrate, hydrochloride, hydrobromide, sulfate, phosphate and nitrate salts. Pharmaceutically acceptable base addition salts of compounds of the invention containing an acidic group are prepared by known methods from organic and inorganic bases and include, for example, nontoxic alkali metal and alkaline earth bases, such as calcium, sodium, potassium and ammonium hydroxide; and nontoxic organic bases such as triethylamine, butylamine, piperazine, and tri(hydroxymethyl)methylamine. The polypeptides of the invention may be incorporated into convenient dosage forms, such as capsules, impregnated wafers, tablets or preferably injectable preparations. Solid or liquid pharmaceutically acceptable carriers may be employed. Preferably, the peptides of the invention are administered systemically, e.g., by injection or infusion. Administration may be by any known route, preferably intravenous, subcutaneous, intramuscular, intrathecal, intracerebroventricular, or intraperitoneal. (Other routes are noted below) Injectables can be prepared in conventional forms, either as solutions or suspensions, solid forms suitable for solution or suspension in liquid prior to injection, or as emulsions. To enhance delivery or immunogenic activity, the peptides of the present invention can be incorporated into liposomes using methods and compounds known in the art.

The pharmaceutical preparations are made following conventional techniques of pharmaceutical chemistry. The pharmaceutical compositions may also contain minor amounts of nontoxic auxiliary substances such as wetting or emulsifying agents, pH buffering agents and so forth. The peptides are formulated using conventional pharmaceutically acceptable parenteral vehicles for administration by injection. These vehicles are nontoxic and therapeutic, and a number of formulations are set forth in Remington's Pharmaceutical Sciences, Gennaro, 18th ed., Mack Publishing Co., Easton, Pa. (1990)). Nonlimiting examples of excipients are water, saline, Ringer's solution, dextrose solution and Hank's balanced salt solution. Formulations according to the invention may also contain minor amounts of additives such as substances that maintain isotonicity, physiological pH, and stability. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension. Optionally, a suspension may contain stabilizers.

The peptides and other useful compositions of the invention are preferably formulated in purified form substantially free of aggregates and other protein materials, preferably at concentrations of about 1.0 ng/ml to 100 mg/ml.

As noted above, therapeutic compositions of the invention may comprise, in addition to the peptides, analogues, isosteres, mimics, chimeric proteins or cyclic peptides, one or more additional anti-HIV agents, such as protease inhibitors or reverse transcriptase inhibitors as well as immunostimulatory agents including cytokines such as interferons or interleukins. In fact, pharmaceutical compositions comprising any known HIV therapeutic in combination with the compounds disclosed herein are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents. Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Constrained peptides with cysteine residues at positions 303 and 305

Materials and Methods

Peptide Synthesis

Two methods of peptide synthesis were used as follows:

1. On 443A peptide synthesizer (Applied Biosystems) using 0.1 mM fast fluorenylmethoxycarbonyl (Fmoc) chemistry. First 22 amino acids were attached using single coupling, the rest of peptide was assembled using double coupling. Capping of un-reacted amino groups with acetic anhydride was used at every step. To a solution of 90% pure linear peptide in ammonium acetate buffer a solution of potassium ferricyanide was added and the resulting solution was stirred at room temperature overnight. The progress of the reaction was judged using MS analysis. The final reaction solution was purified to ˜90% by HPLC on a Waters C18 Delta Pak column using acetonitrile/water gradient in 0.1% TFA. All protected amino acids and coupling reagents were purchased from Novabiochem (Laufelfingen, Switzerland). Synthesis grade solvents were obtained from Labscan (Dublin, Ireland).

2. Conventional solid-phase peptide synthesis, using an ABIMED AMS-422 automated solid-phase multiple peptide synthesizer (Langenfeld, Germany) and 9-(Fmoc) protection at the α-amine. A Wang resin, loaded with the N-Fmoc protected C-terminal amino acid (25 μmol, 0.55-0.76 mmol/g resin) was used in each reaction vessel. Side chain protections were Lys(t-Boc), Asn(Trt), Gln(Trt) His(Trt), Asp(O-t-But), Glu(O-t-But), Ser(t-But), Tyr(t-But), Thr(t-But) and Arg(Pbf). Double coupling of each residue was carried out in dimethylformamide with 4 equivalents of each N-Fmoc amino acid, 4 equivalents of benzotriazole-1-yl-oxy-tris-pyrrolidino-phosphonium hexafluorophosphate reagent, and 8 equivalents of 4-methyl-morpholine for 20-45 minutes at room temperature.

Cleavage of the peptide was performed by reacting the peptide-resins with 1.8 ml trifluoroacetic acid cocktail [TFA/H₂O/triethylsilane (90/5/5, v:v:v)] for 2 hours at room temperature. Peptides that contain Cys or Met were treated with TFA/H₂O/triethylsilane/thioanisole (85/5/5/5 v:v:v:v); The cleaved peptides were precipitated and washed with ice-cold di-tert-butylether, recovered by centrifugation, dissolved in water, and lyophilized. Cyclization of peptides was performed in 0.1 N ammonium acetate buffer (pH 7-8) in high dilution (1 mg/2-3 ml). 100 μl aliquots of a solution of potassium ferricyanide (50 mg in 30 ml water) were added to accelerate the cyclization until the yellow color persists and the solution was stirred overnight to allow complete oxidation. Disulfide bond formation was monitored by analytical HPLC and judged to have occurred by reaction with Ellman's reagent and by mass spectroscopy: a 2 Da decrease in the molecular weight of cyclic product compared to its linear precursor. All the peptides were purified by preparative reverse-phase HPLC using a Vydac reverse phase C4 or C18 column, 22×250 mm, and water/acetonitrile/0.1% TFA gradients. The yields of the cyclization reactions were 20-25%. HPLC-purified peptides were analyzed by time-of-flight mass spectrometry using a VG MALDI TOF mass spectrometer (VG Fisons, Altrincham, U.K.), and were found to exhibit the calculated mass. Final peptides subjected to NMR analysis were greater than 95% homogeneous as judged by analytical HPLC. The nomenclature used in this paper reflects the positions of the cysteine substitutions used to form the disulfide bond and is indicated in the second column of Table 3, herein below.

TABLE 3
Immmunogenic peptides

| | |
|---|---|
| P1 C4-V3 linear | KQIINMWQEVGKAMYA- |
| | RPNNNTRKSIHIGPGRAFYTTGEI (SEQ ID NO: 1) |
| P2 C4-V3 T303C-1323C | KQIINMWQEVGKAMYA- |
| | RPNNNCRKSIHIGPGRAFYTTGEC (SEQ ID NO: 2) |
| P3 C4-V3 K305C-T320C | KQIINMWQEVGKAMYA- |
| | RPNNNTRCSIHIGPGRAFYTCGEI_(SEQ ID NO: 3) |

Peptides used for ELISA

| | |
|---|---|
| 2124 P1 homolog | Biotin-GSGTRKSIHIGPGRAFYTTGEI (SEQ ID NO: 4) |
| 2125 P2 homolog | Biotin-GSGCRKSIHIGPGRAFYTTGEC (SEQ ID NO: 5) |
| 2129 P3 homolog | Biotin-GSGTRCSIHIGPGRAFYTCGEI (SEQ ID NO: 6) |
| 2135 C4 homolog | Biotin-GSGKQIINMWQEVGKAMYA (SEQ ID NO: 7) |

Peptides used for NMR

| | |
|---|---|
| V3 linear JRFL | TRKSIHIGPGRAFYTTGEI (SEQ ID NO: 8) |
| V3 I307C, T319C | TRKSCHIGPGRAFYCTGEI (SEQ ID NO: 9) |
| V3 K305C, T320C | RRCSIHIGPGRAFYTCGER (SEQ ID NO: 10) |
| V3 R304C, G321C | TCKSIHIGPGRAFYTTCE (SEQ ID NO: 11) |
| V3 T303C, E322C | CRKSIHIGPGRAFYTTGC (SEQ ID NO: 12) |
| V3 T303C, I323C | CRKSIHIGPGRAFYTTGEC (SEQ ID NO: 13) |

Underlined R residues indicate non-native residues added to increase solubility

The numbering system used for the V3 peptides follows that suggested by Ratner et al (22).

For ELISA binding measurements peptides containing the V3 segment without the C4 segment were synthesized. Biotin was coupled to the N-terminus of the peptides on the resin, using identical conditions to those used to couple Fmoc amino acids to the growing chain. An SGS sequence was added as a spacer between the V3 binding epitope and the biotin.

NMR Sample Preparation

Constrained V3 peptides (Table 3, herein above) were dissolved in a solution of 10 mM NaH₂PO₄ buffer containing 95% H₂O/5% D₂O, pH 6.0, and 0.05% NaN₃ at concentration ranging between 0.65-2 mM. Sample volumes were adjusted to 350 μl and the samples were placed into a Shigemi NMR test tube.

NMR Measurements

All NMR spectra were acquired on a Bruker DRX 800 MHz spectrometer using a triple resonance inverse either RT or cryo probes, equipped with gradients. All measurements were performed at 273.5-277 K. The pulse sequence of the 2D HOHAHA (23) measurements used a WALTZ (24) or DIPSI-2 (25) sequence for isotropic mixing. The DQF-COSY spectra were acquired according to standard procedures (26). Water suppression schemes used in our NMR experiments included either WATERGATE, 3-9-19 or excitation sculpting sequences (27-29). Mixing times used for the NOESY experiments were 400 msec for V3_{T303C,E322C}, 200 msec for V3_{T303C,I323C}, 250 msec for V3_{R304C,G321C}, 45-200 msec for V3_{K305C,T320C} and 200 msec for V3_{I307C,T319C}. The spectra were processed using the NMRPipe (30) and XWIN-NMR (Bruker BioSpin DE) software. All data were analyzed using the NMRView (31) and the AURELIA software packages (Bruker BioSpin DE) (32).

Structure Calculation

Sequential assignment was performed using the procedure outlined by Wiithrich (33) and was later used as input data for the automated NOE assignment and structure calculation steps. ³J_{HNHα} coupling constants were determined from the DQF-COSY spectrum, by the measurement of the separation between Lorentzian-fitted anti-phase doublets using AURELIA (Bruker BioSpin DE).

Structure calculations of the V3_{T303C,E322C}, V3_{R304C,G321C} and V3_{I307C,T319C} peptides were performed using the CYANA 2.0 software (34). The ‘noeassign’ module of CYANA was used for the automated NOE cross-peak assignments. Input files for the program runs consisted of [¹H-¹H] chemical-shift lists, manually picked NOE cross-peaks and their intensities (from the H₂O NOESY spectra), and dihedral angle constraints derived from the ³J_{HNHα}-couplings (35). Default parameters were used in all calculations. The final cycle in each run was set to yield an ensemble consisting of 20 energy-minimized structures. The final assignment of the NOE cross-peaks was checked for its consistency with the manually assigned cross peaks.

Immunization of Rabbits with V3 Peptides

Twelve week old female, New Zealand white rabbits were purchased from the animal breeding center in the Weizamnn Institute of Science (Rehovot, Israel). Animals were treated according to the guidelines and under the supervision of the Animal Care and Use Committee. All the work was done under the supervision of the veterinary resources department. Animals were immunized 5 times at weeks 1, 4, 8, 13 and 38 with 250 μg of HPLC purified peptide. Peptide was mixed at 1:1 volume ratio to 1 ml with Complete Freund's Adjuvant (CFA) in the first injection and with Incomplete Freund's Adjuvant (IFA) in the second injection. Final boosts were given in PBS. Animals were bled 14 days after the 3^{rd} 4^{th} and 5^{th} immunization and at week 24.

ELISA for Peptide Binding

To test the binding of the resulting antibodies to the immunizing peptide, Reacti-Bind™ Streptavidin High Binding Capacity Coated Plates clear, 96-wells were used (PIERCE Cat No 15500). Plates were washed three times with phosphate buffer saline (PBS) with 0.1% BSA and 0.05% Tween-20 (wash buffer). 100 μl of the V3 or C4 biotinylated peptide (Table 4, herein below) at 1 μg/ml in wash buffer were added to each well and incubated for 2 hours with shaking at room temperature. After rinsing the ELISA wells, serial dilutions of the serum in wash buffer were added to each well and incubated for 1.5 hours at room temperature. This was followed by several washes and 45 minutes incubation with 1:2500 dilutions of secondary antibodies in wash buffer (HRP-conjugated donkey anti-rabbit-Jackson 711-035-152). The plates were washed and HRP substrate (TMB/E by Chemicon International) was added at RT. The reaction was stopped by adding 100 μl of 0.1% sodium fluoride and read at 650 nm in VersaMax microplate reader. In order to determine half-max binding values, 0.D values were plotted against the serum dilution and fitted using Origin software to one-site binding model. Data is presented as the reciprocal of the serum dilution at half maximum binding.

ELISA for Antibody Binding to gp120

To determine the level of serum-antibodies reactivity to gp120 by ELISA, JR-FL gp120 was expressed in HEK293 mammalian cells and subsequently purified. This gp120 molecule is truncated at both the N- and C-terminus and is termed ⁸⁸⁻⁴⁹²gp120. The segments coding for the V1 and V2 variable loops were deleted and replaced by a segment coding for gly-ala-gly (GAG). Moreover, two glycosylation sites were modified (N301Q and T388A). This gp120 construct (⁸⁸⁻⁴⁹²gp120ΔV1/V2, N301Q, T388A; SEQ ID NO: 16) was expressed in a mutated HEK293 cell line lacking the gene for N-acetylglucosaminyltransferase I. The expressed proteins are homogenously glycosylated with Man₅GlcNAc₂ glycans at sites normally occupied by complex or hybrid glycans. The protein was coated onto HisGrab™ Nickel Coated, High Binding Capacity Plates clear, 96-well (PIERCE Cat No 15142) for 2 hours with shaking at RT with 100 μl of ΔV1/V2 gp120 at 10 μg/ml in PBS. The following steps are as described above for peptide ELISA.

Neutralization Assay

Pseudoviruses single round of infections based neutralization assay was carried out by Monogram Biosciences, Inc. South San Francisco as previously described (37). Virus particles containing virus envelope proteins were produced by co-transfecting HEK293 cells with a plasmid expressing HIV-1 primary isolates Env plus an HIV genomic vector that contains the luciferase indicator gene. Murine leukemia virus (MLV) Env plasmid was used as negative control to assess non-specific neutralization. Recombinant viruses pseudotyped were harvested 48 hours post-transfection and incubated for 1 hour at 37° C. with serial two-fold dilutions of heat-inactivated rabbit sera starting at 1:10. U87 cells that express CD4 plus the CCR5 and CXCR4 co-receptors were inoculated with virus-serum dilutions. Virus infectivity was determined 72 hours post-inoculation by measuring the amount of luciferase activity expressed in infected cells. Neutralizing activity is displayed as the percent inhibition of viral replication (luciferase activity) at each antibody dilution compared with an antibody-negative control: % inhibition={1−[luciferase_{+Ab}luciferase_{-Ab}]}*100. Titers were calculated as the reciprocal of the plasma dilution conferring 50% inhibition (IC50).

Statistical Analysis

In the present neutralization assay, sera from 3 groups (P1-P3), each immunized with different V3 peptide, were tested against 7 different HIV strains. Each group consists of 4 rabbits (total of 12 sera). In order to asses the statistical significant of the neutralization data it was analyzed by multivariate analysis of variance, MANOVA. First, a vector of mean is calculated for each immunogen, which describes the mean response of each group to each of the 7 HIV strains. The purpose of MANOVA is to test whether these vectors of the different groups are sampled from the same sampling distribution. No interaction was found between immunogen and virus, justifying the multiple variant approach. Comparisons between pairs of immunogens were performed by contrast t-tests. For the analysis, the natural logarithm (ln) of the data was used; <10 values were calculated as 0.5 ln 10 (P<0.05 was considered significant).

Results

NOE Interactions in the Constrained Peptides

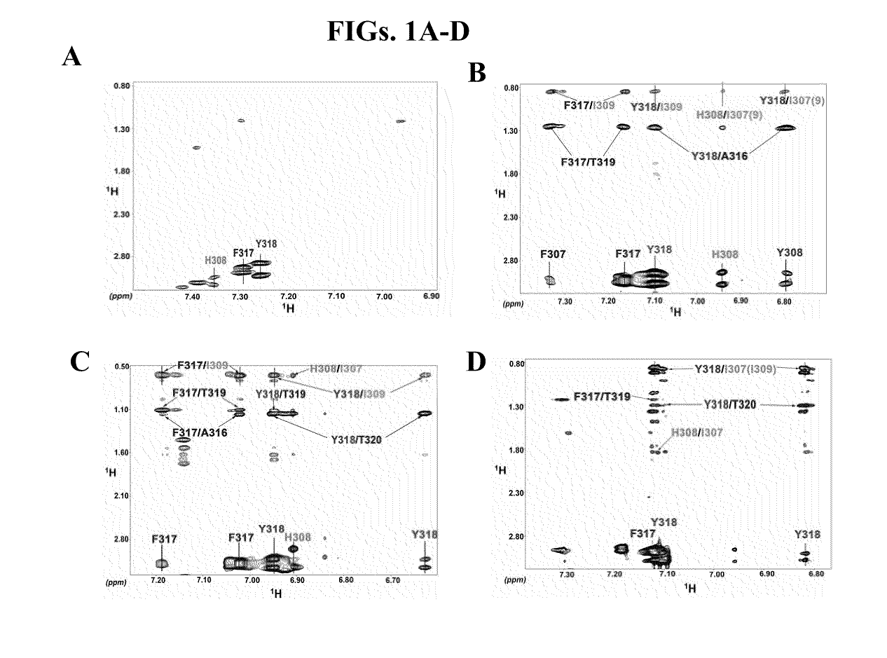

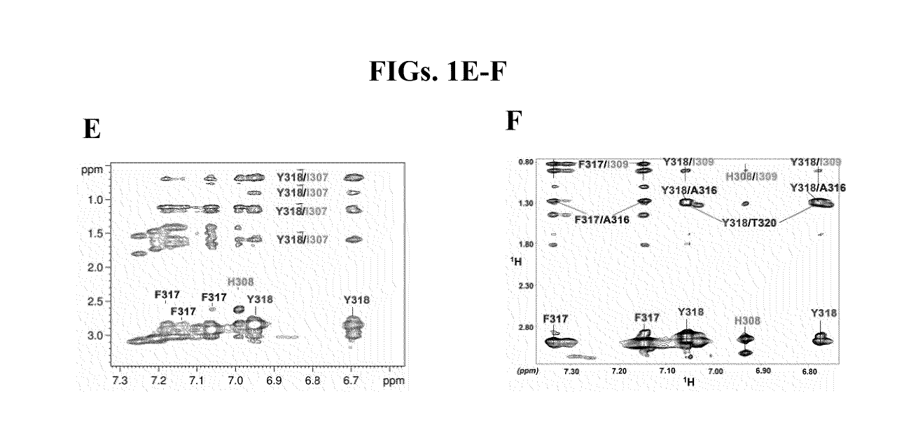

In order to explore the possibility of constraining the V3 conformation, a single disulfide bond was used. As a template, the consensus sequence of Glade-B virus that contains the entire epitope recognized by the antibody 447-52D was chosen. As mentioned above, the disulfide bond can dictate the pairing of the residue in the β-hairpin and also has the potential to dictate the register of the hydrogen bond forming residues. To assess the influence of the disulfide bond on the V3 conformation, the NOESY spectrum of a linear V3 peptide was measured. As shown in FIG. 1A, the linear V3_{JR-FL} peptide did not exhibit any long-range NOE interactions that are characteristic of a β-hairpin conformation.

To investigate how the location of the disulfide bond influences the conformation of the V3, the NOESY spectra of V3_{T303C,I323C}—SEQ ID NO: 13, V3_{T303C,E322C}—SEQ ID NO: 12, V3_{R304C,G321C}—SEQ ID NO: 11, V3_{K305C,T320C}-SEQ ID NO: 10 and V3_{I307C,T319C}—SEQ ID NO: 9 in which the location of the disulfide bond was changed systematically (Table 1). As indicated in FIGS. 1B and 1C, side chain protons within peptides V3_{T303C,I323C}—SEQ ID NO: 13 and V3_{T303C,E322C}—SEQ ID NO: 12 exhibited several i,i+2 NOE interaction (F317/T319, A316/Y318 in FIG. 1B and F317/T319, Y318/T320 in FIG. 1C), characteristic of a β-hairpin conformation. In addition, long-range NOE interactions were observed among the side chains of I307 and/or I309 (the chemical shift of the methyl protons of I307 and I309 overlaps) and the aromatic protons of residues F317 and Y318 at the C-terminal strand. However, these latter interactions indicate the formation of a cluster of hydrophobic side chains rather than a conformation similar to a β-hairpin, because in a β-hairpin F317 and Y318 should not point to the same direction. Another possibility is an equilibrium between two conformations or more, in one of them F317 opposes I309 while in another Y318 opposes I307.

When the position of the N-terminal cysteine was moved one residue inward from 303 to 304, the NOESY spectrum (FIG. 1D) reveals the appearance of pair-wise interactions characteristic of a β-hairpin (F317/T319, Y318/T320). The peptide V3_{R304C,G321C}—SEQ ID NO: 11—exhibited strong long range NOE interactions between the aromatic protons of Y318 and the methyl protons of I307 and/or I309 as shown in FIG. 1D. Thus, unlike the previous two peptides V3_{T303C, E322C}-1—SEQ ID NO: 12 and V3_{T303C,I323C}—SEQ ID NO: 13, the peptide V3_{R304C,G321C}—SEQ ID NO: 11 does not form a cluster of hydrophobic side chains. This conclusion is further supported by the observation of interactions between the aromatic protons of Y318 and the methyl protons of T320 indicating that the side chains of these two residues point approximately to the same direction. The NOE interactions between the aromatic protons of F317 and the methyl protons of T319 indicate that the side chains of these two residues are on the same face of the hairpin, which is different from the surface created by the side chains of I307, Y318 and T320.

The NOESY spectrum of V3_{K305C,T320C}—SEQ ID NO: 10—exhibited strong NOE interactions between many side chain protons of I307 and I309 with the aromatic side chain of F317 (FIG. 1E). The aromatic F317 ring interacts as well with side chain protons of R304, 5306 and H308. In addition, I307 also interact with the aromatic side chain of Y318. Several i,i+2 NOE interactions between side chain protons were detected between R315/F317, F317/T319, Y318/C320. An interaction between the aromatic protons of F317 and Y318 is also observed (data not shown). Although the appearance of multiple i,i+2 NOEs could support a β-hairpin, the overall data indicates the formation of a hydrophobic core with F317 interacting with numerous N-terminal side chains.

The NOESY spectrum of V3_{I307C,T319C}—SEQ ID NO: 9—exhibited strong NOE interactions between F317 with both methyl groups of I309 (FIG. 1F). The dispersion of the aromatic protons of this peptide allowed detection of NOE interaction between the aromatic protons of Y318 and H308 (data not shown). Only very weak interactions were observed between the γ2 methyl protons of I309 and Y318. Several i,i+2 NOE interactions between side chain protons were detected (A316/Y318, Y318/T320). Therefore, it can be concluded that the pattern of medium and long-range NOE interactions observed for V3_{I307C,T319C}—SEQ ID NO: 9—is characteristic of a β-hairpin conformation (although definition of ideal β-hairpin conformation requires the observation of additional long-range backbone-backbone interactions). The presence of cross peak between the adjacent A316/F317 corresponds to the presence of a five residue GPGRA turn, as a result of which these side chains would be in proximity.

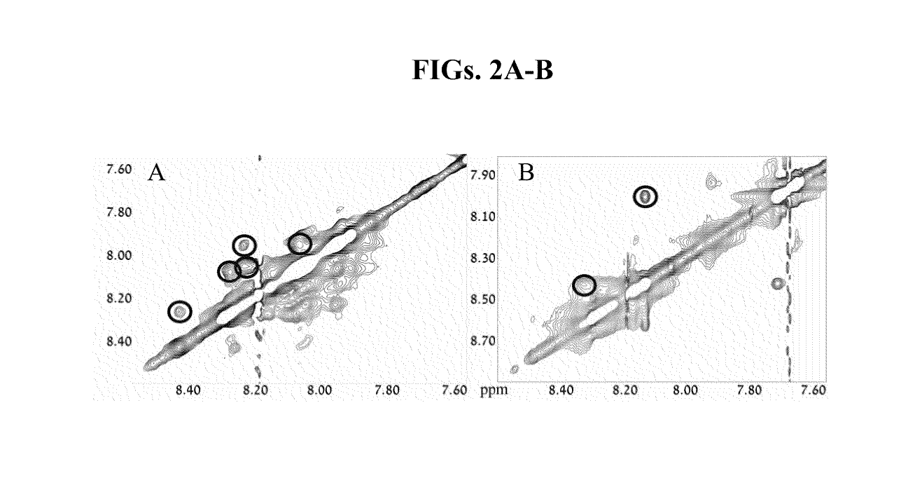

Multiple HN—HN interactions between adjacent residues (NN_{i,i+1}) interactions accompanied by strong αN_{i,i+1} are indicative of disordered conformation while absence or very weak NN_{i,i+1} interactions in the presence of strong αN_{1,i+1} indicate a more extended strand conformation. The peptide V3_{K305C,T320C}—SEQ ID NO: 10—reveals 12 medium-weak NN_{i,i+1} interactions, the peptide V3_{T303C,E322C}—SEQ ID NO: 12—revealed at least 8 medium-weak NN_{i,i+1} interactions, the peptide V3_{R304C,G321C}—SEQ ID NO: 11—exhibited 7 NN_{i,i+1} interactions considerably weaker than those observed for V3_{T303C, E322C}—SEQ ID NO: 12 (data not shown). The peptide V3_{T303C,E323C}—SEQ ID NO: 13—contains around 4 NN_{i,i+1} interactions (FIG. 2A). This data excludes the presence of extended conformation in those four peptides. On the other hand, only 2 NN_{i,i+1} interactions were observed for the peptide V3_{I307C,T319C}—SEQ ID NO: 9—as shown in FIG. 2B indicating that this peptide shows higher tendency to adopt an extended conformation resembling a β-strand conformation.

Structure of Peptide Constrained by Single Disulfide Bonds

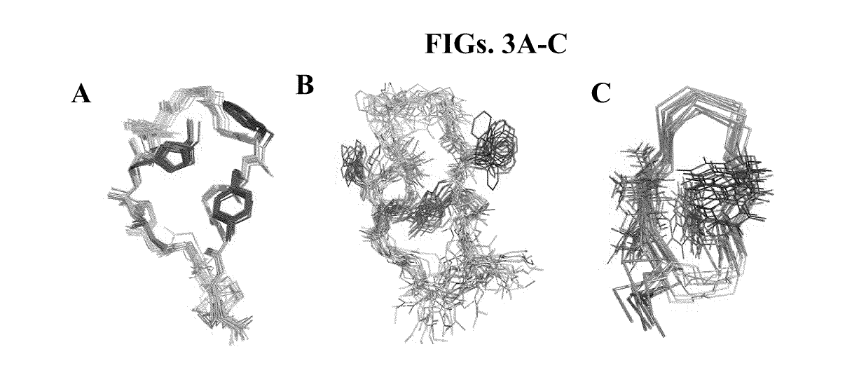

NMR data was used to determine the structure of three of the constrained peptides, namely V3_{T303C,E322C}—SEQ ID NO: 12, V3_{R304C,G321C}—SEQ ID NO: 11 and V3_{I307C,T319C}—SEQ ID NO: 9. The HOHAHA spectra indicate that conformational heterogeneity exists for all three peptides as judged by the appearance of multiple spin systems for some of the peptide residues. In all cases, a dominant spin-system from the major conformation could be identified. Severe cross-peak overlap and the appearance of multiple spin systems in all peptides' spectra complicated the sequential assignment procedure. Nevertheless, the present inventors have obtained 96.6%, 91.4% and 94.5% protons resonance assignment for V3_{T303C,E322C}—SEQ ID NO: 12, V3_{R304,G321C}—SEQ ID NO: 11 and V3_{I307C,T319C}—SEQ ID NO: 9, respectively. The structures of V3_{T303C,E322C}—SEQ ID NO: 12, V3_{R304C,G321C}—SEQ ID NO: 11 and V3_{I307C,T319C}—SEQ ID NO: 9, were determined on the basis of 269, 191 and 227 NMR-derived constraints, respectively. Superposition of the lowest energy structures of these three peptides are shown in FIGS. 3A-C. Although the V3_{T303C,E322C} (SEQ E ID NO: 12), and V3_{R304C, G321C} (SEQ ID NO: 11) peptides exhibited many interactions between side-chains and backbone atoms of residues on opposing strands (66 and 33 cross strands interactions for V3_{T303C,E322C} (SEQ ID NO: 12) and V3_{R304C,G321C} (SEQ ID NO: 11), respectively), neither of these peptides was found to adopt a β-hairpin conformation. The average backbone RMSDs (for all residues between, and included, the two cysteine residues in each peptide) for the 20 lowest energy structures, to the mean coordinates of the output bundles, are 0.64±0.28 Å for V3_{T303C, E322C} (SEQ ID NO: 12), 1.72±0.50 Å for V3_{R304C, G321C} (SEQ ID NO: 11) and 0.51±0.17 Å for V3_{I307C,T319C} (SEQ ID NO: 9).

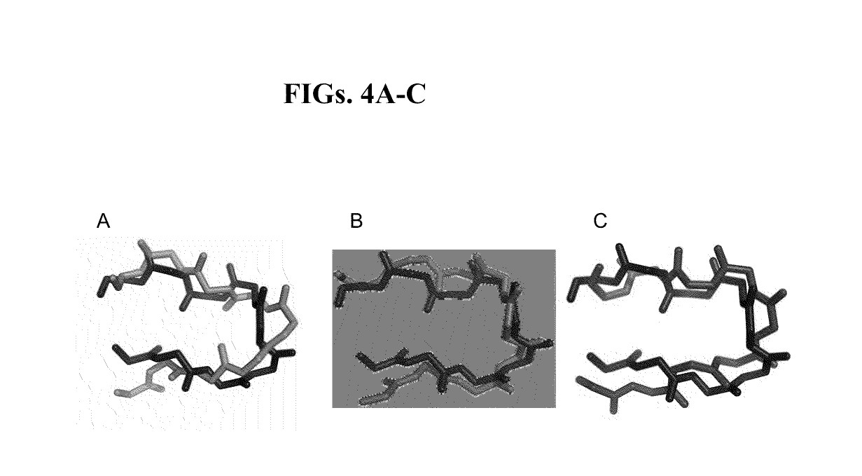

As shown in FIG. 3A, the side chains of residues H308, I309, F317 and Y318 of V3_{T303C,E322C} (SEQ ID NO: 12) point in the same direction resulting in the formation of a hydrophobic core. Examination of the deviation of the Ha chemical shifts from their random coil values reveals that only one residue is shifted by more than 0.1 ppm down-field, indicating the absence of any significant population in a β-strand conformation. The structure of V3_{R304C,G321C} (SEQ ID NO: 11) peptide (FIG. 3B) tends more towards a β-hairpin conformation, with the characteristic organization of alternative side chains of the N- and C-terminal strands. Thus, a distinct pairing of residues is observed and side chains of every other residue point in the same direction creating one surface composed of the side-chains of I307, I309 and Y318 and the opposite face formed by the side chains of H308 and F317. Residues K305, 5306, F317 and T319 of V3_{R304C,G321C} (SEQ ID NO: 11) exhibit chemical shifts higher by 0.1 ppm than random coil values indicating some population in extended conformation. The relative fewer NMR derived constraints used to calculate this peptide structure (191) is in accordance with the poorer convergence of the bundle of accepted structures. Surprisingly, residues R304, C305, 5306, G310, A316, F317, Y318, T319 and G321 of V3_{K305C,T320C} (SEQ ID NO: 10) exhibit chemical shifts higher by 0.1 ppm than random coil values, indicating some population in extended conformation. The structure of this peptide was not solved yet, however as indicated previously the observed NOE interaction implies the formation of a hydrophobic core rather that a β-hairpin. The structure of V3_{I307C,T319C}—SEQ ID NO: 9 (FIG. 3C) reveals characteristic features typical to a β-hairpin conformation. The side chains of residues I309 and F317 point in one direction, where those of H308 and Y318 point in the other direction of the β-hairpin. Superposition of the ordered region (H308-Y318) within either V3_{T303C,E322C} (SEQ ID NO: 12), V3_{R304C,G321C} (SEQ ID NO: 11) and V3_{I307C,T319C} (SEQ ID NO: 10) structures with V3_{JRFL} when bound to 447-52D antibody revealed a striking similarity between the backbone atoms in the aligned segments, with an RMSD of 2.08 Å, 1.48 Å and 2.02 Å, respectively (FIGS. 4A-C).

The Design of the Peptide Immunogens

Cysteine residues have low propensity to form hydrogen bonds with β-hairpin while the residues flanking the cysteine have high propensity. Therefore, in order to mimic the hydrogen bond network formed by residues 5306 and H308 in the N-terminal of V3 peptides bound to 447-52D, cysteines can only replace residues K305, I307 and I309. Unfortunately, these three residues form extensive interactions with 447-52D antibody in all the studied complexes. Among the three, I307 and I309 seem to be the most dominant in antibody peptide interactions (16, 17). A previous attempt by Varadarajan and his co-workers to constrain the V3 conformation by replacing I307 with cysteine resulted in an immunogen that did not elicit HIV-1 neutralizing antibody (21). The present inventors therefore decided to examine peptides with cysteine residues at positions 303 and 305. The replacement of T303 with cysteine leaves the entire sequence of the V3 epitope recognized by 447-52D intact. The replacement of K305 with cysteine involves the N-terminal residue of the 447-52D V3 epitope which forms extensive interactions with the antibody.

The present designed peptide immunogens are based on the V3_{JR-FL} sequence which is the consensus sequence for Glade-B R5 viruses and includes the entire epitope recognized by the 447-52D antibody (K305-T320). These peptides include seven additional residues at the N-terminus (²⁹⁸RPNNNTR³⁰⁴—SEQ ID NO: 14), like the peptides used by Haynes and coworkers (9). However, the V3 peptide immunogens are elongated by two additional residues at the C-terminus, i.e. E³²² and ^{I323}. E322 was added since an electrostatic interaction with R³⁰⁴ could further stabilize the β-hairpin conformation (38). I323 was added to reduce termini effect on the C-terminus of the V3 epitope. The V3 sequence is preceded by a T-helper epitope from the fourth constant region (C4) sequence of gp120_{JRFL}, i.e. ⁴²¹KQIIMNWQEVGKAMYA⁴³⁶—SEQ ID NO: 15, following the approach of Haynes and his co-workers (9). The peptide composed of C4 with a linear V3 is named hereafter P1 (SEQ ID NO: 1). The two other C4-V3 peptides are constrained by a disulfide bond in the V3 segment. C4-V3_{T303C-I323C} is named P2 (SEQ ID NO: 2) and C4-V3_{K3050-T320C} is named P3 (SEQ ID NO: 3) (Table 3). The peptide C4-V3_{T303C-I323C} (SEQ ID NO: 2) is constrained by a disulfide bond in a location that is completely outside the 447-52D epitope and enables the electrostatic interactions between R304 and E322.

Antibody Response Against the V3 Peptides

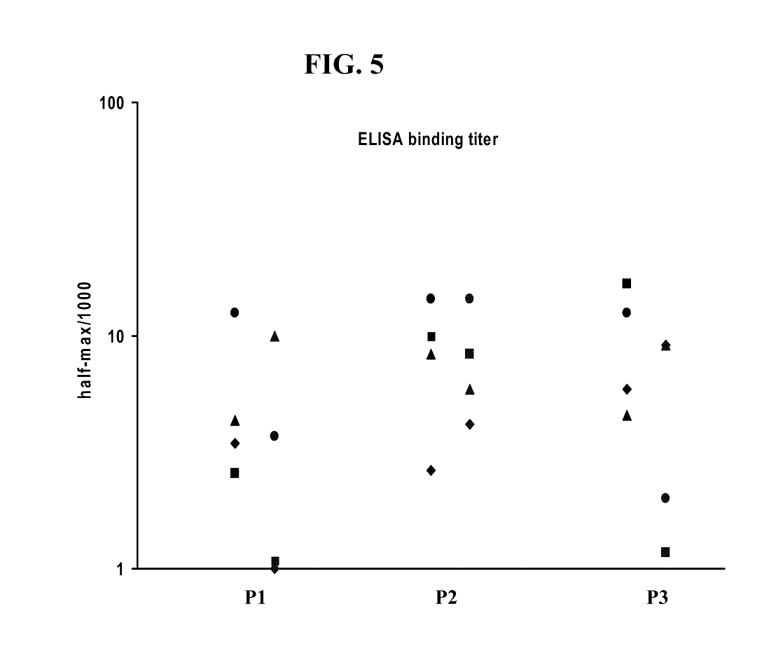

To test the reactivity of the serum to the immunizing peptide, each pre-immune sera and sera after the third immunization (post3) was tested in ELISA with the homologous V3 peptide. Endpoint binding titer and half-max are presented in Table 4 herein below and FIG. 5. Endpoint binding titer was set as the serial dilution with signal two times higher then the pre-immune sera.

TABLE 4
Summary of antibody binding for post3 serum against V3 peptide,
gp120 and C4 peptides.

| | | | gp120 | | | |
|---|---|---|---|---|---|---|
| | V3 Ab titer | | Ab titer | | C4 Ab titer | |
| | | Endpoint | | Endpoint | Half- | Endpoint |
| | Half-max | titer | Half-max | titer | max | titer |
| P1A | 3448 | 128,000 | 1000 | 32000 | 416 | >10000 |
| P1B | 2564 | 32000 | 1086.96 | 32000 | 147 | 2500 |
| P1C | 4347 | 128000 | 10000 | 512,000 | ND | ND |
| P1D | 12500 | 512,000 | 3703.7 | 128000 | ND | ND |
| P2A | 2631 | 128,000 | 4166 | 128,000 | 257 | 10000 |
| P2B | 10000 | 512,000 | 8333.33 | 512,000 | 456 | >10000 |
| P2C | 8333 | 128,000 | 5881.353* | 128,000 | ND | ND |
| P2D | 14285 | 512,000 | 14285.71* | 512,000 | ND | ND |
| P3A | 5882 | 128,000 | 9090.909 | 128,000 | 1428 | >10000 |
| P3B | 16666 | 512,000 | 1176.471 | 32000 | 218 | 10000 |
| P3C | 4545 | 128,000 | 9090.909 | 512,000 | ND | ND |
| P3D | 12500 | 512,000 | 2000 | 32000 | ND | ND |

Half-max values were determined by plotting O.D values against serum dilution, fitting to one-site binding model using Origin software and finding the serum dilution at half maximum
O.D. Endpoint titers are the points in which post immune ELISA signal is at least 2 times greater then for the pre immune sera.
ND—not done.
*1:100 dilutions was excluded from the calculation

High antibody titer of post3 sera is evident (FIGS. 5 and 6A-L). Endpoint titer are >10⁵ for all serums except for P1B. The binding titer for all sera is within the same order of magnitude. It should be stated that the binding titer for P1 group seems somewhat lower compared to the two other groups; this may imply that constrained peptides are better immunogens as they are more resistant to proteolytic degradation. Due to the large variability within each group this difference cannot be considered significant.

Cross-Reactivity of the Anti Peptide Antibody with gp120

To test the binding of the polyclonal serum to the native V3 loop, the present inventors tested the reactivity to a recombinant gp120 of the JR-FL strain, as described in material and methods. For each rabbit, pre-immune and post3 sera were tested for reactivity with the ΔV 1/V2 gp120. Half-maximum binding and endpoint binding titer is shown (Table 4, herein above, gp120 Ab titer). The data shows that all animals immunized with P2 had high binding to gp120 (end point>10⁵, half-maximum>4000) while for the two other groups the binding was variable and generally lower; only 2 serums in P1 and P3 groups had gp120 antibody titers comparable to that of the group immunized with P2 (Table 4, herein above).

Notably, reactivity with gp120 is highly correlated with peptide antibody titer in sera immunized with P2; With P2A<P2C<P2B<P2D for both V3 and gp120. Such correlation is not observed for the other immunogens. This is highly evident for the P3 immunogen where high levels of peptide antibody are not correlated with gp120 binding (FIGS. 2A-B and 3A-C). This suggests that the P2 immunogen presents the native V3 epitope in a form that is a better mimic of the native V3 conformation, in comparison with the linear peptide P1 and the constrained peptide P3.

Reactivity of the Anti-Sera with the C4 Helper Epitope

Since the present immunogens consist of two epitope, V3 and C4, it is of interest to test the reactivity of the post-immune serum to the C4 epitope. For that purpose, binding of the serum to biotynylated C4 peptide was determined by ELISA. Half-max and endpoint binding titers were calculated. The data indicated that the binding titer directed against the C4 epitope is about one order of magnitude lower than for the V3 epitope (Table 4, herein above). It is important to note that previous studies have shown that C4 peptide dose not elicit antibodies that can bind to gp120 (39, 40). Therefore, although relatively high antibody level was generated against C4, these are probably not relevant for gp120 binding and HIV neutralization.

Neutralization of HIV-1 Primary Isolates

To asses the immunogenic capabilities of the different immunogens of the present invention, post3 immune sera were tested for neutralization against a panel of 7 HIV-1 Glade-B viral strains (Table 5, herein below).

TABLE 5

| | |
|---|---|
| NL-43 | X4 |
| MN | X4 |
| SF-162 | R5 |
| NSC | R5 |
| Bal | R5 |
| JR-CSF | R5 |
| BX08 | R5 |

HIV-1 strains known to be sensitive to neutralization were used (with the exception of JR-CSF which is more resistant to neutralization). NSC is a neutralization sensitive R5 primary isolate from an acute infection (4l). Neutralization activity of the different sera is shown in Table 6, herein below.

TABLE 6
Summary of the IC50 serum dilution.

| Rabbit | | | | | | | | |
|---|---|---|---|---|---|---|---|---|
| sera | BaL | BX08 | MN | NSC | SF162 | JRCSF | NL43 | MLV |
| P1A | <10 | <10 | <10 | 53 | 53 | <10 | <10 | <10 |
| P1B | <10 | <10 | <10 | 11 | 63 | <10 | <10 | <10 |
| P1C | 12 | 60 | 244 | 32 | 15 | <10 | 19 | <10 |
| P1D | 15 | 31 | 38 | 62 | 964 | <10 | <10 | <10 |
| P2A | 12 | 27 | 30 | 1647 | 557 | <10 | <10 | <10 |
| P2B | 111 | 285 | 1178 | 350 | >5120 | 22 | 10 | <10 |
| P2C | 21 | 52 | 115 | 276 | 1150 | <10 | 42 | <10 |
| P2D | 16 | 55 | 133 | 190 | 1900 | <10 | <10 | <10 |
| P3A | <10 | 64 | <10 | 13 | 111 | <10 | <10 | <10 |
| P3B | <10 | <10 | <10 | <10 | <10 | <10 | <10 | <10 |
| P3C | <10 | 27 | 15 | 165 | 191 | <10 | 32 | <10 |
| P3D | <10 | <10 | <10 | <10 | 14 | <10 | <10 | <10 |

All sera were diluted X2 starting at 1:10; Titers, calculated as the reciprocal of the plasma dilution conferring 50% inhibition (IC50) are presented. MLV negative control is also shown.

Clear differences can be seen between the groups: all P2 sera neutralize Bal, BX08, MN, NSC, SF162 strains with much better titers than the groups immunized with P1 or P3. When subjected to statistical analysis, the differences between P2 vs. P1 and P2 vs. P3 was significant (P=0.0142 and 0.0035, respectively), while the difference between P1 and P3 was not (P=0.3951). This data clearly demonstrate that the constrained peptide P2 is a better immunogen compared to the linear peptide P1 or the other constrained peptide P3. NL-43, which is an X4 lab adapted strain, was not neutralized. The reason for that is not clear but can result from its unusual sequence. JR-CSF is more difficult to neutralize and was generally not neutralized; nevertheless, the serum indicated P2B shows some neutralization of that strain. This serum had one of the highest binding titer to the immunizing peptide and to gp120 as well as the highest IC-50 neutralizing titer for other strains.

Discussion

The NMR analysis of the linear and constrained V3 peptides indicates that some of the constrained peptides reveal conformation that resembles a β-hairpin in terms of side-chain interactions. However, the absence of backbone-backbone interactions and the small deviations of the Ha chemical shifts from random coil values indicate that a characteristic β-hairpin conformation could not be obtained using a disulfide bond. The location of the disulfide bond influences the rigidity of the V3 conformation and its ability to form a β-hairpin conformation. The closer the location of the disulfide bond to the GPGR segment is, the better the V3 structure resemblance to a β-hairpin conformation. Mimicking the R5 conformation of the V3 requires replacement of one of the residues at positions 303, 305, 307 or 309 with a cysteine. The drawback is that the side-chains of residues K305, I307 and I309 form extensive interaction with the 447-52D antibody and their replacement may abolish binding. A failure to obtain HIV-1 neutralization after immunizing with a V3 containing recombinant protein in which I307 was replaced by cysteine already indicated a potential problem with replacement of residues involved in 447-52D interactions (2l). Therefore the present inventors tested two constrained peptides in which the disulfide bond is located further away from the GPGR segment. In P3, K305 was replaced by a cysteine and in P2, T303 was replaced by a cysteine.

HIV-1 neutralization tested with the sera obtained after immunization with the linear peptide P1 (SEQ ID NO: 1) and with P2 (SEQ ID NO: 2) and P3 (SEQ ID NO: 3) indicates that a disulfide bond between residues 303 and 323 results in a much better neutralization in comparison with a linear peptide and in comparison with a peptide constrained by a disulfide bond between residues 305 and 320. The present examples demonstrate for the first time that a constrained peptide elicits better cross-reactive and neutralizing antibodies than linear peptides. The present examples also demonstrate the importance of optimizing the location of the disulfide bond and have shown that a disulfide bond located in certain “wrong” positions can actually result in an immunogen that gives worse neutralization results in comparison to the linear peptide.

### Example 2

### Additional Constrained Peptides Useful as HIV Vaccines

Materials and Methods

Chain assembly and purification of the linear (SH) containing C4-V3 peptides. The C4-V3 peptides were synthesized on a 443A peptide synthesizer (Applied Biosystems) using 0.1 mM fast Fmoc chemistry. Approximately 160 mg (0.1 mM) of preloaded Fmoc-Ile Wang resin (0.6 mmol/g) or Fmoc-Cys(Trt) Wang resin (0.5 mmol/g) were used. The first 10 amino acids were attached using single coupling, the remaining amino acids were assembled into the peptide chain using double coupling. The unreacted amino group was capped with acetic anhydride at the end of each coupling step. The resin weight gain was about 90% of theoretical and peaks with expected m/z values were observed in the mass spectrum of the crude peptide. The peptides were cleaved from the resin using a water/TFA cocktail with appropriate scavengers. In a representative procedure, to a vial containing 220 mg of the resin was added a solution of water (0.50 mL), phenol (500 mg), 1,2-ethanedithiol (0.25 mL), thioanisole (0.50 mL), triisopropylsilane (0.10 mL) and trifluoroacetic acid (TFA; 10 mL) and the resulting mixture was stirred at 0° C. for 1 hour and at room temperature for 2 hours. Resin was separated from solution by filtration and washed two times with TFA. The combined filtrate was concentrated on a rotary evaporator under vacuum at a temperature below 30° C. and the resulting residue was treated with cold diethyl ether (40 mL). Precipitated peptide was isolated by centrifugation and ether was removed by decantation. After washing the solid precipitate was dried, dissolved in 2 mL acetonitrile (0.1% TFA), 4 mL of water (0.1% TFA) was added, the solution was frozen in dry ice and lyophilized for 48 hours to give final crude peptide in about 70% yield by weight.

For the synthesis of biotinylated homologs used in binding studies (Table 7), herein below, the resin was split into two parts (1:3) after completion of the assembly of the V3 epitope. A spacer sequence, mainly Gly-Ser-Gly, was built at the N-terminus of 25% of the peptide-resin followed by incorporation of biotin at the N-terminus. The couplings were carried out on the peptide synthesizer using the normal HOBT/HBTU activation method.

Preparative HPLC purification of linear peptide was carried out on a Waters DeltaPak column 19×300 mm using a 20-50% acetonitrile/water gradient in 80 min with a flow rate of 5 ml/min and detection at 220 nm and 280 nm. Both solvent reservoirs contained 0.1% TFA. Usually about 15 mg of crude peptide were dissolved in 1 ml of acetonitrile/water (1:4) and injected onto the column. Analytical HPLC of the linear peptides was carried out on a Zorbax Eclipse XDB-C8 column 4.6×150 mm using detection at 220 nm detection and 10-60% acetonitrile/water gradient over 20 min with a flowrate of 1 ml/min. The linear peptides used for cyclization were >90% homogeneous.

Disulfide bond formation. Peptides were cyclized using either ferricyanide mediated oxidation, DMSO mediated oxidation or glutathione mediated oxidation depending on the sequence and length of the peptide. In general the reaction progress was monitored by electron spray ionization mass spectrometry. In most of the cases the linear C4-V3 peptides were cyclized using oxidized glutathione (GSSG) as the oxidant. In a typical procedure, a solution of linear C4-V3 peptide (2.7 mg) dissolved in 3 mL of water containing 0.1% of TFA was added dropwise to a solution of GSSG (14 mg) in 80 mL of ammonium acetate (0.1 M, pH7.9). The combined solution was stirred overnight at room temperature. HPLC analysis showed almost no change in retention time and the progress of the reaction was monitored by MS analysis. After completion of the reaction, the solution was acidified to pH 2.0 by dropwise addition of 0.5 mL of TFA, filtered and the filtrate was loaded onto a preparative Waters C18 Delta Pak column. Product was eluted using a 10-50% acetonitrile/water gradient over 80 min. Both solvents contained 0.1% TFA. The pure fractions were combined and lyophilized to result in about 1.3 mg of pure cyclic peptide. The cyclic C4-V3 peptide was >95% homogeneous as analyzed by HPLC and the MS difference between linear and cyclic was 2 Da as expected.

Immunization of rabbits with V3 peptides. Twelve week old female New Zealand white rabbits were purchased from the animal breeding center at the Weizmann Institute of Science (Rehovot, Israel). Animals were treated according to the guidelines and under the supervision of the Animal Care and Use Committee. All the work was done under the supervision of the veterinary resources department. Animals were immunized up to five times at weeks 1, 4 8, 13 and 37 with 250 μg of HPLC purified peptide in phosphate buffered saline (PBS) or 250 μg gp120 in 50 mM Tris-HCl, 300 mM NaCl administrated intramuscularly. Peptide was mixed at 1:1 volume:volume ratio with Complete Freund's Adjuvant (CFA; 1 mL) at the first injection and Incomplete Freund's Adjuvant (IFA) in the second injection; further boosts were administered with no adjuvant. Animals were bled 10 days after each boost starting from the 3^{rd} immunization.

Expression and purification of gp120. The vector pSyn gp120 that encodes gp120_{JR-FL} was kindly provided by the NIH AIDS reagent program (www.aidsreagent.org). From this vector, a gene encoding residues 88-492 of gp120_{JR-FL} i.e. ⁸⁸⁻⁴⁹²gp120 was constructed and inserted into the pIRES vector which was developed for high level expression in HEK293 (Clontech, Mountain-View, Calif.). This vector encodes the IgK secretion signal, enabling secretion of the gp120 protein to the growth medium and a 6× histidine tag followed by a Tobacco Etch Virus (TEV) protease recognition site at the N-terminus of gp120. Additionally the segments coding for the V1 and V2 variable loops were deleted and replaced by a segment coding for Gly-Ala-Gly (GAG) and two glycosylation sites were modified (N301Q and T388A). These modifications have been reported to increase susceptibility to neutralization by CD4-binding site antibodies(43). This gp120 construct, ⁸⁸⁻⁴⁹²gp120ΔV1/V2, N301Q, T388A, (referred to as gp120) was stably transfected into a mutated HEK293 cell line lacking the gene for N-acetylglucosaminyltransferase 1(44). The expressed proteins are homogenously glycosylated with Man₅GlcNAc₂ glycans at sites normally occupied by complex or hybrid glycans. The protein was purified initially on a 50 ml Cibacron Blue Sepharose column (GE Healthcare), followed by a 5 ml HisTrap HP column purification (GE Healthcare). The eluted fraction was cleaved by TEV protease, followed by an additional purification on a Ni column to remove the TEV and uncleaved gp120. Finally the protein was purified on a superdex 200 16/60(GE Healthcare). The homogenously glycosylated 45 kD protein was identified by SDS Polyacrylamide Gel Electrophoresis and superdex 200 10/300 analytical gel filtration.

Determining peptide binding titers by ELISA. To test the binding of the resulting antibodies to the immunizing peptide, Reacti-Bind™ Streptavidin High Binding Capacity Coated Plates clear, 96-wells were used (PIERCE Cat No 15500). All procedures were done at room temperature. Plates were washed three times with PBS, 0.1% BSA, and 0.05% Tween-20 (wash buffer). 100 μl of the V3 or C4 biotinylated peptide (Table 1) at 1 μg/ml in wash buffer were added to each well and incubated for 2 hours with shaking. After rinsing the ELISA wells, serial dilutions of the serum in wash buffer were added to each well and incubated for 1.5 h. This was followed by several washes and 45 minutes incubation with 1:2500 dilutions of secondary antibodies in wash buffer (HRP-conjugated donkey anti-rabbit-Jackson 711-035-152). The plates were washed and HRP substrate (TMB/E by Chemicon International) was added. The reaction was stopped by adding 100 μl of 0.1% sodium fluoride and OD was read at 650 nm in a VersaMax microplate reader.

Alternatively Ni-column purified His-tagged gp120 (see above) was coated onto HisGrab™ Nickel Coated, High Binding Capacity Plates clear, 96-well (PIERCE Cat No 15142) for 2 hours with shaking with 100 μl of ⁸⁸⁻⁴⁹²gp120ΔV 1/V2 at 10 μg/ml in PBS. Subsequent steps are as described above for peptide ELISA.

In order to determine half-max binding values, OD values were plotted against the serum dilution and fitted using Origin software to a one-site binding model. Data is presented as the reciprocal of the serum dilution at half maximum binding.

When testing for binding to reduced V3 peptides, the peptides were incubated overnight in wash buffer supplemented with 10 mM DTT, followed by incubation on the plate with 10 mM DTT. Serum dilutions and washes were done in wash buffer supplemented with 2 mM DTT. Linear peptide was treated the same as the control.

Neutralization assay. Pseudoviruses single round of infection-based neutralization assay was carried out by Monogram Biosciences, Inc. South San Francisco as described herein above. Virus particles containing virus envelope proteins were produced by co-transfecting HEK293 cells with a plasmid expressing HIV-1 primary isolates Env plus an HIV genomic vector that contains the luciferase indicator gene. Murine leukemia virus (MLV) Env plasmid was used as a negative control to assess non-specific neutralization. Recombinant pseudotyped viruses were harvested 48 h post-transfection and incubated for 1 h at 37° C. with serial two-fold dilutions of heat-inactivated rabbit sera starting at 1:10. The virus/serum dilutions were incubated with U87 CD4⁺, CCR5⁺ and CXCR4⁺ cells. Virus infectivity was determined 72 h post-inoculation by measuring the amount of luciferase activity expressed in infected cells. Neutralizing activity is displayed as the percent inhibition of viral replication (luciferase activity) at each antibody dilution compared with no antibody sample, % inhibition={1−[luciferase_{+Ab}/luciferase_{-Ab}]}*100. Titers were calculated as the reciprocal of the serum dilution conferring 50% inhibition (IC50) or 90% inhibition (IC-90).

Statistical analysis. In order to assess the difference in binding to the immunizing V3 peptide and gp120 a one sample T-test for a hypothetical mean value of 1 was used. Unpaired T-test to compare two means was used to evaluate differences in binding to cyclic vs. reduced V3 peptide. Analysis was performed using the GraphPad QuickCalc Internet tools (http://wwwdotgraphpaddotcom/quickcalcs/). P-value<0.05 was considered significant.

### Results

Design and synthesis of disulfide-constrained V3-peptide immunogens. The constrained peptides used as immunogens in the present example were based on the consensus sequence for Glade-B R5 viruses and included the entire epitope recognized by the 447-52D antibody (K305-T320) (Table 7, herein below) with seven additional residues at the N-terminus (²⁹⁸RPNNNTR³⁰⁴—SEQ ID NO: 14) and 2 or 4 residues at the C-terminus (³²²EIIC³²⁵—SEQ ID NO: 29). In this example, the present inventors tested peptides constrained to either the R5A or the R5B conformation of the V3 using disulfide bonds involving residues 301, 303 or 305.

TABLE 7
Immmunogenic peptides

| | |
|---|---|
| C4-V3 linear | KQIINMWQEVGKAMYA- |
| | RPNNNTRKSIHIGPGRAFYTTGEI (SEQ ID NO: 1) |
| C4-V3 T303C-I323C (R5B) | KQIINMWQEVGKAMYA- |
| | RPNNNCRKSIHIGPGRAFYTTGEC (SEQ ID NO: 2) |
| C4-V3 K305C-T320C (R5A) | KQIINMWQEVGKAMYA- |
| | RPNNNTRCSIHIGPGRAFYTCGEI_(SEQ ID NO: 3) |
| C4-V3 K305C-G321C (R5B) | KQIINMWQEVGKAMYA- |
| | RPNNNTRCSIHIGPGRAFYTTCEI_(SEQ ID NO: 30) |
| C4-V3 T303C-E322C (R5A) | KQIINMWQEVGKAMYA- |
| | RPNNNCRKSIHIGPGRAFYTTGCG_(SEQ ID NO: 31) |
| C4-V3 N301C-G325C (R5B) | KQIINMWQEVGKAMYA- |
| | RPNCNTRKSIHIGPGRAFYTTGEIIC_(SEQ ID NO: 32) |

Peptides used for ELISA

| | |
|---|---|
| V3L | Biotin-GSGTRKSIHIGPGRAFYTTGEI (SEQ ID NO: 4) |
| V3 (T303C-I323C) | Biotin-GSGCRKSIHIGPGRAFYTTGEC (SEQ ID NO: 5) |
| V3 (K305C-T320C) | Biotin-GSGTRCSIHIGPGRAFYTCGEI (SEQ ID NO: 6) |
| C4 | Biotin-GSGKQIINMWQEVGKAMYA (SEQ ID NO: 7) |
| V3 (K305C-G321C) | Biotin-GSGTRCSIHIGPGRAFYTTCEI (SEQ ID NO: 40) |
| V3 (T303C-E322C) | Biotin-GSGCRKSIHIGPGRAFYTTGC (SEQ ID NO: 41) |
| V3 (N301C-G325C) | Biotin-GSGCNTRKSIHIGPGRAFYTTGEIIC |
| | (SEQ ID NO: 42) |

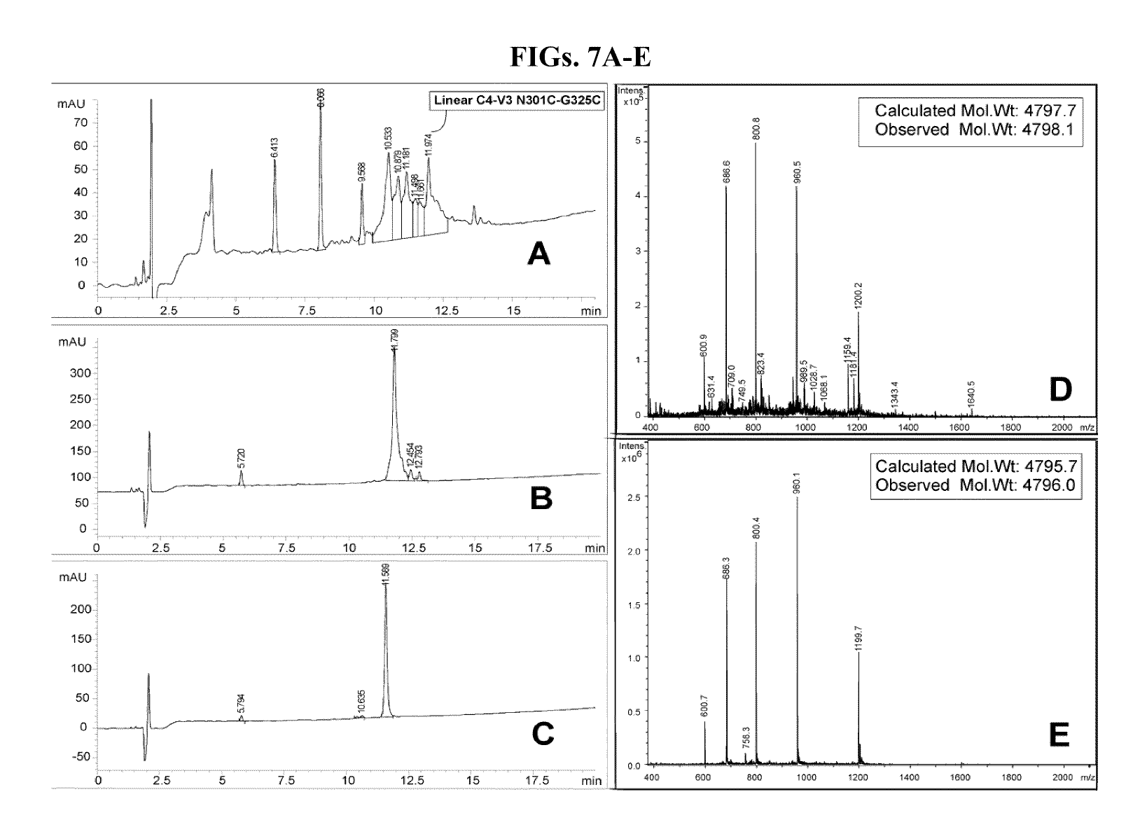

The immunogens used in this investigation contained 40 or 42 residues and regions with a significant tendency to assume β-sheet structures. β-Sheet formation during solid phase peptide synthesis often buries the chain ends and prevents chain elongation. Although all peptides were synthesized using automated solid phase peptide synthesizer with double coupling and capping after each step, the final crude product had significant heterogeneity (FIG. 7A). Moreover, in several syntheses early termination of the chain assembly was not successful. Truncation sequences formed in these syntheses were identified by mass spectrometry and the difficult couplings were circumvented by changing the coupling conditions or lowering the substitution on the resin to eliminate intermolecular interactions of growing peptide chains. Despite the synthetic challenges, using ESI MS the present inventors were able to identify the linear disulfhydryl precursor product and using preparative reversed phase HPLC, this was purified to >90% homogeneity (FIG. 7B). The linear sulfhydryl containing C4-V3 peptides or their biotinylated homologs were oxidized to form the constrained immunogens using a variety of procedures (See experimental procedures). No change in retention was discerned on reverse phase HPLC and the cyclization was monitored by mass spectrometry. In all cases, the final cyclic peptide was highly homogeneous (FIGS. 7C and 7E. Notice a 2 Da difference in mass between 7D and 7E). The degree of cyclization was also ascertained by slowly scanning the M/Z peaks and monitoring the isotope distribution (data not shown). The MS results enabled the distinguishing of small amounts of linear peptide in the cyclic product. All peptides used in binding or neutralization studies were >95% cyclic.

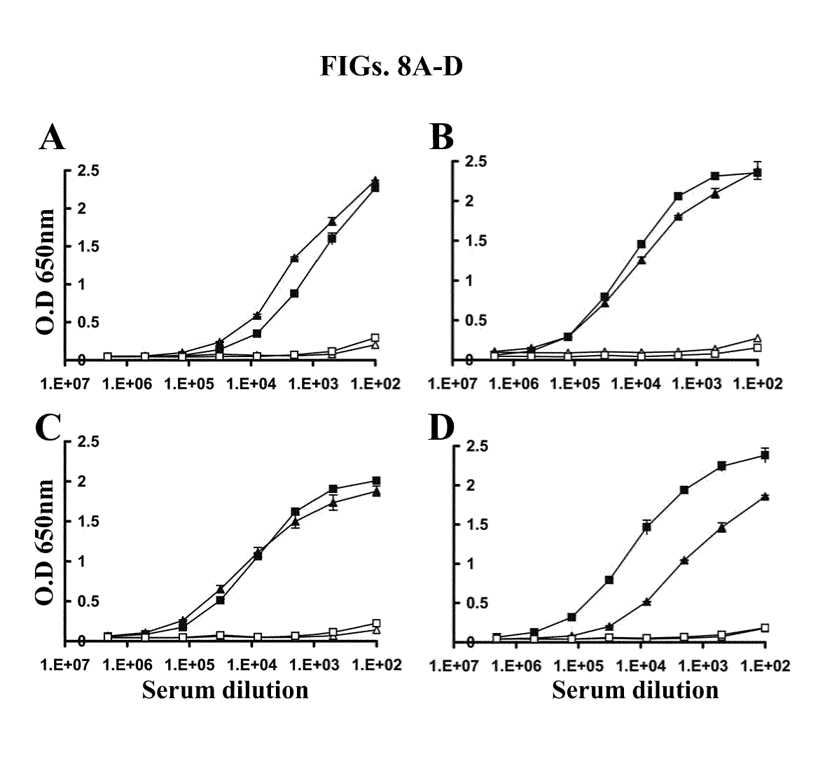

Reactivity of the C4-V3 immune sera with V3 and C4 peptides. A list of the constrained peptides used for immunization is presented in Table 7, herein above. For comparison a gp120 and a linear C4-V3 immunogen was also tested. Initially, several peptides were subjected to a 4^{th} and a 5^{th} boost at weeks 13 and 37, respectively. The post-4 sera displayed a small reduction in binding titer to the corresponding V3 immunogen compared to the post-3 sera. A test bleed done at week 24 in order to follow the V3 titer also indicated some reduction in antibody levels. The animals were rested and boosted for the fifth time at week 37. Serum drawn after the 5^{th} boost had lower levels of V3 antibodies than post-3 sera (data not shown). Therefore, sera drawn after the 3^{rd} boost were considered optimal, and tested for gp120 binding and neutralization of HIV-1 Glade B viral strains, and three boosts were adopted as the standard protocol for all further immunizations. High levels of V3 specific antibodies were observed after the third peptide boost for all C4-V3 immune sera, with geometric-mean-titer (GMT) values for half-maximal binding for the homologous V3 peptides from 3,038 to 19,947 (Table 8, herein below and FIGS. 8A-D).

TABLE 8

| Rabbit | Immunogen | V3 | gp120 | C4 |
|---|---|---|---|---|
| B712 | C4-V3L | 1786 | 658 | 416 |
| B707 | C4-V3L | 1786 | 840 | 147 |
| B702 | C4-V3L | 12500 | 7692 | ND |
| B722 | C4-V3L | 4167 | 2941 | ND |
| | GMT | 3590 | 1880 | |
| B717 | C4-V3K305C-T320C | 8333 | 8333 | 1428 |
| B715 | C4-V3K305C-T320C | 2381 | 1429 | 218 |
| B719 | C4-V3K305C-T320C | 11111 | 7692 | ND |
| B723 | C4-V3K305C-T320C | 4348 | 1961 | ND |
| | GMT | 5564 | 3661 | |
| B866 | C4-V3K305C-E322C | 4348 | 4000 | |
| B871 | C4-V3K305C-E322C | 1563 | 1563 | |
| B872 | C4-V3K305C-E322C | 2381 | 1493 | |
| B873 | C4-V3K305C-E322C | 5263 | 3333 | |
| | GMT | 3038 | 2362 | |
| B961 | C4-V3T303C-E322C | 20000 | 25000 | |
| B962 | C4-V3T303C-E322C | 10000 | 10000 | |
| B963 | C4-V3T303C-E322C | 14286 | 14286 | |
| B964 | C4-V3T303C-E322C | 50000 | 50000 | |
| | GMT | 19441 | 20557 | |
| B716 | C4-V3T303C-I323C | 2381 | 2778 | 257 |
| B720 | C4-V3T303C-I323C | 6250 | 7692 | 456 |
| B714 | C4-V3T303C-I323C | 5882 | 5000 | ND |
| B718 | C4-V3T303C-I323C | 12500 | 14286 | ND |
| | GMT | 5751 | 6250 | |
| B889 | C4-V3N301C-G325C | 50000 | 50000 | |
| B890 | C4-V3N301C-G325C | 11111 | 8333 | |
| B892 | C4-V3N301C-G325C | 14286 | 10000 | |
| | GMT | 19947 | 16091 | |
| B955 | gp120 | 5882 | 16667 | |
| B958 | gp120 | 3704 | 12500 | |
| B959 | gp120 | 2703 | 14286 | |
| B960 | gp120 | * | * | |
| | GMT | 3891 | 14384 | |

^{a} Binding of the C4-V3 and gp120 elicited immune sera to V3 peptides, gp120 and the C4 peptide. Immune-sera were obtained after three immunizations and half-maximal values of the binding reaction were determined. ND—not determined. Geometric mean titer (GMT) is presented.
* Undetectable levels of binding (excluded from calculation).

The highest titers were obtained for C4-V3_{N301C-G325C} (SEQ ID NO: 32) and C4-V3_{T303C-E322C} (SEQ ID NO: 31). To verify that most of the antibody response was directed against the V3 epitope, the reactivity of several of the sera was tested for V3 and C4 separately. A 4-16 fold lower GMT values for half maximum binding was obtained for C4 in comparison with V3 (Table 8, herein above).

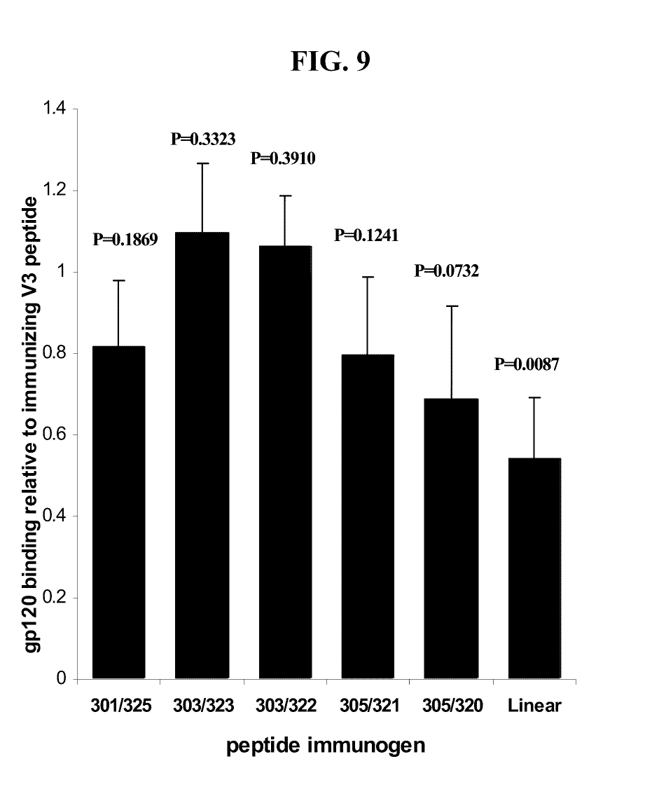

Cross-reactivity of the C4-V3 immune sera with gp120. A prerequisite for neutralization by a vaccine elicited sera is that it will be cross-reactive with gp120. Therefore, the pre-immune and the immune sera were tested for binding to the ΔV 1/V2 gp120 construct in which the V3 is fully exposed (FIG. 9, Table 8, herein above). For each serum the reduction in binding from the corresponding V3 peptide and gp120 was calculated and the data is presented as the average of the ratio between gp120 and the immunizing-peptide binding (FIG. 9). The two peptides constrained by a disulfide bond involving residue 303 elicited sera that bound gp120 as strongly as they bound the V3-peptide immunogen, while the sera from the rabbits immunized with the linear V3 (V3L) peptide was the least cross reactive to gp120 with an average ratio of 0.54 (p=0.0087) between gp120 and V3L binding (FIG. 9). The average gp120/peptide binding ratios of the serum induced by C4-V3_{K305C-T320C} (SEQ ID NO: 3) and C4-V3_{K305C-G321C} (SEQ ID NO: 30), which were constrained to the R5A and R5B conformation, respectively, were 0.69 and 0.80, respectively, while the sera elicited by C4-V3_{N301C-G325C} (SEQ ID NO: 32) had a gp120/constrained peptide immunogen binding ratio of 0.82. For these last three peptides the reduced binding to gp120 was less consistent than for the linear peptide as evidence by larger standard deviation which did not reach statistical significance as judged by the T-test (P value ranging from 0.07-0.19). Thus, although the differences in gp120 cross-reactivity among the different peptides' immune-sera are not large, it is clear that peptides constrained by a disulfide bond involving residue 303 exhibit higher cross-reactivity with gp120 in comparison with the sera elicited by the flexible linear peptide and the other constrained C4-V3 peptides.

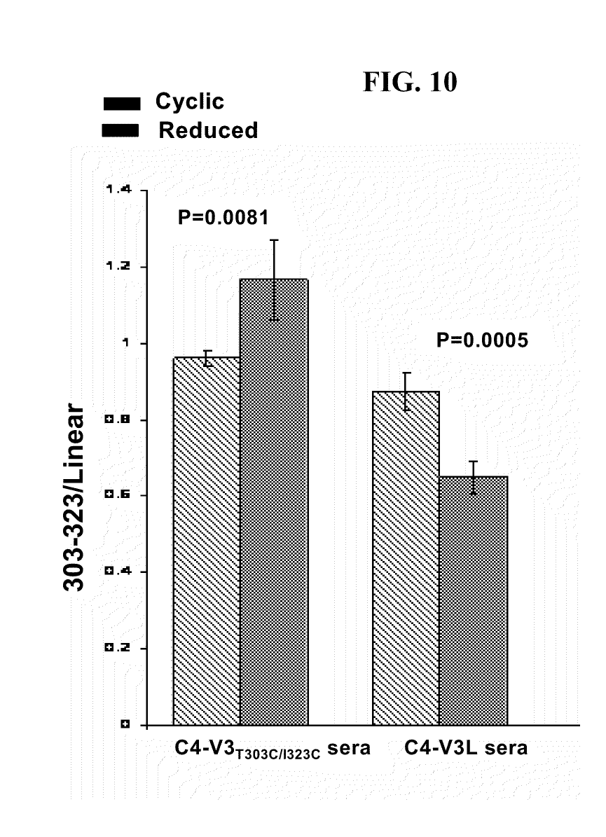

The reactivity of the peptide immune-sera is conformation dependent To further characterize the conformational specificity of the immune sera the binding of the C4-V3L (SEQ ID NO: 1) and C4-V3_{T303C-I323C} (SEQ ID NO: 2) immune-sera to the V3_{T303C;I323C} (SEQ ID NO: 2) peptide was tested in a reduced and cyclic form and as a control the binding to V3L in the presence and absence of reducing agent was tested (FIG. 10). For the C4-V3L immune sera, the ratio between cyclic V3_{T303C-I323C} binding and V3L binding was 0.65 and the ratio between reduced V3_{T303C-I323C} and V3L binding was 0.875, indicating that most of the decrease in the binding of the C4-V3L immune sera to oxidized V3_{T303C-I323C} compared with the V3L peptide is due to the disulfide-bond constraint (P=0.005 for reduced vs. cyclic). The ratio between C4-V3_{T303C-I323C} immune-sera binding to cyclic V3_{T303C-I323C} and V3L was 1.17 while the ratio between reduced C4-V3_{T303C-I323C} and V3L binding was 0.96. (P=0.008 for reduced vs cyclic). These results indicate that the C4-V3_{T303C-I323C} immune sera bind slightly better to the peptide constrained by a disulfide bond implying recognition of the conformation of this epitope.

Antibody response of gp120 based immunogen. The present inventors wished to compare the immune response against the C4-V3 peptide immunogens with that obtained against gp120 to determine whether there is any advantage in using constrained V3 peptides to obtain a potent HIV-1 neutralizing response. The gp120 construct used as immunogen in this study contained the full length V3 loop, lacked the first and second variable loops as well as the first 86 and last 19 N- and C-terminal residues, respectively, and is homogenously glycosylated with Man₅GlcNAc₂ glycans at sites normally occupied by complex or hybrid glycans (see experimental procedures). Three out of the four rabbits raised a strong anti-gp120 immune response; however one rabbit had undetectable antibody levels against both gp120 and V3 (B960) and therefore was excluded from the GMT calculation and from the analysis (Table 8, herein above). The GMT for half-maximal gp120 binding of the gp120 immune sera was 14,384, comparable with the reactivity with the homologous V3 immunogen of immune sera of constrained peptides that elicited strong antibody responses such as C4-V3_{N301C-G325C} (SEQ ID NO: 32) and C4-V3_{T303C-E322C} (SEQ ID NO: 31). The V3 directed antibody response of the gp120-immune sera, as measured by V3_{T303C-I323C} (SEQ ID NO: 2) binding, were 2.8-5.3 lower than that against gp120, with GMT for half-maximum binding of 3,891. The peptide V3_{T303C-I323C} was used in this experiment to assess the V3-directed response of the gp120 immune-sera because very good correlation between gp120 and peptide binding was observed for this peptide (see above). The results indicate that gp120 can elicit an antibody response to a number of epitopes, and a relatively high level of V3 directed antibody was obtained using the gp120 construct in which the V3 loop is fully exposed by deletion of the V1 and V2 loops and due to the glycosylation by short carbohydrate chains.

Neutralization of glade-B HIV-1 isolates by C4-V3 and gp120 immune sera. A pseudovirus based neutralization assay was used to evaluate the potency and breadth of the neutralizing antibody response elicited by the different C4-V3 and gp120 immunogens against a spectrum of Glade-B viruses. All sera were tested against Bal, BX08, MN, SF-162, NL-43 and JR-CSF strains and all sera except C4-V3_{K305C-0321C} (SEQ ID NO: 30) were tested also against NSC. JR-CSF is known to be a neutralization resistant strain while all other strains are neutralization sensitive to various degrees. The phenotype of these HIV-1 strains are presented in Table 5, herein above.

All strains are R5 viruses with the exception of MN and NL-43 which utilize CXCR4. aMLV (Murine Leukemia Virus) was used as a negative control. As a further control, representative pre-immune sera were tested for neutralization of SF-162 and no neutralization activity was detected (data not shown). The neutralization results are summarized in Table 9, herein below.

TABLE 9

| | | | | | | | | NL- | |
|---|---|---|---|---|---|---|---|---|---|
| Rabbit | Immunogen | BaL | BX08 | MN | NSC | SF162 | JRCSF | 43 | aMLV |
| B712 | C4-V3L | <10 | <10 | <10 | 53 | 53 | <10 | <10 | <10 |
| B707 | C4-V3L | <10 | <10 | <10 | 11 | 63 | <10 | <10 | <10 |
| B702 | C4-V3L | 12 | 60 | 244 | 32 | 15 | <10 | 19 | <10 |
| B722 | C4-V3L | 15 | 31 | 38 | 62 | 964 | <10 | <10 | <10 |
| | GMT | <12 | <21 | <31 | 33 | <83 | N.N | N.N | N.N |
| B717 | C4- | <10 | 64 | <10 | 13 | 111 | <10 | <10 | <10 |
| | V3K305C- | | | | | | | | |
| | T320C | | | | | | | | |
| B715 | C4- | <10 | <10 | <10 | <10 | <10 | <10 | <10 | <10 |
| | V3K305C- | | | | | | | | |
| | T320C | | | | | | | | |
| B719 | C4- | <10 | 27 | 15 | 165 | 191 | <10 | 32 | <10 |
| | V3K305C- | | | | | | | | |
| | T320C | | | | | | | | |
| B723 | C4- | <10 | <10 | <10 | <10 | 14 | <10 | <10 | <10 |
| | V3K305C- | | | | | | | | |
| | T320C | | | | | | | | |
| | GMT | N.N | <20 | <11 | <22 | <42 | N.N | <13 | N.N |
| B866 | C4- | 12 | 15 | <10 | N.D | <10 | <10 | <10 | <10 |
| | V3K305C- | | | | | | | | |
| | G321C | | | | | | | | |
| B871 | C4- | <10 | <10 | <10 | N.D | <10 | <10 | <10 | <10 |
| | V3K305C- | | | | | | | | |
| | G321C | | | | | | | | |
| B872 | C4- | <10 | <10 | <10 | N.D | 25 | <10 | <10 | <10 |
| | V3K305C- | | | | | | | | |
| | G321C | | | | | | | | |
| B873 | C4- | 19 | 17 | 51 | N.D | 3379 | <10 | <10 | <10 |
| | V3K305C- | | | | | | | | |
| | G321C | | | | | | | | |
| | GMT | <12 | N.N | <15 | | <54 | N.N | N.N | N.N |
| B961 | C4- | 162 | 135 | 1958 | >5120 | >5120 | <10 | 597 | <10 |
| | V3T303C- | | | | | | | | |
| | E322C | | | | | | | | |
| B962 | C4- | 17 | 25 | 93 | 775 | 359 | <10 | 44 | <10 |
| | V3T303C- | | | | | | | | |
| | E322C | | | | | | | | |
| B963 | C4- | 60 | 61 | 1018 | >5120 | >5120 | <10 | 747 | <10 |
| | V3T303C- | | | | | | | | |
| | E322C | | | | | | | | |
| B964 | C4- | 57 | 46 | 474 | >5120 | 4939 | <10 | 269 | <10 |
| | V3T303C- | | | | | | | | |
| | E322C | | | | | | | | |
| | GMT | 56 | 55 | 544 | >3194 | >2612 | | 269 | |
| B716 | C4- | 12 | 27 | 30 | 1647 | 557 | <10 | <10 | <10 |
| | V3T303C- | | | | | | | | |
| | I323C | | | | | | | | |
| B720 | C4- | 111 | 285 | 1178 | 350 | >5120 | 22 | 10 | <10 |
| | V3T303C- | | | | | | | | |
| | I323C | | | | | | | | |
| B714 | C4- | 21 | 52 | 115 | 276 | 1150 | <10 | 42 | <10 |
| | V3T303C- | | | | | | | | |
| | I323C | | | | | | | | |
| B718 | C4- | 16 | 55 | 133 | 190 | 1900 | <10 | <10 | <10 |
| | V3T303C- | | | | | | | | |
| | I323C | | | | | | | | |
| | GMT | 26 | 68 | 152 | 417 | >1580 | N.N | <14 | N.N |
| B889 | C4- | 28 | 33 | 30 | 274 | 637 | <10 | <10 | <10 |
| | V3N301C- | | | | | | | | |
| | G325C | | | | | | | | |
| B890 | C4- | 39 | 38 | 582 | >5120 | 2412 | <10 | <10 | <10 |
| | V3N301C- | | | | | | | | |
| | G325C | | | | | | | | |
| B892 | C4- | 12 | 15 | 44 | 36 | 253 | <10 | <10 | <10 |
| | V3N301C- | | | | | | | | |
| | G325C | | | | | | | | |
| | GMT | 24 | 26 | 92 | >368 | 730 | | | |
| B955 | gp120 | 45 | 40 | 131 | 902 | 727 | <10 | <10 | <10 |
| B958 | gp120 | 14 | 18 | 64 | 438 | 540 | <10 | 15 | <10 |
| B959 | gp120 | 16 | 15 | 158 | 79 | 632 | <10 | <10 | <10 |
| B960* | gp120 | <10 | <10 | <10 | <10 | <10 | <10 | <10 | <10 |
| | GMT | 22 | 22 | 110 | 315 | 628 | | | |

^{a}Titers, calculated as the serum dilution conferring 50% inhibition (IC50) of pseudovirus infection are presented. The first measurement was done at 1:10 dilution and was followed by consecutive 2 fold dilutions. Geometric mean titer (GMT) is presented (<10 was calculated as 10).
N.D = not determined.
N.N = non-neutralizing.
*excluded from calculation due to lack of immune response in that rabbit.

The most effective immunogens were peptides constrained at position 303, of which C4-V3_{T303C-E322C} (SEQ ID NO: 31), representing the R5A conformation, induced the strongest neutralizing response. Both C4-V3_{T303C-E322C} (SEQ ID NO: 31) and C4-V3_{T303C-I323C} (SEQ ID NO: 2) immune sera neutralized the four R5 neutralization sensitive strains as well as MN which is an X4 strain. C4-V3_{T303C-E322C} (SEQ ID NO: 31) immune sera neutralized also the X4 neutralization-sensitive strain NL-43 and were the only immune sera that neutralized NL-43 significantly. It should be noted that NL-43 contains a rare two residue insertion preceding the GPGR segment. C4-V3_{T303C-E322C} (SEQ ID NO: 31) immune-sera were also significantly more effective than other sera in neutralizing the MN and NSC strains. For example the IC-90 GMT for NSC neutralization is over 12-fold higher for C4-V3_{T303C-E322C} (SEQ ID NO: 31) immune sera than for C4-V3_{T303C-I323C} (SEQ ID NO: 2) immune sera (Table 10, herein below).

TABLE 10

| Rabbit | Immunogen | MN | NSC | SF162 | NL-43 |
|---|---|---|---|---|---|
| B712 | C4-V3L | <10 | <10 | <10 | <10 |
| B707 | C4-V3L | <10 | <10 | <10 | <10 |
| B702 | C4-V3L | 23 | <10 | <10 | <10 |
| B722 | C4-V3L | <10 | 14 | 135 | <10 |
| | GMT | <12 | <11 | <19 | N.N |
| B961 | C4V3T303C-E322C | 149 | 2655 | 965 | 17 |
| B962 | C4V3T303C-E322C | 15 | 54 | 33 | <10 |
| B963 | C4V3T303C-E322C | 126 | 1484 | 1016 | 23 |
| B964 | C4V3T303C-E322C | 75 | 1204 | 498 | 16 |
| | GMT | 68 | 711 | 356 | <16 |
| B716 | C4V3T303C-I323C | <10 | 220 | 69 | <10 |
| B720 | C4V3T303C-I323C | 159 | 47 | 1111 | <10 |
| B714 | C4V3T303C-I323C | 19 | 37 | 223 | <10 |
| B718 | C4V3T303C-I323C | 20 | 29 | 196 | <10 |
| | GMT | <28 | 58 | 241 | N.N |
| B889 | C4-3N301C-G325C | <10 | 53 | 137 | <10 |
| B890 | C4-3N301C-G325C | 86 | 1110 | 341 | <10 |
| B892 | C4V3N301C-G325C | <10 | <10 | 40 | <10 |
| | GMT | <20 | <84 | 123 | N.N |
| B955 | gp120 | 21 | 89 | 112 | <10 |
| B958 | gp120 | 15 | 38 | 46 | <10 |
| B959 | gp120 | 20 | 15 | 73 | <10 |
| B960* | gp120 | <10 | <10 | <10 | <10 |
| | GMT | 18 | 37 | 72 | N.N |

^{a}Titers, calculated as the plasma dilution conferring 90% inhibition (IC90) are shown. Geometric mean titer (GMT) is presented (<10 was calculated as 10). N.N = non-neutralizing.
*excluded from calculation due to lack of immune response in that rabbit.

The IC-50 GMT for MN neutralization is 3.5-fold higher for C4-V3_{T303C-E322C} (SEQ ID NO: 31) then for C4-V3_{T303C-1323C} (SEQ ID NO: 2) immune sera (Table 10). Importantly, both peptides induced a much more potent neutralizing response then the linear C4-V3 peptide. Only one linear-peptide immune-serum neutralized 5 sensitive strains and two other C4-V3L (SEQ ID NO: 1) immune-sera neutralized only the two most neutralization sensitive strains. The neutralization titers were on average significantly lower for C4-V3L (SEQ ID NO: 1) immune sera than those elicited by peptide immunogens constrained at T303. For example, the IC-50 GMT of C4-V3L immune sera for NSC neutralization was more than one order of magnitude lower than that of the C4-V3_{T303C-1323C} immune sera and two orders of magnitude lower then that of C4-V3_{T303C-E322C} immune sera. Peptides constrained at K305 were found to be even less effective than the linear peptide in inducing antibodies capable of neutralizing HIV-1 isolates; none of the sera in the two groups of rabbits immunized by these peptides neutralized all sensitive strains tested, only one serum in each group neutralized more then two strains and the neutralization titers were much lower in most cases than those observed for the other constrained C4-V3 peptides. The peptide C4-V3_{N301C-G325C} (SEQ ID NO: 32) is constrained to assume the same R5B conformation as C4-V3_{T303C-1323C} (SEQ ID NO: 2) However, its disulfide bond is removed further away from the GPGR loop and the ring size enclosed by the disulfide bond is therefore four-residues larger. This peptide was included in order to test whether T303 is the optimal position for the disulfide constraint. C4-V3_{N301C-G325C} (SEQ ID NO: 32) induced a potent anti-gp120 and anti-V3 response which was more effective than that elicited by either the linear peptide or a peptide constrained at K305, nevertheless it was not as potent as C4-V3_{T303C-I323C} (SEQ ID NO: 2) and C4-V3_{T303C-E322C} (SEQ ID NO: 31) in eliciting an HIV-1 neutralizing response. For example, the GMT for IC-90 for SF-162 is 2 and 3 fold higher for the C4-V3_{T303C-1323C} (SEQ ID NO: 2) and C4-V3_{T303C-E322C} (SEQ ID NO: 31) immune sera in comparison with the C4-V3_{N301C-G325C} (SEQ ID NO: 32) immune sera. Only one out of three immune sera reached 90% inhibition for MN in the group immunized with C4-V3_{N301C-G325C} (SEQ ID NO: 32) compared with three out of four and four out of four for C4-V3_{T303C-1323C} (SEQ ID NO: 2) and C4-V3_{T303C-E322C} (SEQ ID NO: 31) immune sera, respectively. This demonstrates that T303 is indeed the optimal position for the disulfide bond constraint. gp120 elicited a relatively modest neutralizing response that resembles the antibody response elicited by C4-V3_{N301C-G325C} (SEQ ID NO: 32) and although it neutralized the two most sensitive strains only one serum neutralized all 5 sensitive strains that were tested. This supports the use of peptides based immunogens over gp120 based immunogens and demonstrates the benefit of optimally constraining the V3 loop to induce a more potent and cross-reactive neutralizing response.

Discussion

The study described in the present example evaluated the hypothesis that antibodies elicited by V3 peptides constrained to a conformation that mimics the native conformation of the V3 loop, will be more cross-reactive with gp120 and more potent in neutralizing HIV-1 in comparison with anti-sera elicited by linear V3 peptides. In the design of the peptides, the present inventors took into account the hydrogen bond network and the cross-strand alignment in order to mimic the β-hairpin conformation observed in the NMR and crystal structures of V3 peptides bound to the broadly neutralizing antibody 447-52D (see Example 1) and also in V3-containing gp120 (Huang et al., 2007 Science 317, 1930-1934; Huang et al., 2005, Science 310, 1025-1028). In this study, the location of the disulfide was changed and compared the gp120 cross reactivity and HIV-1 neutralizing response obtained by immunization with the disulfide-bond constrained-peptide with that obtained by a V3 linear immunogen and by gp120 with the goal of finding the optimally constrained V3-peptide immunogen.

Peptides constrained by a disulfide bond involving residue 303 were more effective immunogens for eliciting sera with gp120 cross-reactivity. The C4-V3_{T303C-E322C} (SEQ ID NO: 31) and C4-V3_{T303C-I323} (SEQ ID NO: 2) induced immune sera that showed the best cross-reactivity with gp120 with ratios of 1.06 and 1.1 between binding to gp120 and the corresponding V3 peptide, respectively (FIG. 9). These values indicate that the immune sera recognize gp120 as well as they recognize the V3 immunogen. The C4-V3_{K305C-T320C} (SEQ ID NO: 3) and C4-V3_{K305C-G321C} (SEQ ID NO: 30) immune sera exhibited slightly poorer cross-reactivity with gp120 of 0.69 and 0.80, respectively. This is despite the fact that V3_{K305C-T320C} (SEQ ID NO: 3) and V3_{K305C-G321C} (SEQ ID NO: 30) bind to 447-42D with affinities comparable to those of V3_{T303C-E322C} (SEQ ID NO: 31) or V3_{T303C-I323} (SEQ ID NO: 2), and with higher affinity compared with the consensus linear V3 peptide. It is possible that a subpopulation of the antibodies elicited by V3_{K305C-T320C} (SEQ ID NO: 3) and V3_{K305C-G321C} (SEQ ID NO: 30) interacted with the side chain of the cystine residue at position 305 and are sensitive to its replacement by other residues unlike 447-52D which is tolerant to replacements of amino acids in the β-strand. K305 interacted extensively with the antibody in the 447-52D complex with a V3_{MN} peptide and is one of the residues in the conserved triad K305-I307-I309 in the N-terminal strand of the V3 β-hairpin that was previously suggested to be important for the reactivity of gp120 with broadly neutralizing antibodies (Rosen et al, 2005, Biochemistry 44, 7250-7258). As a result non-conservative mutation of K305 could elicit antibodies that are less cross-reactive with gp120.

When the ring enclosed by the disulfide bond increases by four residues in comparison with V3_{T303C-I323C} (SEQ ID NO: 2) as in the peptide V3_{N301C-G325C} (SEQ ID NO: 32), the cross-reactivity with gp120 drops marginally from 1.1 to 0.82 (FIG. 9). Both V3_{T303C-I323C} (SEQ ID NO: 2) and V3_{N301C-G325C} (SEQ ID NO: 32) contain the entire 447-52D epitope with no modification of its residues. The present inventors suggest that the slight drop in the gp120 cross reactivity observed for the immune sera of the peptide with the larger ring size is due to the increased flexibility of the V3 segment in V3_{N301C-G325C} (SEQ ID NO: 32) in comparison with V3_{T303C-I323C} (SEQ ID NO: 2).

Peptides constrained at T303 are by far the most effective constrained peptide immunogens for eliciting an HIV-1 neutralizing response. Comparison of the neutralization efficiency of a panel of HIV-1 strains by the immune sera elicited by the different constrained peptide immunogens reveals that the peptides V3_{T303C-E322C} (SEQ ID NO: 31) and V3_{T303C-I323} (SEQ ID NO: 2) elicited a considerably more potent HIV-1 neutralization in comparison with peptides containing both shorter and longer ring sizes (i.e. V3_{K305C-T320C} (SEQ ID NO: 3), V3_{K305C-G321C} (SEQ ID NO: 30) and V3_{N301C-G325C} (SEQ ID NO: 32)). When neutralization of the neutralization-sensitive strain SF162 was compared among the immune-sera elicited by the different constrained-peptide immunogens, it was found that the neutralization efficiency of the immune-sera increased by approximately 30-fold when the disulfide bond was moved from position 305 to 303. When the disulfide bond was located further away from the GPGR segment, i.e. involving position 301, the neutralization efficiency dropped by two fold, when the immune sera of C4-V3_{T303C-I323C} (SEQ ID NO: 2) and C4-V3_{N301C-G325C} (SEQ ID NO: 32) are compared, indicating that the optimal constraint is a disulfide bond involving residue 303.

It is surprising that C4-V3_{K305C-T320C} (SEQ ID NO: 3) and C4-V3_{K305C-G321C} (SEQ ID NO: 30) elicited poorer HIV-1 neutralizing responses in comparison with C4-V3_{T303C-E322C} (SEQ ID NO: 31) and C4-V3_{T303C-I323C} (SEQ ID NO: 2) although the differences in gp120 cross-reactivity were not pronounced. Moreover, although the immune sera of peptides constrained at residue 305 bound gp120 better than V3L, these sera were poorer in HIV-1 neutralization. A possible explanation is that both C4-V3_{T303C-E322C} (SEQ ID NO: 31) and C4-V3_{T303C-I323C} (SEQ ID NO: 2) elicited larger fraction of the high-affinity antibodies that are crucial for HIV-1 neutralization. Replacement of residues within the epitope recognized by the elicited HIV-1 neutralizing antibodies (such as happens when cysteine residues within the epitopes are used to constrain the peptides) or increased flexibility may lower the population of the high-affinity antibodies and result in more dramatic differences in HIV-1 neutralization in comparison with the differences observed in gp120 cross-reactivity. It is also possible that the V3 region in the native virions is considerably less flexible than in the monomeric gp120 construct used in the binding assays, so that a larger difference in binding cross-reactivity would be observed if binding to HIV-1 virions had been examined.

The linear consensus V3 peptide elicits antibodies that cross react with gp120 but have a much lower HIV-1 neutralization potency than the immune sera induced by V3-peptides constrained at T303. C4-V3L (SEQ ID NO: 1) elicited antibodies that were surprisingly highly cross-reactive with gp120 and the ratio between gp120 and V3L binding was 0.54. This ratio is only a factor of two lower than that observed for the immune-sera of peptides constrained by a disulfide bond involving residue 303. It is known that the GPGR segment in V3 peptides transiently populates a β-turn conformation. Thus although it does not form a β-hairpin like conformation V3L peptide is not completely flexible. The β-turn forming GPGR segment is the core epitope for many V3-directed HIV-1 neutralizing antibodies and, for example, in the V3_{MN} complex with 447-52D the GPGR segment occupies a central pocket in the antibody binding site. The population in V3L of a β-turn conformation that is similar to that found in native gp120 may be the reason that this peptide elicits a high proportion of gp120-cross-reactive antibodies which neutralize HIV-1. However, the present comparative study of different V3 immunogens showed clearly that peptides constrained at position 303 elicited at least a 20-fold more potent HIV-1 neutralizing response in comparison with V3L using SF-162 as the virus in the neutralization assay.

Peptides constrained at position 303 elicit a better HIV-1 neutralizing response than gp120. The HIV-1 neutralization elicited by gp120 in the present study is considerably poorer than that elicited by peptides constrained by a disulfide bond involving residue 303. For example, when neutralization of SF-162 or NSC is compared, the C4-V3_{T303C-E322C} (SEQ ID NO: 31) immune sera was 5-fold or 19-fold, respectively more potent than the gp120 immune sera (Table 10), and the gp120 induced sera did not neutralize NL-43 at all (Table 9). The poorer HIV-1 neutralization by the gp120 immune sera was obtained despite the fact that the V3 region was fully exposed in the truncated gp120 construct used in the present study. These findings imply that gp120 determinants other than the V3 loop elicit only a weak HIV-1 neutralizing response in comparison with the optimal V3 peptide and that the V3 directed HIV-1 neutralizing antibodies elicited by the V3-containing gp120 are not as potent as those elicited by the optimal C4-V3_{T303-E322C} (SEQ ID NO: 31) and C4-V3_{T303-I323C} (SEQ ID NO: 2) peptides.

The C4-V3_{T303C-E322C} (SEQ ID NO: 31) and C4-V3_{T303C-1323C} (SEQ ID NO: 2) immune sera are broadly neutralizing. The panel of the HIV-1 strains tested for neutralization by the immune sera contains four Glade-B strains that are highly sensitive to neutralization, i.e. MN, NSC, SF162 and NL43. None of these strains contains a sequence identical to the immunizing consensus V3 peptide. The MN strain contains the mutations T303K, S306R, G321K and D322N. The NSC strain contains the mutations K305R, H308T and I309M. The SF162 contains the mutations H308T, T319A and E322D. The NL-43 strain that resembles HIV-1_{IIIB} is the most distant from the immunizing peptide and in addition to four mutations (H308R, Y318V, T320I and E322K) it contains a two residue insertion (Q310-R311) and one deletion (I323). These differences in the V3 sequences of the tested HIV-1 strains and especially the large differences between the NL-43 strain and the immunizing peptide indicate that the immune sera generated by the C4-V3_{T303C-E322C} (SEQ ID NO: 31) peptide is highly cross-reactive with Glade-B viruses when their V3 is exposed and they contain the GPGR motif. Moreover the C4-V3_{T303C-E322C} (SEQ ID NO: 31) immune sera were capable of neutralizing both X4 and R5 viruses.

The C4-V3_{T303C-E322C} (SEQ ID NO: 31) peptide elicits the most potent HIV-1 neutralization The immune sera elicited by the two peptides C4-V3_{T303C-E322C} (SEQ ID NO: 31) and C4-V3_{T303C-I323C} (SEQ ID NO: 2) constrained by a disulfide bond involving residue 303 both exhibited high levels of cross-reactivity with gp120. However C4-V3_{T303C-E322C} (SEQ ID NO: 31) elicited a more potent HIV-1 neutralizing response in comparison with C4-V3_{T303C}-(_{323C} (SEQ ID NO: 2) for all the strains tested except BX08. Especially large differences were observed for the NSC strain, which was neutralized more than 7-fold better by the C4-V3_{T303C-E322C} (SEQ ID NO: 31) immune sera, and for the NL-43 strain that was practically not neutralized by the C4-V3_{T303C-I323C} (SEQ ID NO: 2) immune sera but was very well neutralized by the C4-V3_{T303C-E322C} (SEQ ID NO: 31) immune sera. C4-V3_{T303C-E322C} (SEQ ID NO: 31) was constrained to the postulated R5A conformation of the V3 loop while C4-V3_{T303C-I323C} (SEQ ID NO: 2) was constrained to the R5B conformation. These two conformations are identical in the register of the hydrogen forming residues in the V3 N-terminal β-strand however they differ in the pairing of the residues in the β-hairpin. Moreover, C4-V3_{T303C-E322C} (SEQ ID NO: 31) is similar to the postulated X4 V3 conformation in the pairing of the residues. This resemblance could explain the substantial increase in neutralization potency of X4 viruses (HIV-1_{MN} and HIV-1_{NL43}) by the C4-V3_{T303C-E322C} (SEQ ID NO: 31) immune sera.

Conclusions

In the present study the C4-V3_{T303C-E322C} (SEQ ID NO: 31) immunogen elicited a potent HIV-1 neutralizing response that was at least 30 fold stronger than that elicited by a linear C4-V3 immunogen when neutralization of SF-162 was compared and 100-fold stronger when neutralization of the NSC strain is compared. The C4-V3_{T303C-E322C} (SEQ ID NO: 31) immune-sera were on average also 4-fold more potent than the gp120 immune sera when neutralization of SF-162 strain was compared, 10-fold stronger when neutralization of the NSC strain was compared and was also able to neutralize the NL-43 strain. The C4-V3_{T303C-E322C} (SEQ ID NO: 31) immune sera exhibited broad neutralization within Glade-B viruses in which the V3 was exposed.

Comparison with the other constrained V3 immunogens lead to the following conclusions:

a) It is important to include the intact K305-T320 segment of V3 without replacement to cysteine.

b) High affinity binding to 447-52D can be misleading in selecting an optimal immunogen.

c) It may not be necessary to achieve a rigid β-hairpin conformation in the peptide immunogen and some flexibility may be beneficial for eliciting a strong HIV-1 neutralizing response.

d) Too much flexibility in the immunogen is detrimental to achieving broad HIV-1 neutralization.

e) The ring enclosed by the disulfide bond should be minimal while fully containing the intact K305-T320 determinant.

### Example 3

### Synergism of Anti-V3 Antibodies with the CD4-Mimic Peptide CD4M33

Materials and Methods

Determination of Synergy

In order to asses the combination effect of sera immunized with C4-V3_{T303C-I323C} (SEQ ID NO: 2) and the CD4 mimic peptide CD4M33, neutralization was tested for the serum alone starting at 1:10 with consecutive 2-fold dilution (total of 10 points), and similarly for CD4M33 alone starting at a concentration that results in approximately 50-75% inhibition (0.25 μg/ml for Bal, 0.4 μg/ml for BX08 and 1.25 μg/ml for 6535). In a third neutralization experiment, the combination of the two in a constant ratio was generated; for example for BX08 1:10 serum dilution with 0.4 μg/ml of CD4M33 was used as the starting concentration for the 10 2-fold dilutions curve.

Combination index (CI) was calculated using CalcuSyn software version 2.0 (Biosoft, Cambridge, UK) according to the model by Chou and Talalay (Adv Enzyme Regul 22, 27-55) assuming mutually exclusive assumption (similar modes of action) whereas CI<1, =1 and >1 indicates synergism, additive effect and antagonism, respectively. The general equation for CI is given by: CI=(D)₁/(Dₓ)₁+(D)₂/(D_{R})₂. (Dₓ)₁ is the concentration of drug #1, for example serum dilution (1:titer, which is propositional to the antibody concentration), (Dₓ)₂ is the concentration of drug #2, for example CD4M33 peptide (μg/ml) that inhibits by x percent when used alone. (D)₁ and (D)₂ are the concentrations of drug #1 and drug #2 in combination that also inhibits by x percent. In cases where the serum alone was not neutralizing, the data can be presented by the reduction in the concentration of CD4M33 needed to achieve an indicated percent of neutralization in the presents of the serum compared with CD4M33 alone, i.e. Dose Reduction Index (DRI). For simplicity, the reciprocal of the DRI (i.e. the concentration of the CD4M33 in the presence of the serum divided by the concentration in the absence of the serum) is presented, so that values <1 indicates an enhancement in neutralization.

A classical isobologram was generated by the software for mutually exclusive mode of action. For a given percent of neutralization the CD4M33 concentration and serum dilution is drawn on the x-axis and y-axis, respectively. A point indicating the serum dilution and CD4M33 concentration in the combination is drawn. If the data point falls on the diagonal an additive effect is indicated. Appoint above or below the line indicates antagonism and synergism respectively.

Results

The V3 region is occluded in neutralization resistant primary isolates and therefore such viruses are not sensitive to neutralization by anti-V3 antibodies. sCD4, which causes a conformational change in gp120 that exposes the V3, was found to broaden the neutralization profile of some anti-V3 antibodies (Wu et al., Virology 380, 285-95). Since the 27-residue CD4-mimic peptide CD4M33 was found to induce the CD4 bound gp120 conformation similar to sCD4, as a proof of principle for other CD4 mimic compounds such as the small-molecule NBD-556 and its analogues, the present inventors tested whether CD4M33 can work in synergy with the C4-V3_{T303C-I323C} (SEQ ID NO: 2) immune-sera to broaden the repertoire of HIV strains neutralized.

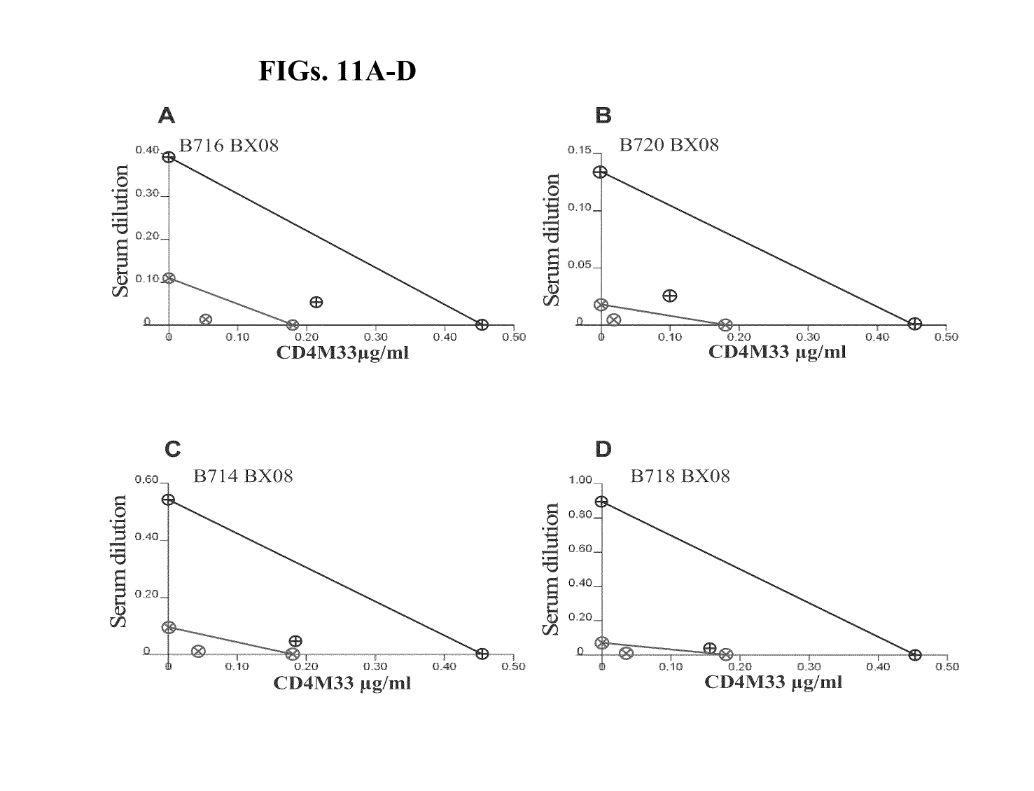

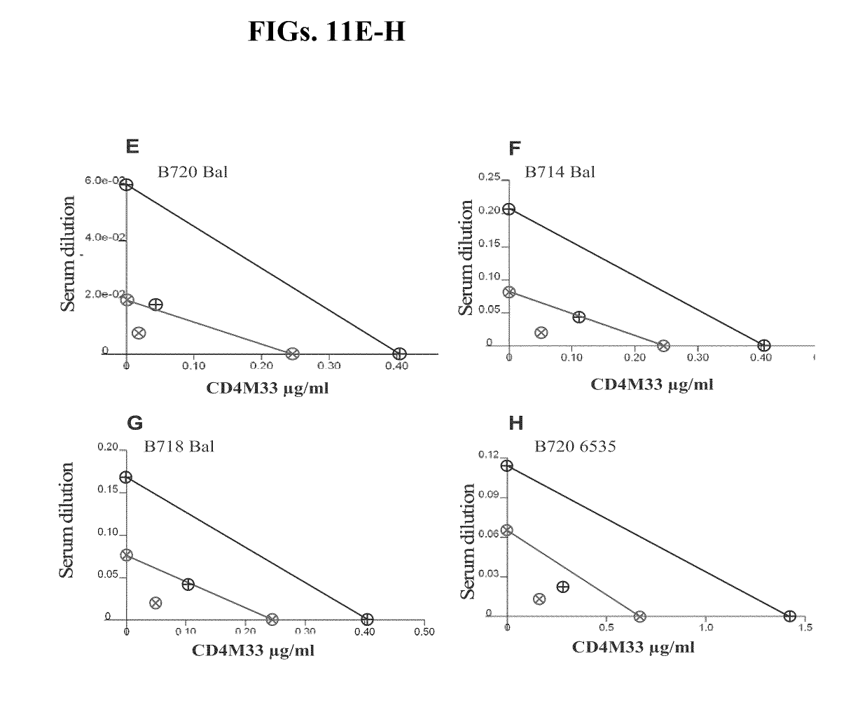

The neutralization of several HIV-1 strains that are moderately sensitive to neutralization was tested with the C4-V3_{T303C-I323C} (SEQ ID NO: 2) immune sera, with CDM33 and with combination of the two. Good synergism was observed for Bal and BX08 and for one of the rabbits immune sera also in HIV-1-6535 neutralization. The data and its analysis are summarized in FIG. 11 and Tables 11 and 12, herein below. For Table 11, the concentration of CD4M33 peptide and serum titer of C4-V3_{T303C-I323C} (SEQ ID NO: 2) immune-sera that achieved 50% inhibition (IC-50) in the pseudo-virus infection assay either separately or in combination is shown.

Table 12 demonstrates the synergism between sera of rabbits immunized with C4-V3_{T303C-I323C} (SEQ ID NO: 2) and CD4M33 in neutralization of HIV-1 pseudovirus infection. Specifically, the combination index (C.I) at 50% or 75% neutralization as determined by CalcuSyn software of viral strains for sera of rabbits immunized with C4-V3_{T303C-I323C} and CD4M33 is shown.

TABLE 11

| | HIV-1 strain | | | | | |
|---|---|---|---|---|---|---|
| | BX08 | | Bal | | 6535 | |
| | Serum | CD4M33 | Serum | CD4M33 | Serum | CD4M33 |
| Compounds | (1/dilution) | (μg/ml) | (1/dilution) | (μg/ml) | (1/dilution) | (μg/ml) |
| CD4M33 | | 0.173 | | >0.25 | | 0.721 |
| B716 | <10 | — | <10 | 0.000 | <10 | — |
| B716 + CD4M33 | 111 | 0.036 | 44 | 0.057 | 29 | 0.431 |
| B720 | 58 | — | 52 | — | 15 | — |
| B720 + CD4M33 | 286 | 0.014 | 144 | 0.017 | 66 | 0.189 |
| B714 | 12 | — | 13 | — | <10 | — |
| B714 + CD4M33 | 111 | 0.036 | 52 | 0.048 | 29 | 0.431 |
| B718 | 11 | — | 15 | — | <10 | — |
| B718 + CD4M33 | 141 | 0.028 | 55 | 0.045 | 26 | 0.481 |

TABLE 13

CI at % Neutralization of

| Serum | Strain | 50% | 75% |
|---|---|---|---|
| B716 | BX08 | 0.42 | 0.61 |
| B720 | | 0.34 | 0.41 |
| B714 | | 0.35 | 0.49 |
| B718 | | 0.32 | 0.39 |
| B716 | Bal | 0.25* | 0.33* |
| B720 | | 0.46 | 0.40 |
| B714 | | 0.46 | 0.49 |
| B718 | | 0.46 | 0.50 |
| B716 | 6535 | 0.71* | 0.57* |
| B720 | | 0.44 | 0.40 |
| B714 | | 0.75* | 0.56* |
| B718 | | 0.78* | 0.58* |

*In cases where no neutralization was observed for the serum alone, the reciprocal of the Dose Reduction Index (DRI) is shown.

Table 12 presents the titers (i.e. 1/serum dilution) at which 50% neutralization of the BX08, Bal and 6536 strains is obtained by the immune sera of the four rabbits: B716, B720, B714 and B718. (Thus, for example, when the dilution is 1 the serum concentration is 100%. When the dilution is 0.5, the serum concentration is 50%) The CD4M33 concentration that results in 50% neutralization is provided as well. The starting solution in these experiments contained the sera in 1:10 dilution together with a CD4 concentration that caused significant neutralization of the tested strains (76% for BX08, 50% for Bal and 74% for 6535). This combination of the immune sera and CD4 went into serial 2-fold dilutions, and neutralization was measured at each step. The titer of the sera and the CD4M33 concentration in this combination solution that results in 50% neutralization of BX08, Bal and 6535 are listed in Table 12. The calculated values for the synergism given as combination number calculated by the CalcuSyn program are presented in Table 13. For the cases where both the sera and the CD4M33 reached significant neutralization alone and CI values can be calculated, the CI values ranged from 0.32-0.46 to achieve a 50% inhibition and from 0.39-0.61 to achieve a 75% inhibition, indicating good synergism between the C4-V3-peptide immune sera and CD4M33 (Table 13). In cases where the serum alone was not neutralizing, the reciprocal of the Dose Reduction Index (DRI) is shown. Values >1 indicate an enhancement effect on neutralization. The 1/DRI for the one sera that did not neutralize Bal (B716) was 0.25 and 0.33 for 50% and 75% inhibition respectively. For 6535, the values range from 0.56-0.78 indicating a moderately positive effect.

A graphic illustration for the synergism between the immune sera and CD4M33 is provided in FIGS. 11A-H. The X and Y axis present the CD4M33 concentration and serum dilution respectively. A straight line was drawn between the two points representing the serum dilution and CD4M33 concentration that result in 50% and another line was drawn for 75% neutralization. The concentration for the serum and the CD4M33 in the combined solution that gave 50% and 75% neutralization are given by the X and +sign respectively. The location of this sign below the straight line provides an indication for the synergism. Thus, if there was no synergism the +sign was on the 75% neutralization line and the X sign was on the 50% line. As can be seen in FIGS. 11A-H, the serum dilution and CD4M33 concentration that would result in approximately 50% neutralization assuming additivity, in fact resulted in about 75% neutralization due to the synergism as indicated by the location of the +point close to or below the diagonal of the 50% neutralization. Overall these data demonstrate that the CD4 mimic peptide can expose the V3 loop in those strains and neutralize HIV-1 in synergy with the C4-V3_{T303C-I323C} (SEQ ID NO: 2) immune sera. The strains QH0692, JR-CSF APV-18 and NL-43 were also tested, but the results obtained did not point to synergy. NL-43 is an X4 neutralization sensitive strain that was not recognized by the elicited antibodies probably due to the unusual QR insertion. APV-18 has a R315K mutation which probably accounts for the inability of the antibodies elicited by C4-V3_{T303C-I323C} (SEQ ID NO: 2) to recognize this strain. For QH0692 and JR-CSF there is no mutation in the V3 sequence that can explain the lack of synergy but these two strains are more neutralization resistant than BX08, Bal and 6535.

Taken as a whole, the present data indicated that CD4M33 can expose the V3 loop similarly to sCD4.

Conclusions

The present inventors have demonstrated that the CD4-mimic compound CD4M33 acts in synergy with antibodies elicited against constrained-V3 peptides immunogens to neutralize representative HIV-1 viruses. sCD4 was found to broaden the spectrum of HIV-1 strains neutralized by V3-directed antibodies (Wu, 2008 Virology 380, 285-95). The present inventors suggest that CD4M33 and the small molecule NBD-556 and its analogues can act similarly. These small molecules can be administered orally or as microbicides to people that have been already immunized with the constrained V3-peptides as a pre-exposure prophylaxis or short time post exposure. When encountered by the virus, these CD4-mimic molecules will bind to the gp120 and induce the conformational change that exposes the V3 and make it vulnerable to the vaccine-elicited anti-V3 antibodies. The antibodies elicited by the constrained peptide, when present at high enough concentration will bind to the V3 and neutralize the HIV-1 virus, thereby prevent infection.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCE

(1) Rusche, J. R., Javaherian, K., McDanal, C., Petro, J., Lynn, D. L., Grimaila, R., Langlois, A., Gallo, R. C., Arthur, L. 0., Fischinger, P. J., and et al. Antibodies that inhibit fusion of human immunodeficiency virus-infected cells bind a 24-amino acid sequence of the viral envelope, gp120. Proc Natl Acad Sci USA 85, 3198-202 (1988).
(2) Wu, L., Gerard, N. P., Wyatt, R., Choe, H., Parolin, C., Ruffing, N., Borsetti, A., Cardoso, A. A., Desjardin, E., Newman, W., Gerard, C., and Sodroski, J. CD4-induced interaction of primary HIV-1 gp120 glycoproteins with the chemokine receptor CCR-5. Nature 384, 179-83. (1996).
(3) Trkola, A., Dragic, T., Arthos, J., Binley, J. M., Olson, W. C., Allaway, G. P., Cheng-Mayer, C., Robinson, J., Maddon, P. J., and Moore, J. P. CD4-dependent, antibody-sensitive interactions between HIV-1 and its co-receptor CCR-5. Nature 384, 184-7. (1996).
(4) Liao, H. X., Etemad-Moghadam, B., Montefiori, D.C., Sun, Y., Sodroski, J., Scearce, R. M., Doms, R. W., Thomasch, J. R., Robinson, S., Letvin, N. L., and Haynes, B. F. Induction of antibodies in guinea pigs and rhesus monkeys against the human immunodeficiency virus type 1 envelope: neutralization of nonpathogenic and pathogenic primary isolate simian/human immunodeficiency virus strains. J Virol 74, 254-63. (2000).
(5) Letvin, N. L., Robinson, S., Rohne, D., Axthelm, M. K., Fanton, J. W., Bilska, M., Palker, T. J., Liao, H. X., Haynes, B. F., and Montefiori, D.C. Vaccine-elicited V3 loop-specific antibodies in rhesus monkeys and control of a simian-human immunodeficiency virus expressing a primary patient human immunodeficiency virus type 1 isolate envelope. J Virol 75, 4165-75. (2001).
(6) Someya, K., Cecilia, D., Ami, Y., Nakasone, T., Matsuo, K., Burda, S., Yamamoto, H., Yoshino, N., Kaizu, M., Ando, S., Okuda, K., Zolla-Pazner, S., Yamazaki, S., Yamamoto, N., and Honda, M. Vaccination of rhesus macaques with recombinant Mycobacterium bovis bacillus Calmette-Guerin Env V3 elicits neutralizing antibody-mediated protection against simian-human immunodeficiency virus with a homologous but not a heterologous V3 motif. J Virol 79, 1452-62 (2005).
(7) Hewer, R., and Meyer, D. Evaluation of a synthetic vaccine construct as antigen for the detection of HIV-induced humoral responses. Vaccine 23, 2164-7 (2005).
(8) Golding, B., Eller, N., Levy, L., Beining, P., Inman, J., Matthews, N., Scott, D. E., and Golding, H. Mucosal immunity in mice immunized with HIV-1 peptide conjugated to Brucella abortus. Vaccine 20, 1445-50 (2002).
(9) Haynes, B. F., Ma, B., Montefiori, D.C., Wrin, T., Petropoulos, C. J., Sutherland, L. L., Scearce, R. M., Denton, C., Xia, S. M., Korber, B. T., and Liao, H. X. Analysis of HIV-1 subtype B third variable region peptide motifs for induction of neutralizing antibodies against HIV-1 primary isolates. Virology 345, 44-55 (2006).
(10) Zvi, A., Hiller, R., and Anglister, J. Solution conformation of a peptide corresponding to the principal neutralizing determinant of HIV-1 IIIB: a two-dimensional NMR study. Biochemistry 31, 6972-9 (1992).
(11) Chandrasekhar, K., Profy, A. T., and Dyson, H. J. Solution conformational preferences of immunogenic peptides derived from the principal neutralizing determinant of the HIV-1 envelope glycoprotein gp120. Biochemistry 30, 9187-94 (1991).
(12) Matsushita, S., Robert Guroff, M., Rusche, J., Koito, A., Hattori, T., Hoshino, H., Javaherian, K., Takatsuki, K., and Putney, S. Characterization of a human immunodeficiency virus neutralizing monoclonal antibody and mapping of the neutralizing epitope. J Virol 62, 2107-14 (1988).
(13) Gorny, M. K., Xu, J. Y., Karwowska, S., Buchbinder, A., and Zolla-Pazner, S. Repertoire of neutralizing human monoclonal antibodies specific for the V3 domain of HIV-1 gp120. J Immunol 150, 635-43. (1993).
(14) Gorny, M. K., Revesz, K., Williams, C., Volsky, B., Louder, M. K., Anyangwe, C. A., Krachmarov, C., Kayman, S. C., Pinter, A., Nadas, A., Nyambi, P. N., Mascola, J. R., and Zolla-Pazner, S. The v3 loop is accessible on the surface of most human immunodeficiency virus type 1 primary isolates and serves as a neutralization epitope. J Virol 78, 2394-404 (2004).
(15) Binley, J. M., Wrin, T., Korber, B., Zwick, M. B., Wang, M., Chappey, C., Stiegler, G., Kunert, R., Zolla-Pazner, S., Katinger, H., Petropoulos, C. J., and Burton, D. R. Comprehensive cross-clade neutralization analysis of a panel of anti-human immunodeficiency virus type 1 monoclonal antibodies. J Virol 78, 13232-52 (2004).
(16) Rosen, 0., Chill, J., Sharon, M., Kessler, N., Mester, B., Zolla-Pazner, S., and Anglister, J. Induced fit in HIV-neutralizing antibody complexes: evidence for alternative conformations of the gp120 V3 loop and the molecular basis for broad neutralization. Biochemistry 44, 7250-8 (2005).
(17) Sharon, M., Kessler, N., Levy, R., Zolla-Pazner, S., Gorlach, M., and Anglister, J. Alternative Conformations of HIV-1 V3 Loops Mimic beta Hairpins in Chemokines, Suggesting a Mechanism for Coreceptor Selectivity. Structure 11, 225-236 (2003).
(18) Tugarinov, V., Zvi, A., Levy, R., and Anglister, J. A cis proline turn linking two beta-hairpin strands in the solution structure of an antibody-bound HIV-1(IIIB) V3 peptide. Nature Structural Biology 6, 331-335 (1999).
(19) Tugarinov, V., Zvi, A., Levy, R., Hayek, Y., Matsushita, S., and Anglister, J. NMR structure of an anti-gp120 antibody complex with a V3 peptide reveals a surface important for co-receptor binding [In Process Citation]. Structure Fold Des 8, 385-95 (2000).
(20) Hutchinson, E. G., Sessions, R. B., Thornton, J. M., and Woolfson, D. N. Determinants of strand register in antiparallel beta-sheets of proteins. Protein Sci 7, 2287-300 (1998).
(21) Chakraborty, K., Durani, V., Miranda, E. R., Citron, M., Liang, X., Schleif, W., Joyce, J. G., and Varadarajan, R. Design of immunogens that present the crown of the HIV-1 V3 loop in a conformation competent to generate 447-52D like antibodies. Biochem J (2006).
(22) Ratner, L., Haseltine, W., Patarca, R., Livak, K. J., Starcich, B., Josephs, S. F., Doran, E. R., Rafalski, J. A., Whitehorn, E. A., Baumeister, K., and et al. Complete nucleotide sequence of the AIDS virus, HTLV-III. Nature 313, 277-84 (1985).
(23) Braunschweiler, L., and Ernst, R. R. Coherence transfer of Isotropic mixing: application to proton correlation spectroscopy. J Magn Reson 53, 521-528 (1983).
(24) Shaka, A. J., Keeler, J., and Freeman, R. Evaluation of a new broadband decoupling sequence: WALTZ-16. Journal of Magnetic Resonance 53, 313-340 (1983).
(25) Rucker, S. P., Shaka, A. J. Broadband homonuclear cross polarization in 2D NMR using DIPSI-2. Mol. Phys. 68, 509-517 (1989).
(26) Piantini, U., Sorensen, O., Ernst, R. R. J. Am. Chem. Soc. 104, 6800-6801 (1982).
(27) Sklenar, V., Piotto, M., Leppik, R., and Saudek, V. Gradient-Tailored Water Suppression For H-1-N-15 Hsqc Experiments Optimized to Retain Full Sensitivity. Journal of Magnetic Resonance Series a 102, 241-245 (1993).
(28) Piotto, M., Saudek, V., and Sklenar, V. Gradient-tailored excitation for single-quantum NMR spectroscopy of aqueous solutions. J Biomol Nmr 2, 661-5 (1992).
(29) Hwang, T. L., and Shaka, A. J. Water Suppression That Works. Excitation Sculpting Using Arbitrary Wave-Forms and Pulsed-Field Gradients. Journal of Magnetic Resonance, Series A 112, 275-279 (1995).
(30) Delaglio, F., Grzesiek, S., Vuister, G. W., Zhu, G., Pfeifer, J., and Bax, A. NMRPipe: a multidimensional spectral processing system based on UNIX pipes. J Biomol NMR 6, 277-93 (1995).
(31) Johnson, B. A. Using NMRView to visualize and analyze the NMR spectra of macromolecules. Methods Mol Biol 278, 313-52 (2004).
(32) Neidig, K. P., Geyer, M., Gorler, A., Antz, C., Saffrich, R., Beneicke, W., and Kalbitzer, H. R. Aurelia, a Program For Computer-Aided Analysis of Multidimensional Nmr-Spectra. Journal of Biomolecular Nmr 6, 255-270 (1995).
(33) Wuthrich, K. NMR of proteins and nucleic acids, John Wiley, New York (1986).
(34) Herrmann, T., Guntert, P., and Wuthrich, K. Protein NMR structure determination with automated NOE-identification in the NOESY spectra using the new software ATNOS. Journal of Biomolecular Nmr 24, 171-189 (2002).
(35) Roberts, G. C. K. NMR of macromolecules, a practical approach, Oxford university press, New York (1993).
(36) Reeves, P. J., Callewaert, N., Contreras, R., and Khorana, H. G. Structure and function in rhodopsin: high-level expression of rhodopsin with restricted and homogeneous N-glycosylation by a tetracycline-inducible N-acetylglucosaminyltransferase I-negative HEK293S stable mammalian cell line. Proc Natl Acad Sci USA 99, 13419-24 (2002).
(37) Richman, D. D., Wrin, T., Little, S. J., and Petropoulos, C. J. Rapid evolution of the neutralizing antibody response to HIV type 1 infection. Proc Natl Acad Sci USA 100, 4144-9 (2003).
(38) Rosen, 0., Sharon, M., Quadt-Akabayov, S. R., and Anglister, J. Molecular switch for alternative conformations of the HIV-1 V3 region: implications for phenotype conversion. Proc Natl Acad Sci USA 103, 13950-5 (2006).
(39) Robey, F. A., Kelson-Harris, T., Roller, P. P., and Robert-Guroff, M. A helical epitope in the C4 domain of HIV glycoprotein 120. J Biol Chem 270, 23918-21 (1995).
(40) Palker, T. J., Matthews, T. J., Langlois, A., Tanner, M. E., Martin, M. E., Scearce, R. M., Kim, J. E., Berzofsky, J. A., Bolognesi, D. P., and Haynes, B. F. Polyvalent human immunodeficiency virus synthetic immunogen comprised of envelope gp120 T helper cell sites and B cell neutralization epitopes. J Immunol 142, 3612-9 (1989).
(41) Streeck, H., Schweighardt, B., Jessen, H., Allgaier, R. L., Wrin, T., Stawiski, E. W., Jessen, A. B., Allen, T. M., Walker, B. D., and Altfeld, M. Loss of HIV-1-specific T-cell responses associated with very rapid HIV-1 disease progression. Aids 21, 889-91 (2007).

1. An isolated cyclic polypeptide comprising a single internal constraint, comprising at least eighteen consecutive amino acids of a V3 domain of gp120, starting at position 303 and ending at position 322, said positioning being according to a numbering of said V3 domain of gp120 in a HXB2 strain, wherein amino acids at position 303 and 322 are bonded to each other.

2. An isolated cyclic polypeptide comprising at least nineteen consecutive amino acids of a V3 domain of gp120, starting at position 303 and ending at position 323, said positioning being according to a numbering of said V3 domain of gp120 in a HXB2 strain, wherein amino acids at position 303 and 323 are bonded to each other.

3-4. (canceled)

5. The isolated cyclic polypeptide of claim 2, wherein amino acids at position 303 and 323 are cysteines.

6. The isolated cyclic polypeptide of claim 1, wherein amino acids at position 303 and 322 are cysteines.

7. (canceled)

8. The isolated cyclic polypeptide of claim 1, wherein an amino acid at position 312 is glycine, an amino acid at position 313 is proline and an amino acid at position 314 is glycine.

9. The isolated cyclic polypeptide of claim 1, comprising a single internal disulfide bond.

10. The isolated cyclic polypeptide of any claim 1, wherein an amino acid at position 315 is arginine lysine or glutamine.

11. The isolated cyclic polypeptide of claim 1, wherein an amino acid at position 305 is lysine or arginine, and an amino acid at position 307 is isoleucine, leucine or valine and an amino acid at position 309 is isoleucine, leucine, methionine or valine

12. The isolated cyclic polypeptide of claim 1, wherein an amino acid at positions 319 and 320 are threonine or alanine.

13. The isolated cyclic polypeptide of claim 2, as set forth in SEQ ID NO: 2 or SEQ ID NO: 27.

14. (canceled)

15. The isolated cyclic polypeptide of claim 1, as set forth in SEQ ID NO: 31, 33 or 39.

16. (canceled)

17. The isolated cyclic polypeptide of claim 1, consisting of naturally occurring amino acids.

18. The isolated cyclic polypeptide of claim 1, further comprising amino acids of an antigen presenting polypeptide.

19. An isolated cyclic polypeptide comprising an amino acid consensus sequence X₁X₂X₃X₄X₅X₆X₇X₈X₉X₁₀X₁₁X₁₂X₁₃X₁₄X₁₅X₁₆X₁₇X₁₈X₁₉, where X₁ and X₁₉ are bonded, X₈ is glycine, X₉ is proline and X₁₀ is glycine.

20. An isolated cyclic polypeptide comprising
(i) an amino acid consensus sequence X₁X₂X₃X₄X₅X₆X₇X₈X₉X₁₀X₁₁X₁₂X₁₃X₁₄X₁₅X₁₆X₁₇X₁₈, where X₁ and X₁₈ are bonded, X₈ is glycine, X₉ is proline and X₁₀ is glycine, the polypeptide comprising a single internal constraint.

21-30. (canceled)

31. The isolated cyclic polypeptide of claim 1, comprising an amino acid sequence of a T-helper epitope.

32-35. (canceled)

36. The isolated cyclic polypeptide of claim 31, wherein said T-helper epitope comprises amino acids of HIV p24 gag.

37. The isolated cyclic polypeptide of claim 31, wherein said T-helper epitope comprises an amino acid sequence as set forth in SEQ ID NOs: 25 or 26.

38-40. (canceled)

41. The isolated cyclic polypeptide of claim 31, being no more than 50 amino acids.

42. A vaccine comprising the polypeptide of claim 1, as an active agent and an immunologically acceptable carrier.

43. (canceled)

44. An article of manufacture comprising the vaccine of claim 42 and a CD4 mimic compound.

45-48. (canceled)

49. A method of generating an immune response against HIV in a individual, the method comprising administering to the individual an effective amount of a V3 peptide-based vaccine and further comprising administering to the individual an effective amount of a CD4 mimic compound.

50-52. (canceled)

53. An isolated polynucleotide comprising a nucleic acid sequence encoding the polypeptides of claim 17.

54. A nucleic acid construct comprising the isolated polynucleotide of claim 53.

55. The isolated cyclic polypeptide of claim 2, wherein an amino acid at position 312 is glycine, an amino acid at position 313 is proline and an amino acid at position 314 is glycine.

56. The isolated cyclic polypeptide of claim 2, comprising a single internal disulfide bond.

57. The isolated cyclic polypeptide of claim 2, wherein an amino acid at position 315 is arginine lysine or glutamine.

58. The isolated cyclic polypeptide of claim 2, wherein an amino acid at position 305 is lysine or arginine, and an amino acid at position 307 is isoleucine, leucine or valine and an amino acid at position 309 is isoleucine, leucine, methionine or valine

59. The isolated cyclic polypeptide of any of claim 2, wherein an amino acid at positions 319 and 320 are threonine or alanine.

60. The isolated cyclic polypeptide of claim 2, further comprising amino acids of an antigen presenting polypeptide.

61. The isolated cyclic polypeptide of claim 2, comprising an amino acid sequence of a T-helper epitope.

62. The isolated cyclic polypeptide of claim 61, wherein said T-helper epitope comprises amino acids of HIV p24 gag.

63. The isolated cyclic polypeptide of claim 61, wherein said T-helper epitope comprises an amino acid sequence as set forth in SEQ ID NOs: 25 or 26.

64. The isolated cyclic polypeptide of claim 61, being no more than 50 amino acids.

65. A vaccine comprising the polypeptide of claim 2, as an active agent and an immunologically acceptable carrier.

66. An article of manufacture comprising the vaccine of claim 65 and a CD4 mimic compound.

67. The isolated cyclic polypeptide of claim 2, consisting of naturally occurring amino acids.

68. An isolated polynucleotide comprising a nucleic acid sequence encoding the polypeptides of claim 67.


SEQUENCE LISTING (42 sequences)

SEQ ID NO: 1 - protein, 40 aa, Artificial sequence
  C4-V3 linear synthetic peptide
KQIINMWQEVGKAMYARPNNNTRKSIHIGPGRAFYTTGEI

SEQ ID NO: 2 - protein, 40 aa, Artificial sequence
  C4-V3 T303C-I323C synthetic peptide
KQIINMWQEVGKAMYARPNNNCRKSIHIGPGRAFYTTGEC

SEQ ID NO: 3 - protein, 40 aa, Artificial sequence
  C4-V3 K305C-T320C synthetic peptide
KQIINMWQEVGKAMYARPNNNTRCSIHIGPGRAFYTCGEI

SEQ ID NO: 4 - protein, 22 aa, Artificial sequence
  Biotinilated V3 synthetic peptide
GSGTRKSIHIGPGRAFYTTGEI

SEQ ID NO: 5 - protein, 22 aa, Artificial sequence
  Biotinilated V3  (T303C-I323C) synthetic peptide
GSGCRKSIHIGPGRAFYTTGEC

SEQ ID NO: 6 - protein, 22 aa, Artificial sequence
  Biotinilated V3  (K305C-T320C) synthetic peptide
GSGTRCSIHIGPGRAFYTCGEI

SEQ ID NO: 7 - protein, 19 aa, Artificial sequence
  Biotinilated C4 synthetic peptide
GSGKQIINMWQEVGKAMYA

SEQ ID NO: 8 - protein, 19 aa, Artificial sequence
  V3 linear JR-FL peptide
TRKSIHIGPGRAFYTTGEI

SEQ ID NO: 9 - protein, 19 aa, Artificial sequence
  V3  (I307C,T319C) peptide
TRKSCHIGPGRAFYCTGEI

SEQ ID NO: 10 - protein, 19 aa, Artificial sequence
  V3 (305C,T320C) peptide
RRCSIHIGPGRAFYTCGER

SEQ ID NO: 11 - protein, 18 aa, Artificial sequence
  V3 (R304C,G321C) peptide
TCKSIHIGPGRAFYTTCE

SEQ ID NO: 12 - protein, 18 aa, Artificial sequence
  V3 (T303C,E322C) peptide
CRKSIHIGPGRAFYTTGC

SEQ ID NO: 13 - protein, 19 aa, Artificial sequence
  V3 (T303C,I323C)
CRKSIHIGPGRAFYTTGEC

SEQ ID NO: 14 - protein, 7 aa, Artificial sequence
  Synthetic peptide (a.a. 298-304 of gp120)
RPNNNTR

SEQ ID NO: 15 - protein, 16 aa, Artificial sequence
  Synthetic peptide (a.a. 421-436 of gp120)
KQIIMNWQEVGKAMYA

SEQ ID NO: 16 - protein, 350 aa, Artificial sequence
  gp120 expressed polypeptide (88-492 gp120 V1/V2, N301Q, T388A)
GHHHHHHENLYFQGNVTEHFNMWKNNMVEQMQEDIISLWDQSLKPCVKLTPLCVSGAGSCDTSVITQACPKISFEPIPIHYCAPAGFAILKCNDKTFNGKGPCKNVSTVQCTHGIRPVVSTQLLLNGSLAEEEVVIRSDNFTNNAKTIIVQLKESVEINCTRPNNNTRKSIHIGPGRAFYTTGEIIGDIRQAHCNISRAKWNDTLKQIVIKLREQFENKTIVFNHSSGGDPEIVMHSFNCGGEFFYCNSTQLFNSTWNNNTEGSNNTEGNTITLPCRIKQIINMWQEVGKAMYAPPIRGQIRCSSNITGLLLTRDGGINENGTEIFRPGGGDMRDNWRSELYKYKVVKIE

SEQ ID NO: 17 - protein, 36 aa, Human immunodeficiency virus
  misc_feature: Exemplary amino acid sequences of V3 from strain HXB2
CTRPNNNTRKRIRIQRGPGRAFVTIGKIGNMRQAHC

SEQ ID NO: 18 - protein, 35 aa, Human immunodeficiency virus
  misc_feature: Exemplary amino acid sequences of V3 from starin JR-FL
CTRPNNNTRKSIHIGPGRAFYTTGEIIGDIRQAHC

SEQ ID NO: 19 - protein, 19 aa, Human immunodeficiency virus
  misc_feature: gp120 motif from JR-FL strain
TRKSIHIGPGRAFYTTGEI

SEQ ID NO: 20 - protein, 21 aa, Human immunodeficiency virus
  misc_feature: gp120 motif from HXB2 strain
TRKRIRIQRGPGRAFVTIGKI

SEQ ID NO: 21 - protein, 5 aa, Artificial sequence
  298-302 from the V3 domain
RPNNN

SEQ ID NO: 22 - protein, 17 aa, Human immunodeficiency virus
  misc_feature: Gag T-helper epitope 1 (GTH1)
YKRWIILGLNKIVRMYS

SEQ ID NO: 23 - DNA, 120 bp, Artificial sequence
  C4-V3 domain (T303C-I323C) expression cassette optimized for expression in E-coli
aaacagattattaatatgtggcaggaagtaggtaaagcaatgtatgcccgtccgaacaacaactgccgtaaatctatccatatcggtccgggtcgtgctttctataccaccggtgaatgc

SEQ ID NO: 24 - protein, 16 aa, Artificial sequence
  T helper epitope from the C4 domain (421-436)
KQIINMWQEVGKAMYA

SEQ ID NO: 25 - protein, 16 aa, Artificial sequence
  T helper epitope from the C4 domain (421-436) modified at position 429
KQIINMWQVVGKAMYA

SEQ ID NO: 26 - protein, 17 aa, Mus musculus
  misc_feature: T helper epitope derived from murine HSP60 458-474
NEDQKIGIEIIKRALKI

SEQ ID NO: 27 - protein, 19 aa, Artificial sequence
  V3 domain (T303C-I323C) peptide
CRKSIHIGPGRAFYTTGEC

SEQ ID NO: 28 - DNA, 72 bp, Artificial sequence
  V3 domain (T303C-I323C) expression cassette optimized for expression in E-coli
cgtccgaacaacaactgccgtaaatctatccatatcggtccgggtcgtgctttctataccaccggtgaatgc

SEQ ID NO: 29 - protein, 4 aa, Artificial sequence
  Synthetic peptide (a.a. 322-325 of gp120)
EIIC

SEQ ID NO: 30 - protein, 40 aa, Artificial sequence
  C4-V3 K305C,G321C synthetic peptide
KQIINMWQEVGKAMYARPNNNTRCSIHIGPGRAFYTTCEI

SEQ ID NO: 31 - protein, 40 aa, Artificial sequence
  C4-V3 T303C,E322C synthetic peptide
KQIINMWQEVGKAMYARPNNNCRKSIHIGPGRAFYTTGCG

SEQ ID NO: 32 - protein, 42 aa, Artificial sequence
  C4-V3 N301C,G325C synthetic peptide
KQIINMWQEVGKAMYARPNCNTRKSIHIGPGRAFYTTGEIIC

SEQ ID NO: 33 - protein, 23 aa, Artificial sequence
  V3 T303C,E322C synthetic peptide
RPNNNCRKSIHIGPGRAFYTTGC

SEQ ID NO: 34 - protein, 23 aa, Artificial sequence
  V3  N301C,G325C synthetic peptide
CNTRKSIHIGPGRAFYTTGEIIC

SEQ ID NO: 35 - protein, 41 aa, Artificial sequence
  C4-V3  N301C, I324C synthetic peptide
KQIINMWQEVGKAMYARPNCNTRKSIHIGPGRAFYTTGEIC

SEQ ID NO: 36 - protein, 22 aa, Artificial sequence
  V3  N301C, I324C synthetic peptide
CNTRKSIHIGPGRAFYTTGEIC

SEQ ID NO: 37 - protein, 3 aa, Artificial sequence
  Synthetic peptide corresponding to AA 298-300 from the V3 domain of gp120
RPN

SEQ ID NO: 38 - protein, 16 aa, Artificial sequence
  AA 304-319 from the V3 domain of gp120
RKSIHIGPGRAFYTTG

SEQ ID NO: 39 - protein, 18 aa, Artificial sequence
  V3 T303C,E322C synthetic peptide
CRKSIHIGPGRAFYTTGC

SEQ ID NO: 40 - protein, 22 aa, Artificial sequence
  Biotinilated V3  (K305C-G321C) synthetic peptide
GSGTRCSIHIGPGRAFYTTCEI

SEQ ID NO: 41 - protein, 21 aa, Artificial sequence
  Biotinilated V3  (T303C-E322C) synthetic peptide
GSGCRKSIHIGPGRAFYTTGC

SEQ ID NO: 42 - protein, 26 aa, Artificial sequence
  Biotinilated V3  (N301C-G325C) synthetic peptide
GSGCNTRKSIHIGPGRAFYTTGEIIC